(19) United States
(12) Patent Application Publication
(10) Pub. No.: US 2023/0357189 A1
(43) Pub. Date: Nov. 9, 2023

(54) MODIFIED FLUOROQUINOLONES AND USES THEREOF

(71) Applicant: Technion Research & Development Foundation Limited, Haifa (IL)

(72) Inventors: Timor BAASOV, Haifa (IL); Valery BELAKHOV, Haifa (IL); Moshe Nissim GOLDMEIER, Haifa (IL)

(73) Assignee: Technion Research & Development Foundation Limited, Haifa (IL)

(21) Appl. No.: 18/221,513

(22) Filed: Jul. 13, 2023

Related U.S. Application Data
(63) Continuation of application No. PCT/IL2022/050059, filed on Jan. 14, 2022.
(60) Provisional application No. 63/137,300, filed on Jan. 14, 2021.

(51) Int. Cl. C07D 401/12; C07F 15/06; C07F 3/06; A61P 31/04

(52) U.S. Cl. CPC C07D 401/12; C07F 15/06; C07F 3/06; A61P 31/04

(57) ABSTRACT

Modified quinolones (e.g., fluoroquinolones) featuring a quinolone (e.g., fluoroquinolone) skeleton having conjugated thereto, via selected linkers, a metal chelating moiety, and metal complexes thereof, are provided. Uses of the compounds and complexes in treating medical conditions associated with pathogenic microorganism are also provided.

### RELATED APPLICATIONS

This application is a Continuation of PCT Patent Application No. PCT/IL2022/050059 having International filing date of Jan. 14, 2022, which claims the benefit of priority under 35 USC § 119(e) of U.S. Provisional Patent Application No. 63/137,300 filed on Jan. 14, 2021. The contents of the above applications are all incorporated by reference as if fully set forth herein in their entirety.

### FIELD AND BACKGROUND OF THE INVENTION

The present invention, in some embodiments thereof, relates to antibacterial agents and, more particularly, but not exclusively, to newly designed antibacterial agents featuring a modified fluoroquinolone structure, and to uses thereof in treating medical conditions associated with a pathogenic microorganism, optionally via a catalytic mechanism.

Fluoroquinolones are highly potent, broad spectrum antibiotics that are among the most commonly prescribed antibacterials (antibiotics, antibacterial agents) in the world. Examples include ciprofloxacin (Cipro), as well as moxifloxacin and geranoaxacin. The fluoroquinolone antibiotics exert a bacteriostatic effect by selectively binding to the bacterial topoisomerase IIA-DNA complex and thereby inhibiting DNA replication. At higher doses (5-10 MIC), they exert a bactericidal effect by causing fragmentation of the bacterial chromosome, which is toxic for the bacteria.

However, the emergence of resistance is becoming a critical issue that is limiting the use of this class of antibiotics. Consequently, several novel bacterial topoisomerase type IIA inhibitors have been developed that retain potency against quinolone-resistance bacterial strains by offering an alternative binding mode and/or mechanism of action, including, among others, NXL101 and REDX07638 [Charrier et al. Antimicrob. Agents Chemother. 2017, 61 (5)].

It has been well documented that once a new antibiotic is introduced into the clinic, whether it is a novel chemical entity acting at a distinct bacterial target or a semisynthetic derivative that counters the resistance to its parent drug, it is only a short matter of time until new resistance will yet again emerge and create a public health problem. Consequently, it would seem apparent that changing the mechanism of inhibition and/or binding mode at the target does not necessarily address the fundamental problem of delaying resistance development; it simply ‘buys more time’ for the topoisomerase-targeting antibacterial fluoroquinolones. The significance of this health problem has prompt searching for novel approaches that would allow humans to stay more than one step ahead.

One such approach is the development of catalytic antibiotics as small molecule-based therapeutic agents to mediate catalytic inactivation of a specific bacterial target to form an inactive or dysfunctional entity. Inhibition occurs in at least two steps and in a manner analogous to the Michaelis-Menten enzyme model: The compound must first bind non-covalently to the target; the resulting complex then undergoes specific chemical modification(s), which results in the deleterious transformation of the target and the release of the drug for another cycle, as shown in Background Art FIG. 1. Thus, unlike conventional antibiotics that inhibit their targets by either non-covalent or covalent interactions, catalytic antibiotics should promote multiple turnovers of a catalytic cycle.

There are many advantages to this approach, including improved potency and lower toxicity (due to lower dosage requirements), and general applicability to targets with weak binding sites. Further, this approach is likely to delay resistance for the following reasons: Firstly, individual target-based resistance mutations are unlikely to affect the k₂ rate constant even if they do affect the rate at which the inhibited complex forms (Kᵢ), such that catalytic antibiotics would be able to target mutants that have acquired energetically ‘shallow’ binding sites to the pharmacophore scaffold, since the efficiency of such ‘enzymatic’ systems is governed by the ratio of these two parameters and not just by Kᵢ. Thus, given enough exposure to the compound, even mutants that react considerably more slowly will become deactivated and so resistance is likely to develop more slowly. Secondly, the killing activity of catalytic antibiotics are expected to be totally independent of cellular processes such as protein synthesis and/or anaerobic conditions. Such a feature could be particularly important with pathogens such as Mycobacterium tuberculosis, that enter a dormant state in which they become tolerant to many antimicrobial agents.

In the past few decades, artificial enzymes that target nucleic acids or proteins have been well studied. However, in most cases the reported catalysts suffer from a lack of substrate selectivity, which can lead to lower catalyst efficiency, side effects and toxicity. One approach for addressing this issue is to join a substrate selective binding motif to the catalytic warhead, as also shown in Background Art FIG. 1. Several studies have recently reported on multifunctional, antibacterial metallopeptides that modify nucleic acids, proteins or phospholipids via ROS generated by an Amino Terminal Copper and Nickel (ATCUN) binding motif. For example, conjugates of Cu(II)-ATCUN with known antimicrobial peptides demonstrated significant improvement in antibacterial activity relative to parent peptides [Joyner et al. Chem Commun 2013, 49 (21), 2118-2120; Alexander et al. ACS Chem. Biol. 2019, 14 (3), 449-458]. These and other reports [Libardo et al. FEBS J. 2017, 284 (21), 3662-3683; Libardo et al. ACS Infect. Dis. 2018, 4 (11), 1623-1634; Wang et al. ACS Chem. Biol. 2017, 12 (5), 1170-1182] show that the attachment of a metal-binding motif can promote oxidative damage and enhance the antibacterial activity of known antibacterial peptides. However, the conservative changes in MIC observed versus the parent peptides were not as high as would be expected from a catalytic metallodrug, which suggests that more advanced design strategies are needed to realize the full clinical potential of catalytic antibiotics.

Additional background art includes Goldmeier et al., CS Infect. Dis. 2021, 7, 3, 608-623;
program(dot)eventact(dot)com/Agenda/Lecture/193863?code=4110641; and
program(dot)eventact(dot)com/Agenda/Lecture/210820?code=4160498.

### SUMMARY OF THE INVENTION

Embodiments of the present invention relate to modified fluoroquinolone compounds (e.g., modified fluoroquinolone-based antibiotics), to metal complexes thereof (also referred to herein as fluoroquinolone-nuclease conjugates), and to uses thereof in the treatment of medical conditions associated with a pathogenic microorganism (e.g., a bacterium), optionally via a catalytic mechanism.

According to an aspect of some embodiments of the present invention there is provided a compound represented by Formula IIa, IIb or IIc:

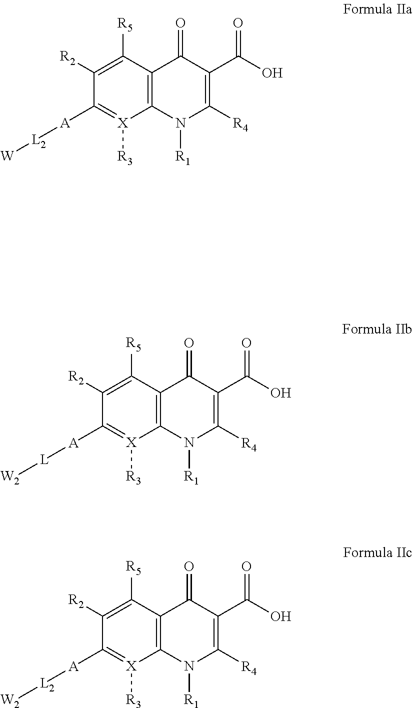

or a pharmaceutically acceptable salt thereof,
wherein:
X is C or N, wherein when X is C, the dashed line represents a bond, and when X is N, R₃ is absent;
R₁ is hydrogen, alkyl, aryl, heteroaryl or cycloalkyl, or alternatively, R₁ and R₄ or R₁ and R₃ form together a heterocyclic ring;
R₂ is hydrogen or halo;
R₃, if present, is hydrogen, alkyl, halo, alkoxy, thioalkoxy, aryloxy, thioaryloxy, aryl, heteroaryl, cyano (nitrile) or, alternatively, forms with R₁ a heterocyclic (heteroaryl or heteroalicyclic) ring;
R₄ is hydrogen, alkyl, cycloalkyl, or halo, or, alternatively, forms with R₁ the heterocyclic ring;
R₅ is hydrogen, alkyl or cycloalkyl;
A is or comprises a heterocyclic moiety, or is or comprises a cycloalkyl substituted by an amine;
L is a linking moiety (a linker) being from 6 to 10 carbon atoms in length, which can be aliphatic (non-aromatic) or aromatic;
W is a heteroalicyclic or a heteroaliphatic metal chelating moiety;
L₂ is a linking moiety (a linker) being from 5 to 10 carbon atoms in length, which can be aliphatic (non-aromatic) or aromatic, and which has a moiety P that comprises a heteroatom-containing group attached thereto, wherein the heteroatom-containing group is an amine; and
W₂ is a heteroalicyclic or a heteroaliphatic metal chelating moiety which has a moiety P that comprises a heteroatom-containing group that is non-protonated or not fully protonated at physiological pH and/or is capable of reversibly binding to a metal ion when associated with the W attached thereof, wherein the heteroatom-containing group is an amine.

According to some of any of the embodiments described herein, R₁ is a cycloalkyl (e.g., cyclopropyl); and/or R₂ is halo (e.g., fluoro); and/or R₄ and R₅ are each hydrogen.

According to some of any of the embodiments described herein, A is a heteroalicyclic.

According to some of any of the embodiments described herein, A is an amine-containing heteroalicyclic (e.g., piperazine).

According to some of any of the embodiments described herein, L₂ is an aliphatic (non-aromatic) linker.

According to some of any of the embodiments described herein, L₂ is a non-aromatic hydrocarbon chain of 5 to 10, or of 5 to 9, or of 6 to 8, carbon atoms in length, wherein at least one carbon of the hydrocarbon chain is substituted by the moiety P that comprises the heteroatom-containing group.

According to some of any of the embodiments described herein, the carbon atom in the hydrocarbon chain that is substituted by the moiety P is separated from the W by 0, 1 or 2 carbon atoms, preferably 0 or 1 carbon atoms.

According to some of any of the embodiments described herein, L₂ is 6 or 7 carbon atoms in length.

According to some of any of the embodiments described herein, L₂ is an aromatic linker that comprises at least one aryl in its chain or as a substituent.

According to some of any of the embodiments described herein, L₂ is a hydrocarbon chain that comprises at least one aryl in its chain or at least one aryl substituent.

According to some of any of the embodiments described herein, L₂ is —(CRaRb)-Aryl-(CRcRd)-, or —(CRaRb)-Aryl-(CRcRd)-(CReRf)—, wherein Ra-Rd, and Re and Rf, if present, are each independently hydrogen, alkyl or the moiety P that comprises the heteroatom-containing group, and wherein the Aryl is substituted by the moiety P.

According to some of any of the embodiments described herein, each of Ra-Rd, and Re or Rf, if present, is hydrogen.

According to some of any of the embodiments described herein, L₂ is —(CRaRb)-Aryl-(CRcRd)-, or —(CRaRb)-Aryl-(CRcRd)-(CReRf)—, wherein Ra-Rd, and Re and Rf, if present, are each independently hydrogen, alkyl or the moiety P that comprises the heteroatom-containing group, at least one of the Ra-Rd, and Re and Rf, if present, is the moiety P.

According to some of any of the embodiments described herein, the Aryl is unsubstituted or is substituted by one or more of halo, alkyl, cycloalkyl and the moiety P that comprises the heteroatom-containing group.

According to some of any of the embodiments described herein, W₂ is a heteroalicyclic metal chelating moiety that is substituted by the moiety P that comprises the heteroatom-containing group.

According to some of any of the embodiments described herein, the moiety P is attached to a heteroatom in the heteroalicyclic moiety which is ortho to the attachment point of W₂ to the L or L₂.

According to some of any of the embodiments described herein, the heteroatom-containing group is a primary amine.

According to some of any of the embodiments described herein, the moiety P that comprises the heteroatom-containing group comprises a hydrocarbon of from 1 to 8 carbon atoms in length, which is terminated or substituted by the heteroatom-containing group.

According to an aspect of some embodiments of the present invention there is provided a compound represented by Formula I:

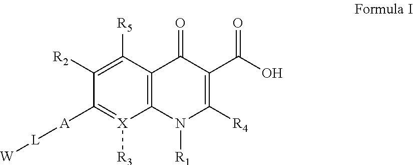

or a pharmaceutically acceptable salt thereof,
wherein:
X is C or N, wherein when X is C, the dashed line represents a bond, and when X is N, R₃ is absent, as described herein in any of the respective embodiments;
R₁ is hydrogen, alkyl, aryl, heteroaryl or cycloalkyl, or alternatively, R₁ and R₄ or R₁ and R₃ form together a heterocyclic ring, as described herein in any of the respective embodiments;
R₂ is hydrogen or halo;
R₃, if present, is hydrogen, alkyl, halo, alkoxy, thioalkoxy, aryloxy, thioaryloxy, aryl, heteroaryl, cyano (nitrile) or, alternatively, forms with R₁ a heterocyclic (heteroaryl or heteroalicyclic) ring, as described herein in any of the respective embodiments;
R₄ is hydrogen, alkyl, cycloalkyl, or halo, or, alternatively, forms with R₁ the heterocyclic ring, as described herein in any of the respective embodiments;
R₅ is hydrogen, alkyl or cycloalkyl, as described herein in any of the respective embodiments;
A is or comprises a heterocyclic moiety, or is or comprises a cycloalkyl substituted by an amine, as described herein in any of the respective embodiments;
L is a linking moiety (a linker) being from 6 to 10 carbon atoms in length, which can be aliphatic or aromatic, as described herein in any of the respective embodiments; and
W is a heteroalicyclic or a heteroaliphatic metal chelating moiety, as described herein in any of the respective embodiments.

According to some of any of the embodiments described herein, L is an alkylene chain of 6 or 7 carbon atoms in length, preferably of 7 carbon atoms in length.

According to some of any of the embodiments described herein, at least one carbon of the alkylene chain is substituted by a moiety P that comprises a heteroatom-containing group that is non-protonated or not fully protonated at physiological pH and/or is capable of reversibly binding to the metal ion.

According to some of any of the embodiments described herein, the carbon atom in the alkylene chain that is substituted by the moiety P that comprises the heteroatom-containing moiety is separated from the W by 0, 1 or 2 carbon atoms, preferably 0 or 1 carbon atoms.

According to some of any of the embodiments described herein, L is an aromatic linker which comprises at least one aryl, preferably phenyl.

According to some of any of the embodiments described herein, L is —(CRaRb)-Aryl-(CRcRd)-, or —(CRaRb)-Aryl-(CRcRd)-(CReRf)—, wherein Ra-Rd, and Re and Rf, if present, are each independently hydrogen, alkyl or a moiety P that comprises a heteroatom-containing group that is non-protonated or not fully protonated at physiological pH and/or is capable of reversibly binding to the metal ion (e.g., in physiological environment).

According to some of any of the embodiments described herein, L is (CRaRb)-Aryl-(CRcRd).

According to some of any of the embodiments described herein, each of Ra-Rd, and of Re and Rf, if present, is hydrogen.

According to some of any of the embodiments described herein, the aryl is unsubstituted or is substituted by one or more of halo, alkyl, cycloalkyl and a moiety P that comprises a heteroatom-containing group that is non-protonated or not fully protonated at physiological pH and/or is capable of reversibly binding to the metal ion (e.g., in physiological environment).

According to some of any of the embodiments described herein, aryl is substituted by the moiety P that comprises the heteroatom-containing group.

According to some of any of the embodiments described herein, at least one of Rc-Rd, and Re and Rf, if present, is the moiety P that comprises the heteroatom-containing moiety.

According to some of any of the embodiments described herein, the W metal chelating moiety is substituted by a moiety P that comprises a heteroatom-containing group that is non-protonated or not fully protonated at physiological pH and/or is capable of reversibly binding to the metal ion (e.g., in physiological environment).

According to some of any of the embodiments described herein, the W is a heteroalicyclic moiety, and wherein the moiety P that comprises the heteroatom-containing group is attached to a heteroatom in the heteroalicyclic chelating moiety which is ortho to the attachment point to the L.

According to some of any of the embodiments described herein, the compound comprises at least one moiety P that comprises a heteroatom-containing group that is non-protonated or not fully protonated at physiological pH and/or is capable of reversibly binding to the metal ion (e.g., in physiological environment).

According to some of any of the embodiments described herein, the compound represented by Formula Ia or Formula Ib:

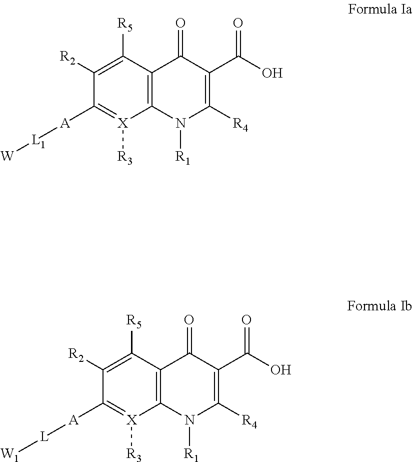

or a pharmaceutically acceptable salt thereof,
wherein:
R₁, R₂, R₃, R₄, R₅, A, L and W are as defined for Formula I;
L₁ is a non-aromatic hydrocarbon chain of from 5 to 10, or from 5 to 9, carbon atoms in length, wherein at least one carbon of the hydrocarbon chain is substituted by the moiety P that comprises the heteroatom-containing group; or
L₁ is an aromatic linker that comprises at least one aryl, wherein the aryl is substituted by the moiety P that comprises the heteroatom-containing group; and
W₁ is a heteroalicyclic metal chelating moiety that is substituted by the moiety P that comprises the heteroatom-containing group.

According to some of any of the embodiments described herein, the heteroatom-containing group comprises at least one amine group which is non-protonated or not fully protonated at physiological pH.

According to some of any of the embodiments described herein, the heteroatom-containing group is a guanidine group.

According to some of any of the embodiments described herein, the heteroatom-containing group is a primary amine group.

According to some of any of the embodiments described herein, the moiety P that comprises the heteroatom-containing group comprises a hydrocarbon of from 1 to 8 carbon atoms in length, which is terminated or substituted by the heteroatom-containing group.

According to some of any of the embodiments described herein, (e.g., of any of Formulae I, Ia, Ib, IIa, IIb, IIc, IV, IVa, IVb, Va, Vb and Vc), W is a metal chelating moiety that, when having a metal ion chelated therewith, is capable of acting as DNA nuclease (capable of cleaving DNA).

According to some of any of the embodiments described herein, W is a heteroalicyclic metal chelating moiety, as described herein in any of the respective embodiments, and in some embodiments W is cyclen.

According to an aspect of some embodiments of the present invention there is provided a complex comprising the compound of any one of claims 1-46 and a metal ion associated with the W.

According to some of any of the embodiments described herein, the metal is a redox reactive and/or Lewis acid metal.

According to some of any of the embodiments described herein, the metal is selected from copper, cobalt, zinc, magnesium, iron, nickel and manganese.

According to some of any of the embodiments described herein, the metal ion is Cu(II).

According to some of any of the embodiments described herein, the metal ion is Co(III).

According to some of any of the embodiments described herein, the metal ion is Zn(II).

According to an aspect of some embodiments of the present invention there is provided a metal complex comprising the compound of Formula IIa, IIb or IIc and a metal ion associated with the W metal chelating moiety, wherein the metal ion is Co(III).

According to an aspect of some embodiments of the present invention there is provided a metal complex comprising the compound of Formula Ia or Ib and a metal ion associated with the W metal chelating moiety, wherein the metal ion is Co(III).

According to some of any of the embodiments described herein, a compound or a complex as described herein is capable of cleaving a bacterial DNA and/or of interfering with bacterial DNA gyrase activity.

According to some of any of the embodiments described herein, a compound or a complex as described herein is capable of fragmenting bacterial supercoiled plasmid DNA into linear DNA.

According to some of any of the embodiments described herein, a compound or a complex as described herein is capable of reducing a population of a pathogenic microorganism (e.g., a bacterium) in or on a substrate.

According to some of any of the embodiments described herein, reducing the population is catalytic.

According to an aspect of some embodiments of the present invention there is provided a pharmaceutical composition comprising a compound or a complex as described herein in any of the respective embodiments and any combination thereof, and optionally a pharmaceutically acceptable carrier.

According to an aspect of some embodiments of the present invention there is provided a compound or a complex as described herein in any of the respective embodiments and any combination thereof, for use in the treatment a medical condition associated with a pathogenic microorganism.

According to some of any of the embodiments described herein, the pathogenic microorganism is a bacterium.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 (Background Art) presents a general scheme representing the mechanism for catalytic deactivation of a biomolecular target, according to Yu, Z. and Cowan, J. A., Chem.—A Eur. J. 2017, 23 (57), 14113-14127; Singh et al. Nat. Rev. Drug Discov. 2011, 10 (4), 307-317; and Suh, J., Asian J. Org. Chem. 2014, 3 (1), 18-32.

FIG. 2 is a 3D representation of the ternary complex as obtained from an X-ray crystal structure (PDB ID code 2XKK [Wohlkonig et al. Nat. Struct. Mol. Biol. 2010, 17 (9), 1152-1153]). The ParE28-ParC58 fusion truncate of Acinetobacter Baumannii Topoisomerase IV (Topo IV) shown as an electrostatic potential map (blue=+ve, red=−ve) in complex with the fluoroquinolone moxifloxacin (shown as spheres; carbon=green, nitrogen=blue) and DNA (shown as orange/black cartoon); image generated using PyMOL Molecular Graphics System, Version 2.0.4 Schrödinger, LLC. 2017. The yellow arrows mark out the scissile phosphodiester bonds adjacent to the fluoroquinolone molecules.

FIG. 3 is a scheme showing hydrolytic (blue arrows) and oxidative (purple arrows) DNA cleavage pathways.

FIGS. 4A-B present docking poses of energy minimum of low energy cluster for exemplary complexes according to the present embodiments, 5-Co(III) (FIG. 4A) and 5-Cu(II) (FIG. 4B) in crystal structure 2XKK [Wohlkonig et al. Nat. Struct. Mol. Biol. 2010, 17 (9), 1152-1153]. For clarity, only the metal-cyclen warhead and the adjacent DNA residues are shown (green carbons=cyclen, white carbons=DNA, metal=magenta). Blue dashed lines indicate distance between metal-activated water and scissile phosphodiester bond. Yellow dashed line indicates electrostatic interaction between phosphate oxygen and N—H^{δ+}.

FIGS. 5A-B present the structures of exemplary ciprofloxacin derivatives according to some embodiments of the present invention, designated Compounds 1-6 (FIG. 5A) and an exemplary synthetic scheme for preparing these compounds (FIG. 5B). Reagents and conditions: (a) Dowex 50WX8, MeOH, reflux, 81%; (b) DIPEA, CH₃CN, 60° C., Br(CH₂)₆Br, 54% (8a), Br(CH₂)₇Br, 61% (8b), Br(CH₂)₈Br, 58% (8c), 1,4-Bis(bromomethyl)-benzene, 22% (8d); (c) DIPEA, CH₃CN, 0° C. to room temperature, 1-(2-bromoethyl)-4-(bromomethyl)benzene, 60% (8e), 1-(bromomethyl)-4-(3-bromopropyl)-benzene, 63% (8f); (d) cyclen, Cs₂CO₃, CH₃CN, 60° C.; (e) Boc₂O, Et₃N, DCM, 0° C. to room temperature, 45% over 2 steps (9a), 33% over 2 steps (9b), 49% over 2 steps (9c), 34% over 2 steps (9e), 55% over 2 steps (9f); (f) tri-boc-cyclen, DIPEA, DCM, room temperature, 72% (9d); (g) LiOH, MeOH:H₂O 5:1, room temperature; (h) TFA, DCM, room temperature, AmberLite™, 48% over 2 steps (1), 49% over 2 steps (2), 61% over 2 steps (3), 51% over 2 steps (4), 38% over 2 steps (5), 59% over 2 steps (6).

FIG. 5C presents an exemplary synthetic scheme for preparing dibromo compounds 10 (Part A) and 11 (Part B) used in the syntheses shown in FIG. 5B. Reagents and condition (a) BH₃·SMe₂, THF, 0° C., 94%; (b) HBr, acetic acid (AcOH), 100° C., 59% (10), 80% (11); (c) propargyl alcohol, PdCl₂(PPh3)₂, CuI, Et₃N, DMF, room temperature, 84%; (d) tert-butyldimethylsilyl chloride (TBSCl), imidazole, DMF, room temperature, 94%; (e) H₂(g) 1 atm, Pd/C 10%, ethyl acetate (EtOA), 88%.

FIG. 6 presents the data obtained in comparative cleavage experiments of (+) supercoiled pHOT-1 plasmid (0.007 μg·L⁻¹) in HEPES buffer (50 mM, pH 7.4) over 5 hours, in the presence of the exemplary compounds 1, 2, 4 and 5, with and without chelated Cu(II) ions.

FIGS. 7A-C are bar graphs (upper panels) and agarose gel images (lower panels) presenting the data obtained in comparative concentration dependent cleavage of (+) supercoiled pHOT-1 plasmid (0.007 μg·L⁻¹) in HEPES buffer (50 mM, pH 7.4) over 5 hours. Standard deviations of three independent experiments are shown as error bars for Cu(II) alone (CuCl₂) and its complexes with cyclen, Compounds 1 and 4 (FIG. 7A), Compounds 2 and 5 (FIG. 7B), and Compounds 3 and 6 (FIG. 7C). The agarose gel images show one representative experiment.

FIG. 7D presents a bar graph (upper panel) and an agarose gel image (lower panel) showing the cleavage of (+) supercoiled pHOT-1 plasmid (0.007 μg·L⁻¹) in HEPES buffer (50 mM, pH 7.4) with Complex 5-Cu(II) (0.5 mM) alone or with a series of scavenging compounds (10 mM), over 5 hours.

FIG. 8 presents the data obtained in a cleavage experiment of (+) supercoiled pHOT-1 plasmid (0.007 g·L⁻¹) in HEPES buffer (50 mM, pH 7.4) over 0.5 hours in the presence of Co(III)-cyclen complex and complexes of Co(III) with Compounds 1-6.

FIGS. 9A-C are bar graphs (upper panels) and agarose gel images (lower panels) presenting the data obtained in concentration dependent cleavage of (+) supercoiled pHOT-1 plasmid (0.007 g·L⁻¹) in HEPES buffer (50 mM, pH 7.4) and ascorbic acid (0.32 mM) over 2 hours, in the presence of Cu(II)-cyclen and Cu(II) complexes with Compounds 1 and 4 (FIG. 9A), Compounds 2 and 5 (FIG. 9B), and Compounds 3 and 6 (FIG. 9C). ‘-Asc’=No ascorbate. Standard deviations of three independent experiments are shown as error bars. The agarose gel images show one representative experiment.

FIG. 9D is a bar graph (upper panel) and an agarose gel image (lower panel) presenting the data obtained in concentration dependent cleavage of (+) supercoiled pHOT-1 plasmid (0.007 g·L⁻¹) in HEPES buffer (50 mM, pH 7.4) in the presence of 5-Cu(II) (0.01 mM), ascorbic acid (0.32 mM) and a series of radical scavengers (10 mM each) over 2 hours.

FIG. 10A is a dose-response curve (upper panel) and an agarose gel image (lower panel) presenting the data obtained upon incubation of E. coli DNA gyrase with relaxed pHOT-1 plasmid (0.007 μg·L⁻¹), in Tris-HCl buffer (35 mM, pH 7.5) together with KCl (24 mM), MgCl₂ (4 mM), DTT (2 mM), Spermidine (1.8 mM), 6.5% (w/v) glycerol, bovine serum albumin (0.1 mg/ml), ATP (1 mM) and various concentrations of Compound 6. The exemplary gel image shows one example of the three independent experiments.

FIG. 10B are gel images presenting the data obtained in the DNA gyrase assay of FIG. 10A, in the presence of various concentrations of ciprofloxacin, and Compounds 1, 2, and 4. For each of the compounds, ciprofloxacin, 1 and 4, the experiment was performed twice, once with and once without a second incubation with SDS and Proteinase K, while for 2 it was only performed in the presence of Proteinase K.

FIGS. 11A-B are comparative plots (FIG. 11A) and respective gel images (FIG. 11B) presenting the data obtained in the presence of Cipro, 1-Cu(II), 2-Cu(II) and 4-Cu(II) incubated with E. coli DNA gyrase and (+) supercoiled (SC) DNA (0.009 μg·μL⁻¹), using the same conditions as the supercoiling assay except for the absence of ATP, followed by additional incubation with Proteinase K (left panels) and without Proteinase K treatment (right panels).

FIGS. 12A-B show the stabilization of the ternary complex by fluoroquinolone moxifloxacin (PDB ID code 2XKK) (FIG. 12A) and its destabilization by catalytic cleavage of the DNA backbone by 5-Cu(II) (FIG. 12B); prepared from docking results for 5-Cu(II) in 2XKK (DNA=orange, Mg²⁺=green, moxi=yellow, DNA gyrase tyrosine=red structural formula, 5-Cu(II)=magenta).

FIGS. 13A-B are gel images presenting the data obtained for cleavage of (+) supercoiled pHOT-1 plasmid (0.007 μg·L⁻¹) in the presence of 5-Cu(II) (0.5 mM) over 2.5 hours in HEPES buffer (50 mM, pH 7.4) or in Tris-HCl buffer (35 mM, pH 7.5), alone (lane 5) or together with KCl (24 mM, lane 6), MgCl₂ (4 mM, lane 7)), DTT (2 mM, lane 8), 6.5% (w/v) glycerol (lane 9), bovine serum albumin (0.1 mg/mL, lane 10) or together with all these ingredients (lane 11) (FIG. 13A), and in the presence of increasing concentrations of spermidine (FIG. 13B).

FIG. 14 are gel images showing a concentration dependent cleavage of (+) supercoiled pHOT-1 plasmid (0.007 μg·L⁻¹) in HEPES buffer (50 mM, pH 7.4) and ascorbic acid (0.32 mM) or in TopoIV buffer (i.e. 40 mM HEPES (pH 7.4), 100 mM potassium glutamate, 10 mM Magnesium Acetate, 250 μg BSA/mL and 1.8 mM ATP), in the presence of increasing concentrations of 4-Cu(II) over 2 hours.

FIGS. 15A-B are gel images presenting the data obtained upon incubation of 5-Cu(II) with (+) supercoiled pHOT-1 plasmid (0.007 g·L⁻¹) in HEPES buffer (36 mM, pH 7.4) without (FIG. 15A; t=5 hours) or with (FIG. 15B; t=2 hours) ascorbic acid (0.32 mM) and with 100 mM potassium glutamate, 10 mM Magnesium Acetate, 250 μg BSA/ml or 1.8 mM ATP (i.e. the ingredients of TopoIV buffer).

FIGS. 16A-B present UV-VIS spectra of Cu(II)-cyclen (3.3 mM) before and after the addition of potassium glutamate (660 mM) at room temperature (FIG. 16A), like the ratio between 5-Cu(II) and potassium glutamate in the hydrolytic inhibition assay (i.e. 1:200; see, FIG. 15A), showing a small shift in the λₘₐₓ but a significant shift from the reported visible-region λₘₐₓ (571 nm) of the major species (above neutral pH) of the copper glutamate complex ([Cu(Glu)₂(H₂O)₂]·2H₂O); and gel images showing a concentration dependent inhibition of cleavage of (+) supercoiled pHOT-1 plasmid (0.007 g·L⁻¹) in HEPES buffer (50 mM, pH 7.4) in the presence of 5-Cu(II) (0.5 mM) over 5 hours, in the presence of potassium glutamate or KCl (FIG. 16B).

FIG. 17 presents UV-VIS spectra of Cu(II)-cyclen (3.3 mM) before and after the addition of ATP (11.9 mM) at room temperature, like the ratio between 5-Cu and ATP in the hydrolytic inhibition assay (i.e. 1:3.6; see, FIG. 15A). As depicted, there is a significant shift in the λₘₐₓ and the value is significantly shifted from the reported λₘₐₓ (˜800 nm) of the major species (at pH=5.5-8.0) of the complex between ATP and Cu(II).

FIG. 18 presents the chemical structures of additional exemplary compounds according to some embodiments of the present invention, which feature a cyclen “warhead” and an exemplary guanidine-containing pendant moiety.

FIG. 19 presents a docking pose of an exemplary compound featuring a guanidine-containing pendant moiety modelled onto the cyclen ring at the ortho position docked into the crystal structure 2XKK as described herein.

FIGS. 20A-B present synthetic schemes of exemplary compounds featuring an aliphatic (FIG. 20A) or aromatic (FIG. 20B) linker and an exemplary guanidine-containing pendant moiety attached to the cyclen moiety according to some embodiments of the present invention.

FIGS. 21A-B present synthetic schemes of exemplary compounds featuring an aliphatic (FIG. 21A) or aromatic (FIG. 21B) linker and an exemplary guanidine-containing pendant moiety attached to the linker according to some embodiments of the present invention. Ref*=J. Musacchio, B. C. Lainhart, X. Zhang, S. G. Naguib, T. C. Sherwood, R. R. Knowles, Science (80-.). 2017, 355, 727-730.

FIG. 22 presents the chemical structures of exemplary fluoroquinolone antibiotics, as taken from “Antibiotics: Challenges, Mechanisms, Opportunities. Editors: Christopher Walsh, Timothy Wencewicz. ASM Press, 2016 (print ISBN 9781555819309, e-ISBN 9781555819316)”.

FIGS. 23A-B present the chemical structures of Compounds 19-22, additional exemplary compounds according to some embodiments of the present invention, which feature a cyclen “warhead” and an exemplary guanidine-containing pendant moiety (FIG. 23A) and an exemplary synthetic scheme of these exemplary compounds (FIG. 23B).

FIGS. 24A-C present the chemical structures of additional exemplary compounds according to some embodiments of the present invention, which feature a cyclen “warhead” and an exemplary primary amine-containing pendant moiety (FIG. 24A), the chemical structures of exemplary such compounds, denoted as Compounds 23-26, (FIG. 24B) and an exemplary synthetic scheme of these exemplary compounds (FIG. 24C).

FIG. 25 presents comparative EPR spectra of two exemplary Cu-ligand complexes according to some embodiments of the present invention, 21-Cu(II) and 5-Cu(II), at varying pH.

FIGS. 26A-B present comparative ¹³C NMR spectra (FIG. 26A) and the synthetic scheme (FIG. 26B) of Compound 21 and activated complexes thereof, 21-Co(III): Co(III)-21(H₂O)(⁻OH) and Co(III)-21(H₂O).

FIGS. 27A-C present gel images showing cleavage of (+) supercoiled pHOT-1 plasmid (0.4 μg) in the presence of 2-Cu(II), and Compounds 19-22 and complexes thereof with Cu(II), Zn(II) and Co(III) (FIG. 27A), and in the presence of Compounds 23 and 26 with Cu(II), Zn(II) and Co(III) (FIG. 27B), in HEPES buffer (50 mM, pH 7.4) over 5 hours, upon post treatment with EDTA (50 mM) and resin (5 mg). FIG. 27C present gel images comparing the data obtained in the presence of 2-Cu(II) and Compounds 19, 22 and 23 in the same experiment with 0.6 μg plasmid.

FIG. 28 is a scheme presenting a suggested equilibrium of Cu(II) complexes of Compounds 19-22 at physiological pH.

FIGS. 29A-B are a bar graph (FIG. 29A) and gel images (FIG. 29B) presenting the data obtained in concentration dependent cleavage of (+) supercoiled pHOT-1 plasmid (0.6 μg) with 2-Cu(II), 23-Co(III), and 26-Co(III) in HEPES buffer (50 mM, pH 7.4) over 5 hours, upon post treatment with EDTA (50 mM) and resin (5 mg).

FIG. 30 is a scheme presenting a suggested equilibrium of capped and uncapped complexes of 23-Co(III).

FIGS. 31A-B present gel images showing dose-dependent DNA cleavage assays of the complexes 2-Cu(II), 23-Co(III) (FIG. 31A) and Co(III)-cyclen (FIG. 31B) in 50 mM TRIS buffer, without additives and in the presence of 100 mM potassium glutamate and 1.8 mM ATP.

FIGS. 32A-B present gel images showing DNA cleavage assays of Co(III)-cyclen and 4-Co(III) in the presence of 0.2 μg DNA over 2 hours (FIG. 32A), and of 2-Cu(II) or 23-Co(III) in the presence of 0.6 μg DNA over 10 hours (FIG. 32B). Schemes of complexes 4-Co(III) and 23-Co(III) at physiological pH are depicted below each FIGS. 32A and 32B, respectively.

FIGS. 33A-B present chemical structures of exemplary compounds according to some embodiments of the present invention.

### DESCRIPTION OF SPECIFIC EMBODIMENTS OF THE INVENTION

The present invention, in some embodiments thereof, relates to antibacterial agents and, more particularly, but not exclusively, to newly designed antibacterial agents featuring a modified fluoroquinolone structure, and to uses thereof in treating medical conditions associated with a pathogenic microorganism, optionally via a catalytic mechanism.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have set out to explore catalytic antibiotics that are designed to cleave a specific, ‘critical’ chemical bond in a bacterial target that is projected to result in the immediate deactivation of the target.

The present inventors have focused on re-designing ciprofloxacin and other fluoroquinolone antibiotics, to catalytically cleave a specific, scissile phosphodiester bond at the site of the fluoroquinolone-topoisomerase-DNA ternary complex, where the bound DNA is (a) stretched [Bax et al. J. Mol. Biol. 2019, 431 (18), 3427-3449] and (b) free of significant binding interactions with the surrounding residues, as shown in FIG. 2.

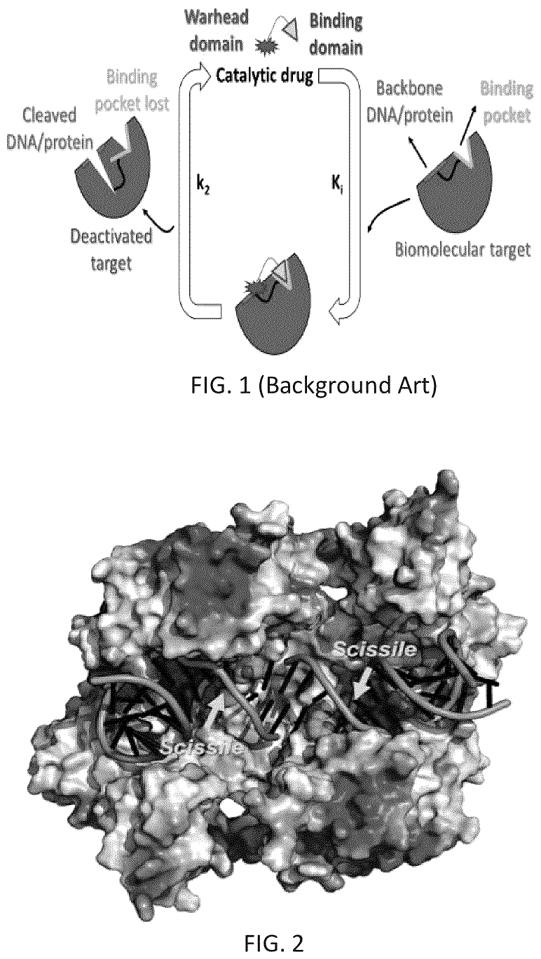

The present inventors have envisioned that such modified fluoroquinolones (i) irreversibly deactivate the enzyme target, (ii) fragment the chromosome, and (iii) interfere with the affinity of the binding moiety to the target such that a catalytic cycle is generated, as shown in Background Art FIG. 1.

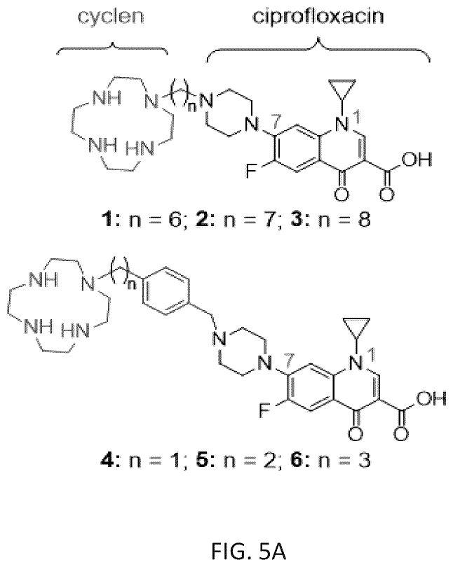

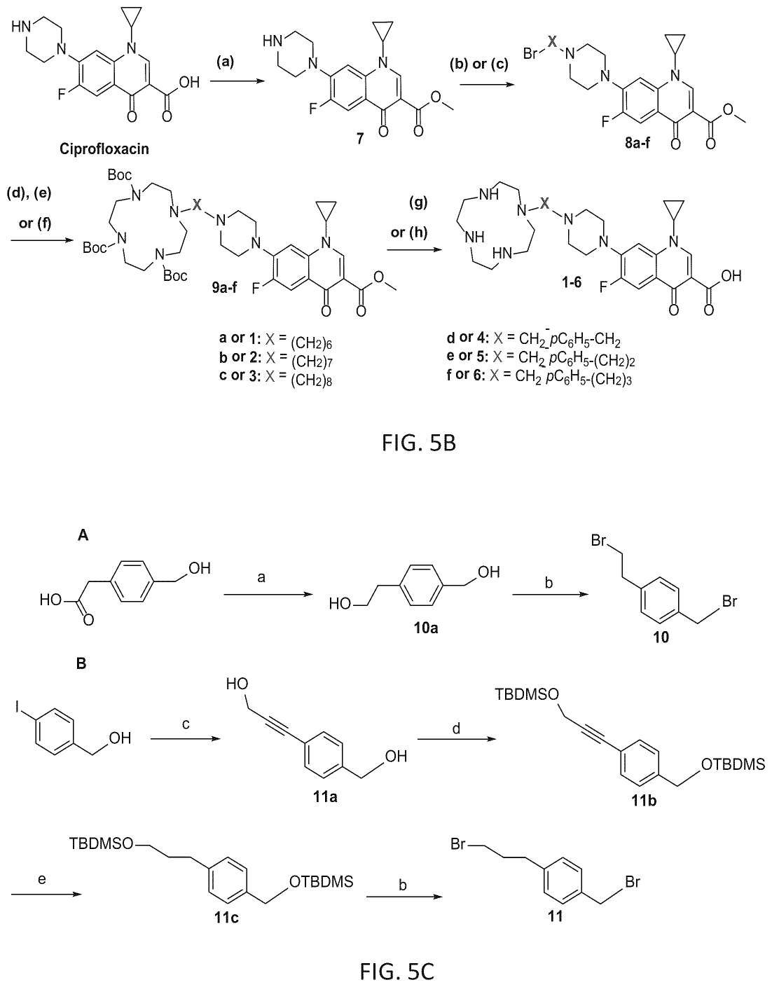

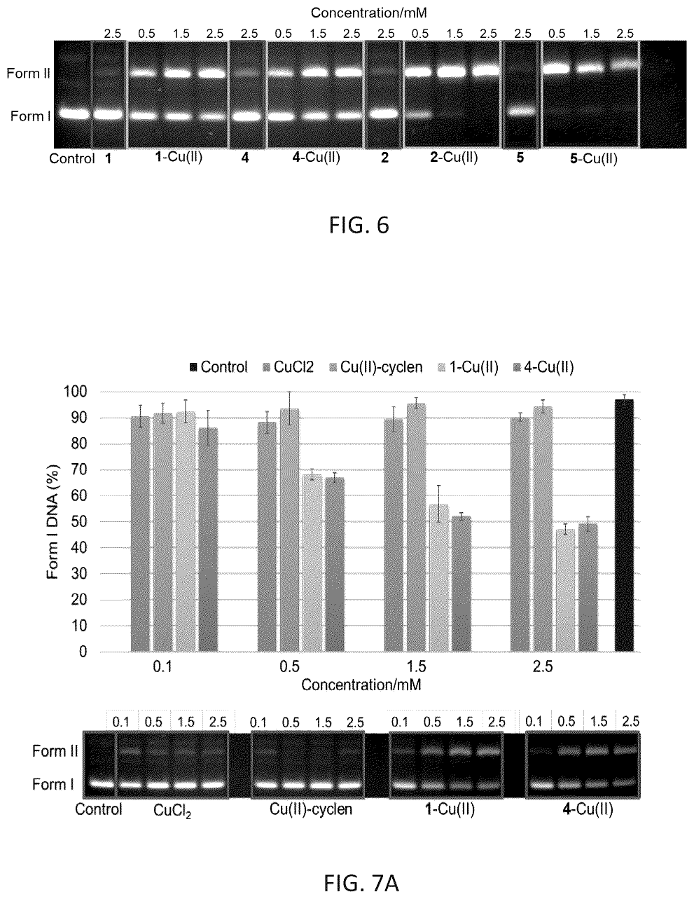

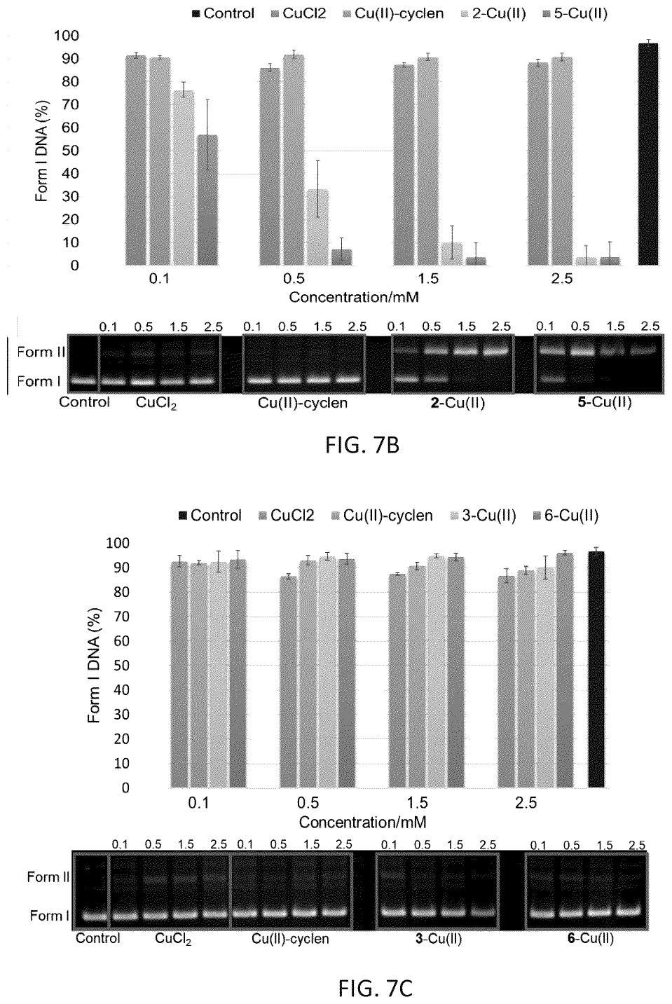

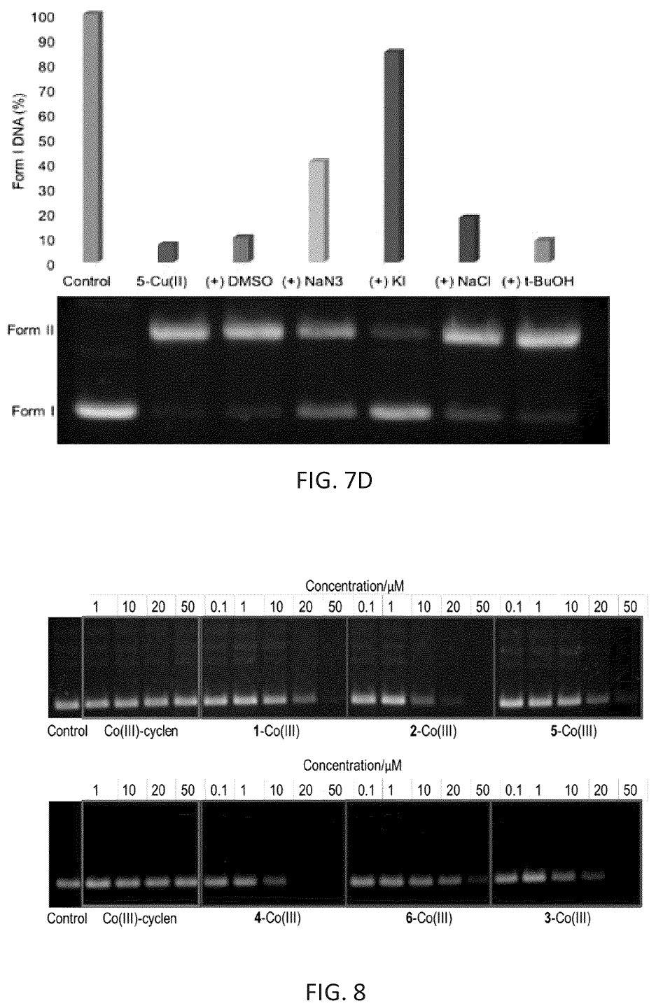

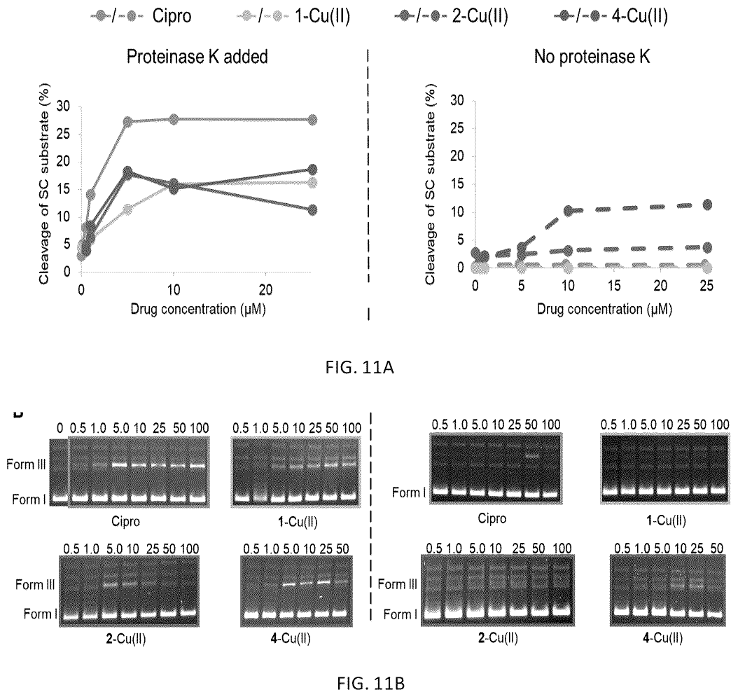

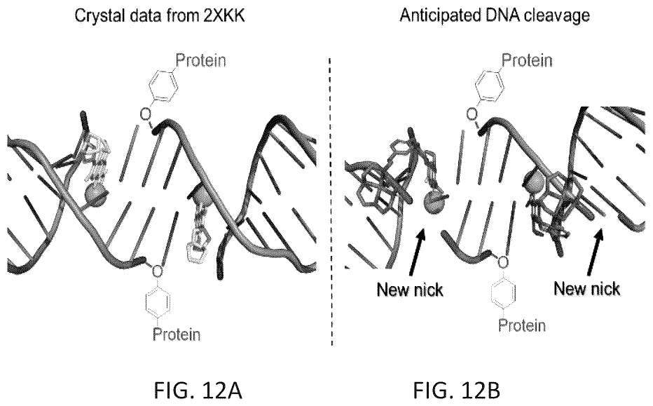

While reducing the present invention to practice, an initial library of ciprofloxacin-nuclease conjugates was generated (see, FIGS. 5A-C) and their potential as catalytic antibiotics was tested. As described in further detail in the Examples section that follows, metal (e.g., Cu(II)) complexes of the newly designed compounds showed excellent in vitro hydrolytic and oxidative DNase activity (see, FIGS. 6 and 7A-D), good antibacterial activity against both Gram-negative and Gram-positive bacteria (see, Table 2), and proved to be highly potent bacterial DNA gyrase inhibitors via a mechanism that involves stabilization of the ternary complex (see, FIGS. 12A and 12B). The metal (e.g., Cu(II)) complexes of the tested compounds were shown to fragment supercoiled plasmid DNA into linear DNA in the presence of DNA gyrase (see, FIGS. 11A and 11B).

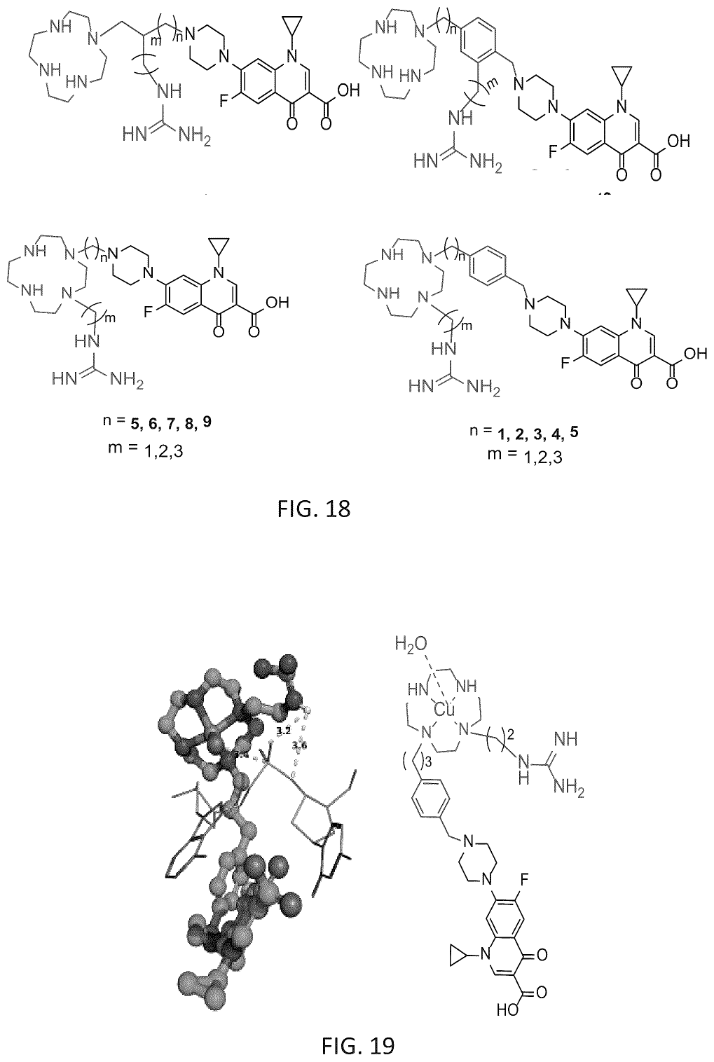

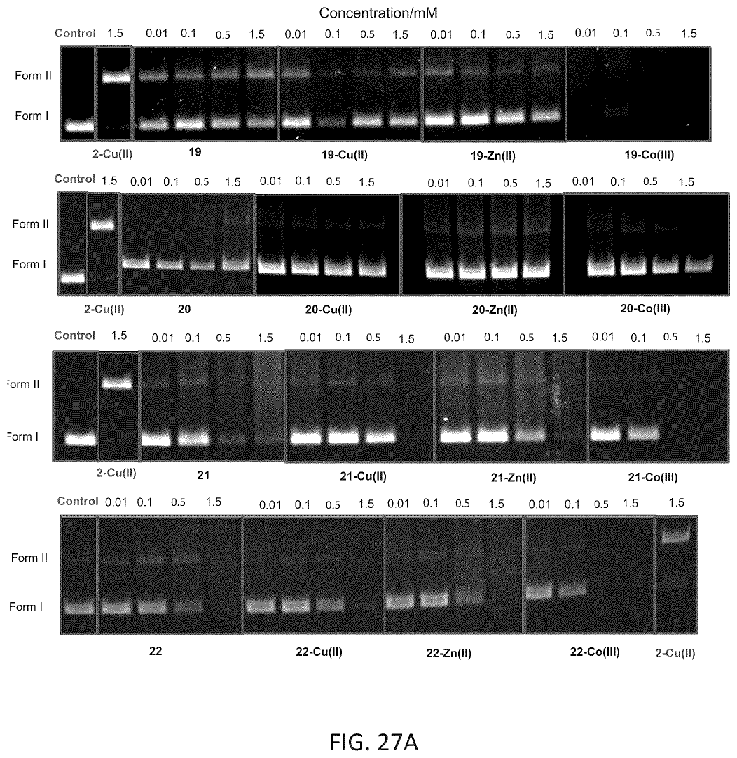

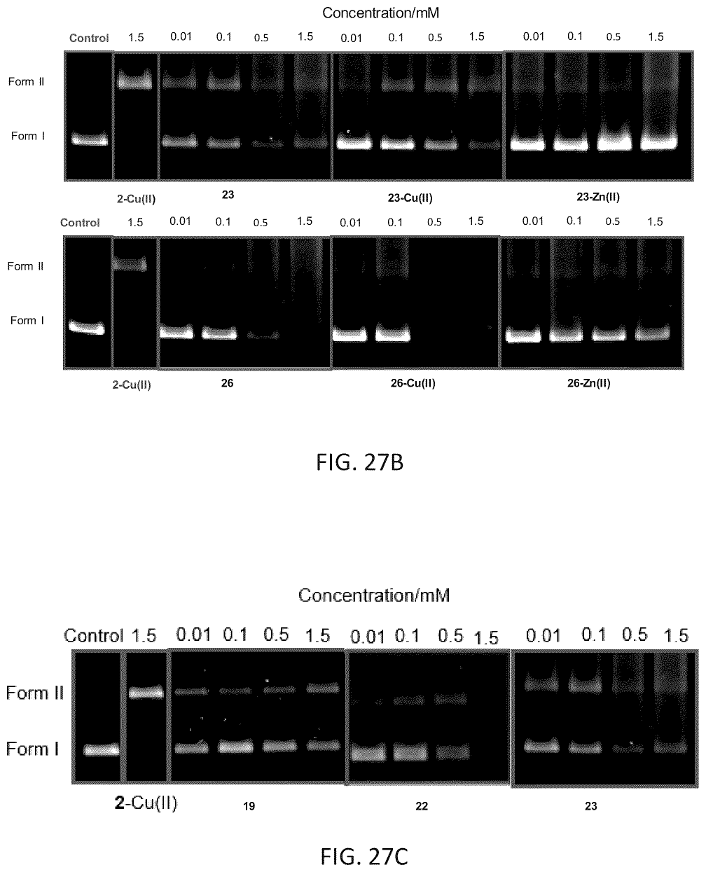

While some of the newly designed metal complexes were found to be vulnerable to cellular and other physiological components, a second generation of compounds, bearing guanidine and amine pendant moieties, and Cu(II), Zn(II) and Co(III) complexes thereof, were designed and successfully prepared. See, for example, FIGS. 18, 19, 20A-B, 21A-B, 23A-B, and 24A-C. As described in further detail in the Example section that follows, further insights were gained with regard to preferred structural features that impart to the compounds and the metal complexes thereof improved functionality. See, for example, FIGS. 27A-B, 29A-B, 30, 31A-B and 32A-B. All the additionally designed compounds and metal complexes exhibited good antibacterial activity against both Gram-negative and Gram-positive bacteria in the absence or presence of DTT (dithiothreitol; see, Tables 2 and 3).

Embodiments of the present invention relate to newly designed fluoroquinolone derivatives, to metal complexes thereof and to uses thereof in the treatment of conditions associated with pathogenic microorganisms.

Compound:

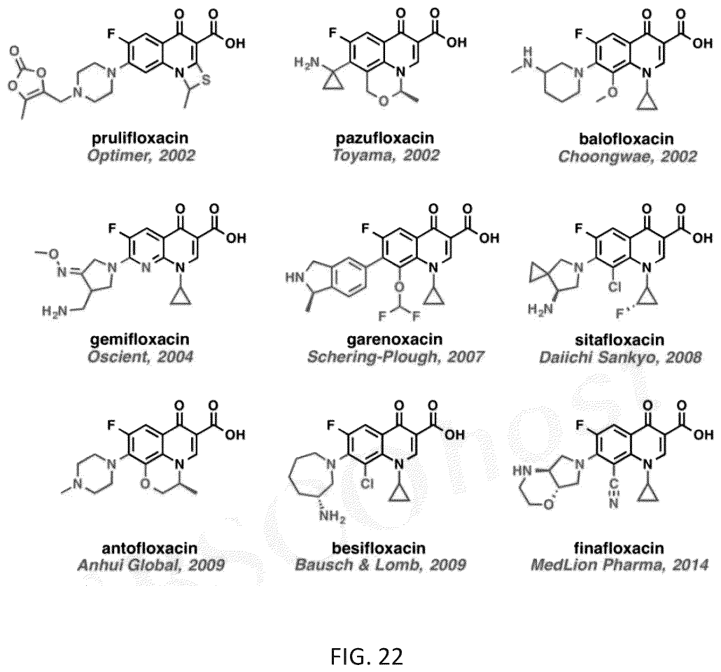

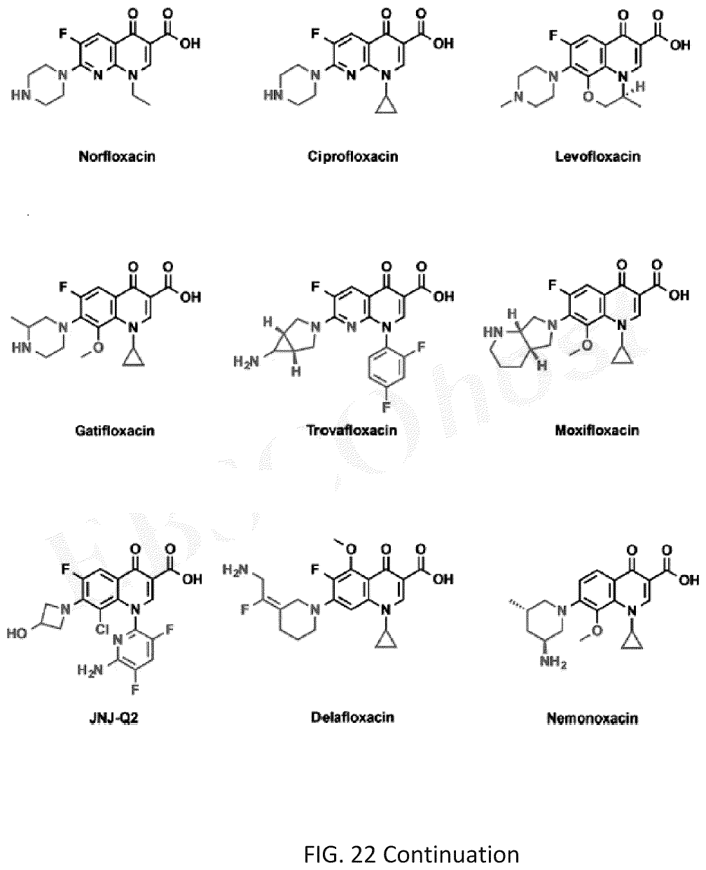

According to embodiments of the present invention there are provided compounds, which are modified quinolones, that is, quinolones to which a functional moiety that is aimed at acting as a nuclease is conjugated. These compounds can act as described herein per se, or can act as ligands for complexing therewith a metal ion, as described herein. These compounds, which can be collectively represented by Formula IV or Formula I, are also referred to herein as ligands, or as modified fluoroquinolones. In some embodiments, the quinolone portion of the compounds can be any of the quinolone structures presented in FIG. 22, or any other quinolone structure that exhibits an antibacterial activity.

According an aspect of some embodiments of the present invention there is provided a compound represented by Formula IV:

W-L-A-F Formula IV

Wherein:
W is a heteroalicyclic or a heteroaliphatic (a hydrocarbon containing one or more heteroatoms in its backbone or as pendant, substituent, groups) metal chelating moiety, and is preferably such that when having a metal ion chelated therewith, is capable of acting as DNA nuclease (capable of cleaving DNA);
L is a linking moiety (a linker) being of 6, 7 or 8 carbon atoms, preferably 6 or 7 carbon atoms, in length, which can be aliphatic or aromatic;
A is or comprises a heterocyclic moiety (heteroaryl or heteroalicyclic), or is or comprises a cycloalkyl substituted by an amine, and is preferably heteroalicyclic, more preferably an amine-containing heteroalicyclic (e.g., piperazine); and
F is a fluoroquinolone moiety, namely, a fluoroquinolone structure that is derived from known fluoroquinolone antibiotics, such as, but not limited to, described in FIG. 22.

Compounds of Formula IV can be regarded as conjugates in which a fluoroquinolone moiety is coupled to the W moiety via the L linking moiety, whereby the moiety A can be either part of the fluoroquinolone antibiotic, such that W is coupled via L to F-A, via one or more positions on the A portion of the fluoroquinolone, or whereby A forms a part of the moiety that links a fluoroquinolone skeleton to W, and A is attached to the fluoroquinolone skeleton preferably at a position ortho to the fluoro substituent. For the latter, the fluoroquinolone skeleton can be any of the fluoroquinolone portions of the fluoroquinolone antibiotics such as shown in FIG. 22.

Alternatively, F, or F-A, is a moiety that is derived from an antibiotic that targets a bacterial nucleic acid. The antibiotic can be a fluoroquinolone or any other antibiotic that acts by binding to, or interfering with an interaction of, a nucleic acid such as DNA, RNA and any other, as described herein.

According to some embodiments of the present invention, compounds of Formula IV can be represented by Formula I:

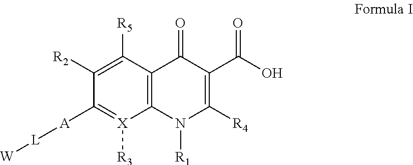

Wherein:
X is C or N, wherein when X is C the dashed line represents a bond and when X is N R₃ is absent;
R₁ is hydrogen, alkyl, aryl, heteroaryl or cycloalkyl, or alternatively, R₁ and R₄ or R₁ and R₃ form together a heterocyclic (heteroaryl or heteroalicyclic) ring, whereby R₁ is preferably a cycloalkyl (e.g., cyclopropyl);
R₂ is hydrogen or halo and is preferably fluoro;
R₃, if present, is hydrogen, alkyl, halo, alkoxy, thioalkoxy, aryloxy, thioaryloxy, aryl, heteroaryl, cyano (nitrile) or, alternatively, forms with R₁ a heterocyclic (heteroaryl or heteroalicyclic) ring;
R₄ is hydrogen, alkyl, cycloalkyl, or halo, or, alternatively, forms with R₁ the heterocyclic (heteroaryl or heteroalicyclic) ring;
R₅ is hydrogen, alkyl or cycloalkyl;
A is or comprises a heterocyclic moiety (heteroaryl or heteroalicyclic), or is or comprises a cycloalkyl substituted by an amine, and is preferably heteroalicyclic, more preferably an amine-containing heteroalicyclic (e.g., piperazine);
L is a linking moiety (a linker) being of 6, 7 or 8 carbon atoms, preferably 6 or 7 carbon atoms, in length, which can be aliphatic or aromatic (e.g., comprises an aromatic moiety such as aryl or heteroaryl as defined herein either as a pendant group or within the linking moiety itself); and
W is a heteroalicyclic or a heteroaliphatic (a hydrocarbon containing one or more heteroatoms in its backbone or as pendant, substituent, groups) metal chelating moiety, and is preferably such that when having a metal ion chelated therewithin or associated therewith, is capable of acting as DNA nuclease (capable of cleaving DNA).

According to some of any of the embodiments described herein, R₁ is alkyl or cycloalkyl and is preferably a cycloalkyl such as cyclopropyl. Any other cycloalkyl or heteroalicyclic groups, as defined herein, are contemplated.

According to some of any of the embodiments described herein, X is C and R₃ is present and can be hydrogen, or any of the substituents as defined for this variable.

According to some of any of the embodiments described herein, R₁ is cycloalkyl such as cyclopropyl, X is C and R₃ is present and can be hydrogen, or any of the substituents as defined for this variable.

In alternative embodiments, X is C and R₁ and R₃ form together a heteroalicyclic or heteroaryl ring.

According to some of any of the embodiments described herein, X is N.

According to some of any of the embodiments described herein, R₂ is halo, and is preferably fluoro.

According to some of any of the embodiments described herein, R₄ and R₅ are each hydrogen.

The moiety A can be any heterocyclic, preferably heteroalicyclic moiety, more preferably nitrogen-containing heteroalicyclic moiety, which can feature one, two or three rings, each can be substituted or unsubstituted, and each can be 4-, 5-, 6-, 7-, or 8-membered ring. Higher tings are also contemplated.

According to some of any of the embodiments described herein for Formula I or IV, A is a piperazine, which can be substituted or unsubstituted, and in some embodiments A is unsubstituted piperazine.

According to some of any of the embodiments described herein for Formula I, R₁-R₅, X and A are such that provide or correspond to ciprofloxacin. See, for example, FIGS. 5A, 23A, 24A-B and 33A-B.

According to some of any of the embodiments described herein for Formula I, R₁-R₅, X and A are such that provide or correspond to an antibacterial quinolone as shown in FIG. 22, or any other quinolone that exhibits an antibacterial activity.

According to some of any of the embodiments described herein for Formula I or IV, the linker L is aliphatic or aromatic.

According to some of any of the embodiments described herein for Formula I or IV, the linker L is aliphatic, that is, it is or comprises a non-aromatic hydrocarbon chain, of 6, 7, 8 or 9, or of 6, 7 or 8, atoms in length. In some of these embodiments, the hydrocarbon chain is an aliphatic, linear (non-branched) hydrocarbon chain, for example, an alkylene chain.

According to some of any of the embodiments described herein for Formula I or IV, L is an alkylene chain of 6 or 7 carbon atoms in length, preferably of 7 carbon atoms in length.

The alkylene can be substituted or unsubstituted. In some embodiments it is unsubstituted.

According to some of any of the embodiments described herein, L is an alkylene chain of 6 or 7 carbon atoms in length, and one or more carbon atoms in the alkylene chain is substituted a moiety P as described herein. According to some of these embodiments, at least one carbon atom to which moiety P is attached is separated from the metal chelating moiety W by 0, 1 or 2 carbon atoms, preferably 0 or 1 carbon atoms. According to some of any of the embodiments described herein for Formula I or IV, the linker L is aromatic, that is, it comprises one or more aryl or heteroaryl in its chain or as substituent(s) of one or more atoms in the linker chain.

According to some of any of the embodiments described herein for Formula I or IV, L is aromatic and includes an aryl (e.g., phenyl) in its chain. According to some of these embodiments, L is 6, 7, 8, 9 or 10, preferably 6 or 7, more preferably 6, carbon atoms in length. In some embodiments, when the aryl is phenyl, it is considered as being 4 carbon atoms in length within the linker L, and the remaining atoms are preferably carbon atoms, for example, one or alkylene chains between the phenyl and A and/or one or more alkylene chains between the phenyl and W.

These alkylene chains can be substituted or unsubstituted, as described herein for an aliphatic linker L.

According to some of any of the embodiments described herein, L is —(CRaRb)-Aryl-(CRcRd)-, or —(CRaRb)-Aryl-(CRcRd)-(CReRf)—, for example, (CRaRb)-Ph-(CRcRd) or —(CRaRb)-Ph-(CRcRd)-(CReRf)—, wherein Ra-Rd, and Re and Rf, if present, are each independently hydrogen, alkyl or a moiety P as described herein.

According to some of any of the embodiments described herein for Formula I or IV, L is (CRaRb)-Aryl-(CRcRd), for example, (CRaRb)-Ph-(CRcRd).

According to some of any of the embodiments described herein, each of Ra-Rd, and Re and Rf, if present, is hydrogen.

According to some of any of the embodiments described herein, the aryl (e.g., the phenyl) in an aromatic linker is unsubstituted or is substituted by one or more of halo, alkyl, cycloalkyl and a moiety P as described herein.

According to some of any of the embodiments described herein, the aryl (e.g., phenyl) is substituted by the moiety P. According to some of these embodiments, the moiety P is a substituent at a distal position from the moiety W.

According to some of any of the embodiments described herein, at least one of Rc and Rd, and Re and Rf, if present, is the moiety P. Preferably, the moiety P is attached to the aryl (e.g., phenyl group) or to a carbon atom in the linker that is spaced apart from the attachment point to W by 0, 1, 2, or 3 carbon atoms.

According to some of any of the embodiments described herein for Formula I or IV, the moiety W, which is conjugated to the fluoroquinolone, or to F, via the linker L, preferably serves as a DNA nuclease, that is, it is capable of inducing DNA cleavage. Preferably, it is a moiety that, when associated with a metal ion, is capable of inducing cleavage of a bacterial DNA. Further preferably, it is capable of inducing catalytic DNA cleavage. The moiety W can alternatively be a metal chelating moiety that when associated with a metal ion acts, reversibly or irreversibly, and optionally catalytically, by interfering with the functionality of a nucleic acid that is targeted by the fluoroquinolone, or by F in Formula I.

According to some of any of the embodiments described herein, W is a heteroaliphatic moiety, that is, it is a hydrocarbon chain (linear or branched) that comprises one or more, preferably two or more, heteroatoms in its chain and/or as pendant, substituent, groups. The heteroatoms are preferably such that can coordinate the metal ion, and are spatially arranged in a proximity and orientation that enables efficient coordination with the metal ion. According to some of these embodiments, W is capable of acting as a nuclease (capable of cleaving a nucleic acid, as described herein). An exemplary heteroaliphatic moiety is an alkylene chain, for example, of 1, 2, 3 or 4 carbon atoms, that is terminated by two guanidine groups, or any other heteroatom-containing groups that can coordinate with the metal ion (e.g., as described herein for a group Z). The alkylene chain is linked to linker L via one of the carbon atoms thereof.

According to some of any of the embodiments described herein, W is a heteroalicyclic moiety that comprises at least one, preferably at least two, heteroatoms within the cyclic ring. The heteroatoms can be, for example, nitrogen (amine), oxygen, and/or sulfur, preferably, nitrogen (amine) and/or oxygen. The amine can be secondary or tertiary and is preferably secondary. According to some of any of the embodiments described herein, W is a heteroalicyclic moiety that comprises at least two nitrogen heteroatoms (two amine groups, preferably two secondary amine groups).

Exemplary heteroalicyclic metal chelating moieties W include, but are not limited to:

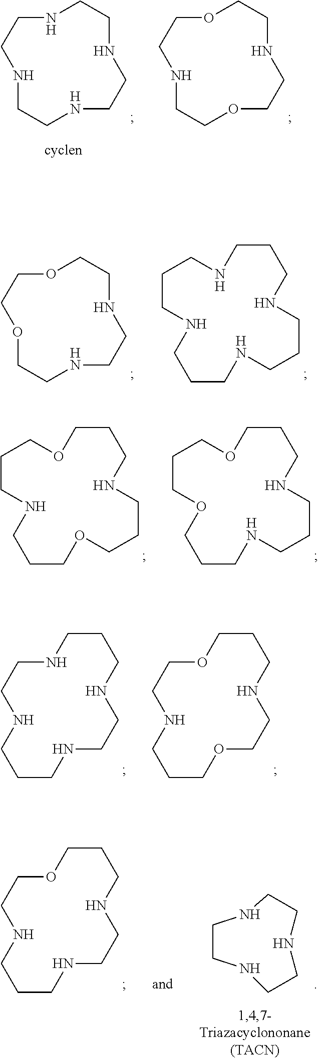

According to some of any of the embodiments described herein, W is cyclen.

According to some of any of the embodiments described herein for Formula I or IV, the W metal chelating moiety is substituted or unsubstituted. When substituted, the substituent can be any of the substituents described herein for a heteroalicyclic or aliphatic group, as long as the substituent does not interfere with the metal chelating function of the W moiety.

According to some embodiments, W is an unsubstituted moiety.

According to some embodiments, W is substituted by one or more of moiety P as described herein.

According to some of these embodiments, the moiety P is attached to a heteroatom in the heteroalicyclic chelating moiety which is ortho to the attachment point to the L. By “ortho” it is meant the first heteroatom in the cyclic moiety that is adjacent to the attachment point to L.

According to some of any of the embodiments described herein for Formula IV or I, the compound comprises at least one moiety that comprises a heteroatom-containing group that is at a non-protonated or not fully protonated form at a physiological pH, namely, has pKa lower than 8, preferably lower than 7, more preferably, lower than 6 (e.g., a moiety P as described herein).

According to some of any of the embodiments described herein for Formula IV or I, the compound comprises at least one moiety that comprises a heteroatom-containing group that is capable of reversibly binding to the metal ion (e.g., in physiological environment) (e.g., a moiety P as described herein).

According to some of any of the embodiments described herein for Formula IV or I, the compound comprises at least one moiety that comprises a heteroatom-containing group that is at a non-protonated or not fully protonated form at a physiological pH, namely, has pKa lower than 8, preferably lower than 7, more preferably, lower than 6 and/or is capable of reversibly binding to the metal ion (e.g., in physiological environment) (e.g., a moiety P as described herein).

According to some of any of the embodiments described herein for Formula IV or I, the compound comprises at least one moiety that comprises a heteroatom-containing group that is at a non-protonated or not fully protonated form at a physiological pH, namely, has pKa lower than 8, preferably lower than 7, more preferably, lower than 6 and/or is capable of reversibly binding to the metal ion (e.g., in physiological environment) (e.g., a moiety P as described herein).

A moiety that comprises a heteroatom-containing group as described herein in any of the respective embodiments is also referred to herein as moiety P.

Such as moiety is also referred to herein as a “protecting” moiety (see, the Examples section that follows).

Herein throughout, a heteroatom-containing group of moiety P is also referred to herein as group Z, and can include one or more heteroatoms such as N, O, S, etc., as long as a reversible, non-covalent, binding with the metal is enabled. In some embodiments, the heteroatom is nitrogen, and the heteroatom-containing group is an amine- or polyamine-containing group (e.g., guanidine), as long as at least one amine group is not protonated or not fully protonated at physiological pH, as described herein.

According to some of any of the embodiments described herein for Formula IV or I, the moiety that comprises a heteroatom-containing group, moiety P, is attached to the linker L and/or to the metal chelating moiety W. In some of any of these embodiments, moiety P is attached to a position of the linker and/or the metal chelating moiety that allows it to reversibly interact with the chelated metal ion.

According to some of any of the embodiments described herein for Formula IV or I, the moiety that comprises a heteroatom-containing group, moiety P, is attached to the linker L.

According to some of these embodiments, the linker L is a non-aromatic hydrocarbon chain as described herein, and one or more of moiety P is/are attached to one or more carbon atom(s) of the hydrocarbon chain, as described herein.

According to some of these embodiments, the linker L is or comprises an alkylene chain as described herein and the one or more of moiety P is/are attached to one or more carbon atom(s) of the alkylene chain, as described herein.

According to some of these embodiments, a carbon atom in the alkylene chain that is substituted by a moiety P is separated from the chelating moiety W (from the attachment point of linker L to W) by 0, 1 or 2 carbon atoms, preferably 0 or 1 carbon atoms.

According to some of any of the embodiments described herein, L is an aromatic linker which comprises at least one aryl, preferably phenyl, and the aryl or phenyl is substituted by one or more of moiety P, as described herein.

According to some of any of these embodiments, there are provided compounds that are collectively represented by Formula IVa or IVb:

W-L₁-A-F Formula IVa

W₁-L-A-F Formula IVb

W₁-L₁-A-F Formula IVc
wherein W, L, A and F are as described herein for Formula IV in any of the respective embodiments and any combination thereof, and L₁ and W₁ are as defined hereinbelow.

According to some of any of the embodiments of Formula IVa, IVb or IVc, F, or F-A is derived from any antibiotic that targets a nucleic acid or that its effect can be enhanced by nucleic acid cleavage, as described herein.

According to some of any of the embodiments of Formula IVa, IVb or IVc, F is a quinolone, such as fluoroquinolone, and F or F-A can be derived from any of the antibiotics shown in FIG. 22.

According to some of any of the embodiments of Formula IVa, IVb or IVc, F is a quinolone, for example, a fluoroquinolone, and the compounds can be collectively represented by Formula Ia, Ib or Ic:

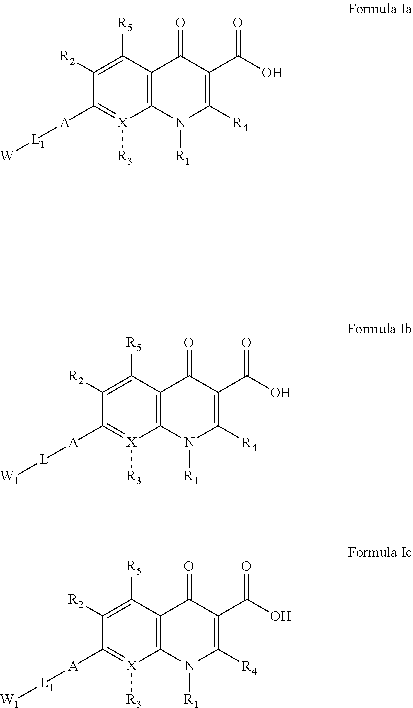

wherein:
R₁, R₂, R₃, R₄, R₅, A, L and W are as defined for Formula I in any of the respective embodiments and any combination thereof, and L₁ and W₁ are as defined hereinbelow.

According to some of any of the embodiments described herein for Formula IVa, IVc, Ia and Ic, L₁ is a linker as described herein, which comprises one or more of a moiety that comprises the heteroatom-containing group, that is one or more of a moiety P, as described herein in any of the respective embodiments and any combination thereof.

According to some of these embodiments, L₁ is a non-aromatic hydrocarbon as described herein in any of the respective embodiments for variable L in Formula IV or I, and moiety P is attached to one of the carbon atoms of the hydrocarbon.

According to some of these embodiments, L₁ is an alkylene chain of 6 or 7 carbon atoms in length, and at least one carbon of the alkylene chain is substituted by the moiety that comprises the heteroatom-containing group, moiety P, as described herein in any of the respective embodiments.

According to some of any of the embodiments described herein for Formula IVa and Ia, L₁ is an aromatic linker that comprises at least one aryl, as described herein in any of the respective embodiments for variable L in Formula IV or I.

According to these embodiments, the aryl, or one or more carbon atoms that are attached to the aryl within the linker L₁, is substituted by the moiety that comprises the heteroatom-containing group, moiety P, as described herein in any of the respective embodiments for variable L in Formula IV or I.

According to some of any of the embodiments described herein for Formula IVa, IVc, Ia and Ic, L₁ is —(CRaRb)-Aryl-(CRcRd)-, or —(CRaRb)-Aryl-(CRcRd)-(CReRf)—, wherein Ra-Rd, and Re and Rf, if present, are each independently hydrogen, alkyl or the moiety that comprises the heteroatom-containing group, moiety P, and the Aryl (e.g., phenyl) is substituted by the moiety that comprises the heteroatom-containing group, moiety P.

According to some of these embodiments, each of Ra-Rd, and Re or Rf, if present, is hydrogen.

According to some of any of the embodiments described herein for Formula IVa, IVc, Ia and Ic, L₁ is —(CRaRb)-Aryl-(CRcRd)-, or —(CRaRb)-Aryl-(CRcRd)-(CReRf)—, wherein Ra-Rd, and Re and Rf, if present, are each independently hydrogen, alkyl or the moiety that comprises the heteroatom-containing group, moiety P, and at least one of the Ra-Rd, and Re and Rf, if present, is the moiety that comprises the heteroatom-containing group, moiety P. According to some of these embodiments, the Aryl is unsubstituted or is substituted by one or more of halo, alkyl, cycloalkyl and the moiety that comprises the heteroatom-containing group (moiety P).

Linker L₁ is therefore a linker L as described herein to which is/are attached one or more of moiety P as described herein in any of the respective embodiments and any combination thereof.

According to the embodiments of Formula IVb, IVc, Ib and Ic, W₁ is a metal chelating moiety, as described herein in any of the respective embodiments of Formula IV or I, and any combination thereof, preferably a heteroalicyclic metal chelating moiety as described herein, that is substituted by one or more of the moiety that comprises the heteroatom-containing group, moiety P, as described herein in any of the respective embodiments and any combination thereof.

According to some of any of the embodiments described herein, the heteroatom-containing group Z in the moiety P is attached directly to the respective position in the compound of Formula I, IV, Ia, Ib, Ic, IVa, IVb, or IVc. Preferably, it is attached via a linker (such that moiety P comprises the group Z and a linker). The linker can be a hydrocarbon chain, as defined herein, of from 1 to 8 atoms in length, e.g., 1 to 8 carbon atoms in length, preferably, 1 to 6 atoms, or 1 to 4 atoms, or 1 to 3 atoms, or 1 or 2 atoms (e.g., carbon atoms). According to these embodiments, the hydrocarbon chain is terminated by and/or substituted by the heteroatom-containing group Z, as defined herein. According to some of any of these embodiments, the linker in moiety P is an alkylene chain, which can be substituted or unsubstituted, of 1 to 6, or 1 to 4, preferably 1 to 3, carbon atoms, that terminates by the group Z.

It is to be noted that the length of the linker in moiety P and the position to which moiety P is attached can be manipulated so as to provide the best reversible interaction between the group Z and a metal ion when associated with W.

According to some of any of the embodiments described herein for Formula Ia, Ib, Ic, IVa, IVb, or IVc, moiety P comprises an alkylene chain, as defined herein, of from 1 to 3 carbon atoms in length that is terminated by the heteroatom-containing group Z, as defined herein.

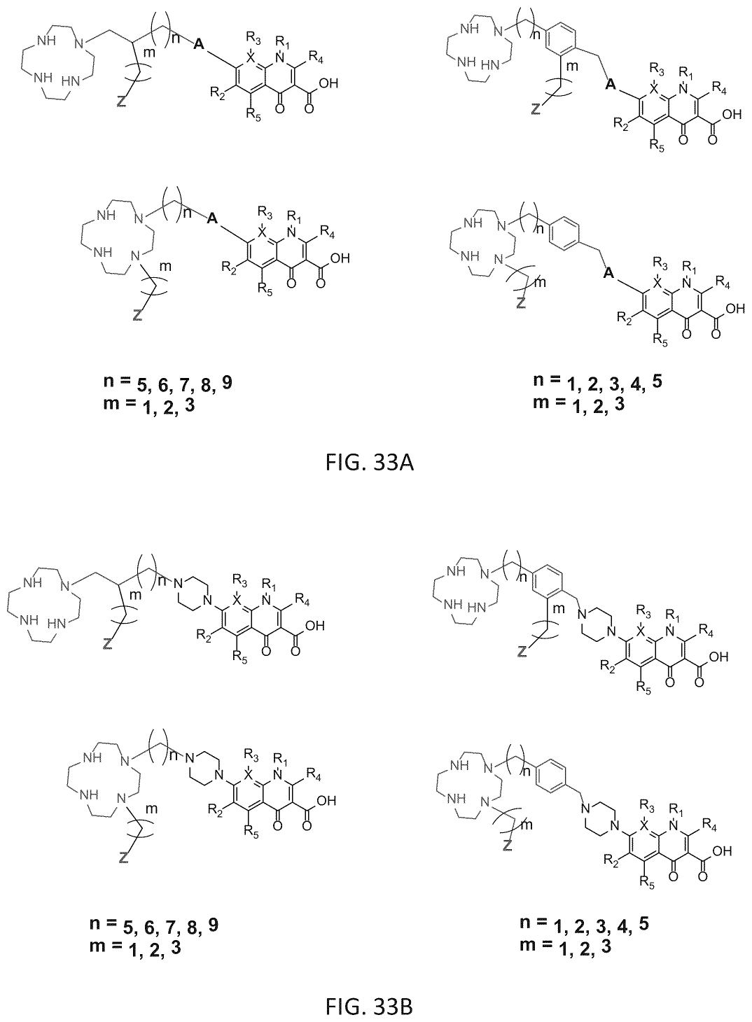

Exemplary such compounds of Formula Ia and Ib are presented in FIG. 33A. The right structures are of compounds in which L₁ or L is an aromatic linker, and the left structures are compounds in which L₁ or L is an aliphatic linear linker. The upper structures are for compounds in which moiety P is attached to the linker and the lower structures are for compounds in which P is attached to the metal chelating moiety W, exemplified as cyclen in FIG. 33A.

FIG. 33B presents exemplary such compounds of Formula Ia and Ib in which moiety A is piperazine. The right structures are of compounds in which L₁ or L is an aromatic linker, and the left structures are compounds in which L₁ or L is an aliphatic linear linker. The upper structures are for compounds in which moiety P is attached to the linker and the lower structures are for compounds in which P is attached to the metal chelating moiety W, exemplified as cyclen.

According to some of any of the embodiments described herein for moiety P and compounds containing one or more of moiety P, the heteroatom-containing group Z is guanidine.

According to some of any of the embodiments described herein for moiety P and compounds containing one or more of moiety P, the heteroatom-containing group Z is an amine, preferably a secondary or primary amine, and more preferably it is a primary amine.

Compounds according to these embodiments can be collectively represented by Formula Va, Vb or Vc:

W-L₂-A-F Formula Va

W₂-L-A-F Formula Vb

W₂-L₂-A-F Formula Vc
wherein W, L, A and F are as described herein for Formula IV in any of the respective embodiments and any combination thereof; L₂ is as defined herein for L₁ in any of the respective embodiments and any combination thereof, wherein the moiety P comprises a the heteroatom-containing group Z which is an amine, preferably a primary amine; and W₂ is as defined herein for W₁ in any of the respective embodiments and any combination thereof, wherein the moiety P comprises a the heteroatom-containing group Z which is an amine, preferably a primary amine.

According to some of any of the embodiments of Formula Va, Vb or Vc, F, or F-A is derived from any antibiotic that targets a nucleic acid or that its effect can be enhanced by nucleic acid cleavage, as described herein.

According to some of any of the embodiments of Formula Va, Vb or Vc, F is a quinolone, such as fluoroquinolone, and F or F-A can be derived from any of the antibiotics shown in FIG. 22.

According to some of any of the embodiments of Formula Va, Vb or Vc, F is a quinolone, for example, a fluoroquinolone, and the compounds can be collectively represented by Formula IIa, IIb or IIc:

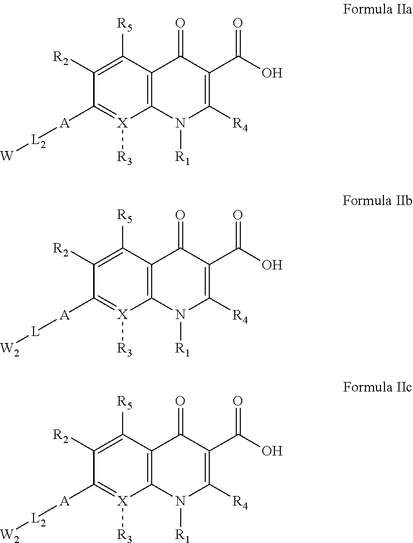

wherein:
R₁, R₂, R₃, R₄, R₅, A, L and W are as defined for Formula I in any of the respective embodiments and any combination thereof; L₂ is as defined herein for L₁ in any of the respective embodiments and any combination thereof, wherein the moiety P comprises a the heteroatom-containing group Z which is an amine, preferably a primary amine; and W₂ is as defined herein for W₁ in any of the respective embodiments and any combination thereof, wherein the moiety P comprises a the heteroatom-containing group Z which is an amine, preferably a primary amine.

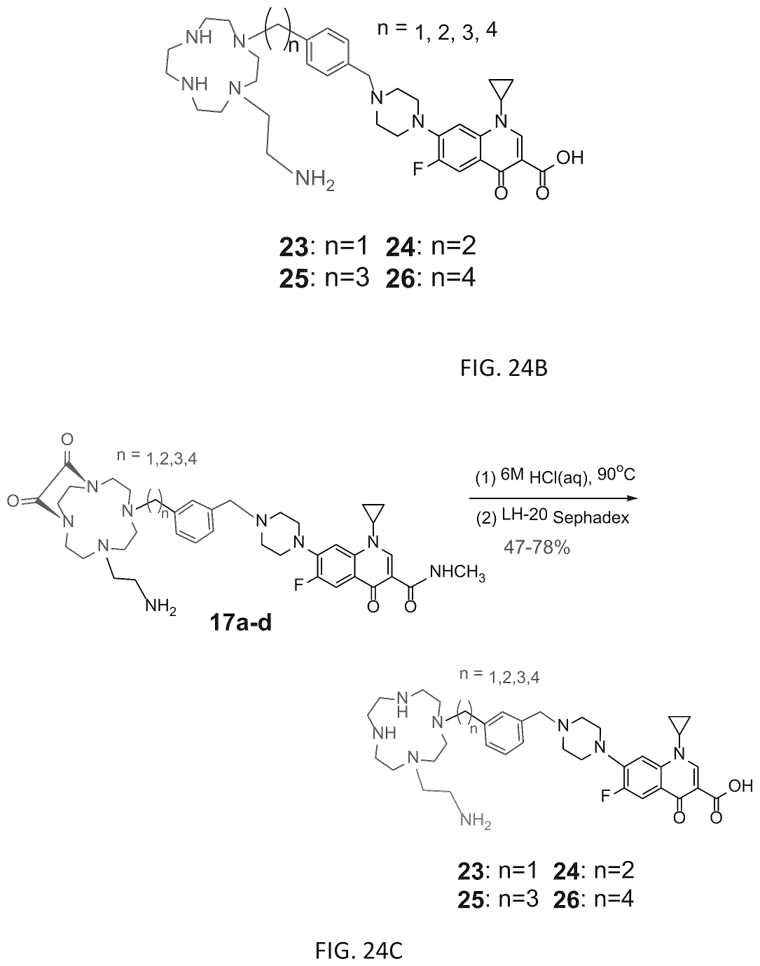

Exemplary such compounds are presented in FIGS. 33A-B, wherein Z is a primary amine; and in FIGS. 24A and 24B.

Herein throughout, the moiety P as described herein in any of the respective embodiments is also referred to as “pendant”.

According to further aspects of some embodiments of the present invention there are provided processes of preparing any of the compounds as described herein and any intermediates thereof, which are essentially as described herein in the Examples section that follows.

According to further aspects of some embodiments of the present invention there are provided compounds as described herein as intermediates en route of obtaining the fluoroquinolone compounds as described herein. Such intermediates are essentially as described herein in the Examples section that follows and accompanying figures.

Metal Complexes:

According to an aspect of some embodiments of the present invention there is provided a complex comprising the compound (ligand) as described herein in any of the respective embodiments (e.g., of Formula I, Ia, Ib, Ic, IIa, IIb, IIc, IV, IVa, IVb, IVc, Va, Vb or Vc) and a metal ion associated with the W metal chelating moiety.

According to some of any of the embodiments described herein, the metal is a redox reactive and/or Lewis acid metal.

The metal can be, for example, copper, cobalt, zinc, magnesium, iron, nickel and manganese.

According to some of any of the embodiments described herein, the metal ion is Cu(II).

According to some of any of the embodiments described herein, the metal ion is Zn(II).

According to some of any of the embodiments described herein, the metal ion is Co(III).

According to some of any of the embodiments described herein, the complex is capable of cleaving a bacterial DNA (e.g., via hydrolytic mechanism) and/or of interfering with bacterial DNA gyrase activity.

According to some of any of the embodiments described herein, the complex is capable of fragmenting bacterial supercoiled plasmid DNA into linear DNA.

According to some of any of the embodiments described herein, the complex is capable of reducing a population of a pathogenic microorganism (e.g., a bacterium), or otherwise affect the microorganism as described herein.

According to some of any of the embodiments described herein, the complex is capable of reducing the population of the microorganism as described herein in a catalytic matter.

According to an aspect of some embodiments of the present invention there is provided a complex comprising a compound of Formula I and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula Ia and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula Ib and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula Ic and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula IIa and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula IIb and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula IIc and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula IV and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula IVa and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula IVb and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula IVc and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula Va and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula Vb and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex comprising a compound of Formula Vc and a metal ion associated with the moiety W.

According to some of any of the embodiments of this aspect, the metal ion is Cu(II).

According to some of any of the embodiments of this aspect, the metal ion is Zn(II).

According to some of any of the embodiments of this aspect, the metal ion is Co(III).

According to an aspect of some embodiments of the present invention there is provided a complex of Formula I, Ia, Tb, Ic, IIa, IIb, IIc, IV, IVa, IVb, IVc, Va, Vb or Vc and a metal ion associated with the W metal chelating moiety, wherein the metal ion is Co(III).

According to an aspect of some embodiments of the present invention there is provided a complex of Formula Ta, Ib, Ic, IIa, IIb, IIc, IVa, IVb, IVc, Va, Vb or Vc and a metal ion associated with the W metal chelating moiety, wherein the metal ion is Co(III).

According to an aspect of some embodiments of the present invention there is provided a compound as described herein in any of the respective embodiments or a complex as described herein in any of the respective embodiments or a pharmaceutical composition as described herein, for use in the treatment a medical condition associated with a pathogenic microorganism.

According to some embodiments, the pathogenic microorganism is a bacterium.

As discussed herein, the present inventors have conceived and demonstrated that the introduction of a moiety P as described herein successfully and beneficially protects chelated metal ions from poisoning or otherwise inactivation by endogeneous (e.g., physiological, cellular) components.

Hence, according to an aspect of some embodiments of the present invention, there is provided a metal-containing therapeutically active agent that features a moiety P as described herein in any of the respective embodiments and any combination thereof. According to some embodiments of this aspect of the present invention, the moiety P is positioned in the respective compound such that it is capable of reversibly interacting with the metal ion (e.g., in physiological environment).

According to some embodiments of this aspect of the present invention, the metal-containing therapeutically active agent (a metallodrug) is such that targets a nucleic acid, for example, it features a metal chelating moiety such as described herein, and a metal ion associated therewith. The moiety P as described herein can be attached to the metal chelating moiety or to any other portion of the therapeutically active agent, as long as it has a chemical structure and position that enables its interaction with the metal ion, that is, it is spatially arranged in proximity and orientation with respect to the metal ion that enables its interaction therewith.

The metal-containing therapeutically active agent can be any such agent known in the art, which interferes with a functionality of a nucleic acid. The agent can act as a nuclease, which promotes cleavage of a nucleic acid, or such that interferes with a formation of complexes of a nucleic acid with other cellular components (e.g., proteins).

The nucleic acid can be, for example, DNA or RNA, including siRNA, micro RNA, mRNA, and guide RNA.

The term “nucleic acid” encompasses sequences of the naturally-occurring nucleobases and also encompasses sequences that include any of the known base analogs of DNA and RNA such as 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, uracil-5-oxyacetic acid, queosine, 2-thiocytosine and 2,6-diaminopurine.

In some embodiments, the nucleic acid is a plasmid.

I some embodiments, the nucleic acid is or comprises one or more nucleobase analogs such as, but not limited to, 4-acetylcytosine, 8-hydroxy-N6-methyladenosine, aziridinylcytosine, pseudo isocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydro uracil, inosine, N6-isopentenyladenine, 1-methyladenine, 1-methylpseudouracil, 1-methylguanine, 1-methylinosine, 2,2-dimethylguanine, 2-methyladenine, 2-methyl guanine, 3-methylcytosine, 5-methylcytosine, N6-methyladenine, 7-methylguanine, 5-methylaminomethyluracil, 5-methoxy-aminomethyl-2-thiouracil, 5′-methoxycarbonyl methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, uracil-5-oxyacetic acid, queosine, 2-thiocytosine and 2,6-diaminopurine.

Complexes according to some embodiments of this aspect of the present invention are usable, or for use, in the treatment of any medical condition that is treatable by the metallodrug.

Uses:

The compounds and/or complexes according to some embodiments of the present invention (e.g., compounds of Formula I, Ia, Ib, Ic, IIa, IIb, IIc, IV, IVa, IVb, IVc, Va, Vb or Vc, and metal complexes thereof as described herein in any of the respective embodiments and any combination therein) are effective in reducing a load of a pathogenic microorganism in or on a substrate.

The term “reducing the load” refers to a decrease in the number of the pathogenic microorganism(s), or to a decrease in the rate of their growth or both in the substrate as compared to a non-treated substrate.

The substrate can be an animate or non-animate substrate.

In some embodiments, the substrate is an animate substrate.

The compounds and/or complexes according to some embodiments of the present invention (e.g., compounds of Formula I, Ta, Ib, Ic, IIa, IIb, IIc, IV, IVa, IVb, IVc, Va, Vb or Vc, and metal complexes thereof as described herein in any of the respective embodiments and any combination therein) are effective in treating medical conditions associated with a pathogenic microorganism in a subject.

The compounds and/or complexes presented herein (e.g., compounds of Formula I, Ta, Ib, Ic, IIa, IIb, IIc, IV, IVa, IVb, IVc, Va, Vb or Vc, and metal complexes thereof as described herein in any of the respective embodiments and any combination therein) can also be effective in treating medical conditions associated with pathogenic microorganisms which have already developed resistance to an antibiotic agent (for example, a fluoroquinolone).

The phrases “effective in treating medical conditions associated with pathogenic microorganisms”, “effective in treating a subject diagnosed with a medical conditions associated with pathogenic microorganisms” and/or “for use in the treatment of a medical condition associated with a pathogenic microorganism in a subject”, as used herein interchangeably, refer to characteristics of a substance, such as the compounds and/or complexes according to some embodiments of the present invention, that can effect death, killing, eradication, elimination, reduction in number, reduction of growth rate, reduction of a load, and/or a change in population distribution of one or more species of pathogenic microorganisms, as well as effecting a reduction or prevention of the emergence of resistance of such microorganisms to the substance.

Herein throughout, the phrase “pathogenic microorganism” is used to describe any microorganism which can cause a disease or disorder in a higher organism, such as mammals in general and a human in particular. The pathogenic microorganism may belong to any family of organisms such as, but not limited to prokaryotic organisms, eubacterium, archaebacterium, eukaryotic organisms, yeast, fungi, algae, protozoan, and other parasites.

Non-limiting examples of pathogenic microorganism include Plasmodium falciparum: and related malaria-causing protozoan parasites, Acanthamoeba and other free-living amoebae, Aeromonas hydrophila, Anisakis and related worms, and further include, but not limited to Acinetobacter baumanii, Ascaris lumbricoides, Bacillus cereus, Brevundimonas diminuta, Campylobacter jejuni, Clostridium botulinum, Clostridium perfringens, Cryptosporidium parvum, Cyclospora cayetanensis, Diphyllobothrium, Entamoeba histolytica, certain strains of Escherichia coli, Eustrongylides, Giardia lamblia, Klebsiella pneumoniae, Listeria monocytogenes, Nanophyetus, Plesiomonas shigelloides, Proteus mirabilis, Pseudomonas aeruginosa, Salmonella, Serratia odorifera, Shigella, Staphylococcus aureus, Stenotrophomonas maltophilia, Streptococcus, Trichuris trichiura, Vibrio cholerae, Vibrio parahaemolyticus, Vibrio vulnificus and other vibrios, Yersinia enterocolitica, Yersinia pseudotuberculosis and Yersinia kristensenii.

Other pathogens include Strep. pyogenes (Group A), Strep. pneumoniae, Strep. GpB, Strep. viridans, Strep. GpD (Enterococcus), Strep. GpC and GpG, Staph. aureus, Staph. epidermidis, Bacillus subtilis, Bacillus anthracis, Listeria monocytogenes, Anaerobic cocci, Clostridium spp., Actinomyces spp, Escherichia coli, Enterobacter aerogenes, Kiebsiella pneumoniae, Proteus mirabilis, Proteus vulgaris, Morganella morganii, Providencia stuartii, Serratia marcescens, Citrobacter freundii, Salmonella typhi, Salmonella paratyphi, Salmonella typhi murium, Salmonella virchow, Shigella spp., Yersinia enterocolitica, Acinetobacter calcoaceticus, Flavobacterium spp., Haemophilus influenzae, Pseudomonas aeruginosa, Campylobacter jejuni, Vibrio parahaemolyticus, Brucella spp., Neisseria meningitidis, Neisseria gonorrhoea, Bacteroides fragilis, Fusobacterium spp., Mycobacterium tuberculosis (including MDR and XDR strains from hospital origins isolated from patients) and Mycobacterium smegmatis.

Accordingly, a condition associated with a pathogenic microorganism describes an infectious condition that results from the presence of the microorganism in a subject. The infectious condition can be, for example, a bacterial infection, a fungal infection, a protozoal infection, and the like, collectively referred to herein as “microbial infection”.

Some higher forms of microorganisms are pathogenic per-se, and other harbor lower forms of pathogenic bacteria, thus present a medical threat expressed in many medical conditions, such as, without limitation, actinomycosis, anthrax, aspergillosis, bacteremia, bacterial skin diseases, bartonella infections, botulism, brucellosis, burkholderia infections, campylobacter infections, candidiasis, cat-scratch disease, chlamydia infections, cholera, clostridium infections, coccidioidomycosis, cryptococcosis, dermatomycoses, dermatomycoses, diphtheria, ehrlichiosis, epidemic louse borne typhus, Escherichia coli infections, fusobacterium infections, gangrene, general infections, general mycoses, gram-negative bacterial infections, Gram-positive bacterial infections, histoplasmosis, impetigo, klebsiella infections, legionellosis, leprosy, leptospirosis, listeria infections, lyme disease, maduromycosis, melioidosis, mycobacterium infections, mycoplasma infections, necrotizing fasciitis, nocardia infections, onychomycosis, ornithosis, pneumococcal infections, pneumonia, pseudomonas infections, Q fever, rat-bite fever, relapsing fever, rheumatic fever, rickettsia infections, Rocky-mountain spotted fever, salmonella infections, scarlet fever, scrub typhus, sepsis, sexually transmitted bacterial diseases, staphylococcal infections, streptococcal infections, surgical site infection, tetanus, tick-borne diseases, tuberculosis, tularemia, typhoid fever, urinary tract infection, vibrio infections, yaws, yersinia infections, Yersinia pestis plague, zoonoses and zygomycosis.

The compounds presented herein can be effectively used against bacterial strains which have developed or are prone to or capable of developing resistance to at least one antimicrobial agents. Non-limiting examples of such bacterial strains include:
(a) Gram-positive bacteria such as Strep. pyogenes (Group A), Strep. pneumoniae, Strep. GpB, Strep. viridans, Strep. GpD-(Enterococcus), Strep. GpC and GpG, Staph. aureus, Staph. epidermidis, Bacillus subtilis, Bacillus anthraxis, Listeria monocytogenes, Anaerobic cocci, Clostridium spp., and Actinomyces spp; and
(b) Gram-negative bacteria such as Escherichia coli, Enterobacter aerogenes, Kiebsiella pneumoniae, Proteus mirabilis, Proteus vulgaris, Morganella morganii, Providencia stuartii, Serratia marcescens, Citrobacter freundii, Salmonella typhi, Salmonella paratyphi, Salmonella typhi murium, Salmonella virchow, Shigella spp., Yersinia enterocolitica, Acinetobacter calcoaceticus, Flavobacterium spp., Haemophilus influenzae, Pseudomonas aueroginosa, Campylobacter jejuni, Vibrio parahaemolyticus, Brucella spp., Neisseria meningitidis, Neisseria gonorrhoea, Bacteroides fragilis, and Fusobacterium spp.

According to some embodiments of the present invention, the compounds and/or complexes presented herein can be effectively used against bacterial strains which have developed or are prone to or capable of developing resistance to at least one antimicrobial agent.

According to some embodiments of the present invention, the compounds and/or complexes presented herein can be effectively used against bacterial strains which have developed or are prone to or capable of developing resistance to at least one antibacterial agent.

According to some embodiments of the present invention, the compounds and/or complexes presented herein can be effectively used against bacterial strains which have developed or are prone to or capable of developing resistance to a fluoroquinolone antibacterial agent.

Exemplary such bacterial strains include but not limited to, Escherichia coli strains such as E. coli R477-100, E. coli ATCC 25922, E. coli AG100B, E. coli ATCC 35218 and E. coli AG100A, B. subtilis strains (e.g., ATCC 6633), MRSA strains (e.g., ATCC 43300), and Pseudomonas aeruginosa strains.

According to an aspect of the present invention there is provided a method of treating a medical condition associated with a pathogenic microorganism in a subject in need thereof (e.g., a subject suspected as having, or diagnosed with, the medical condition). The method is effected by administering to the subject a therapeutically effective amount of a compound and/or a complex as described herein in any of the respective embodiments and any combination thereof (e.g., compounds of Formula I, Ia, Ib, Ic, IIa, IIb, IIc, IV, IVa, IVb, IVc, Va, Vb or Vc, and metal complexes thereof as described herein in any of the respective embodiments and any combination therein).

As used herein, the phrase “therapeutically effective amount” describes an amount of an active agent being administered, which will relieve to some extent one or more of the symptoms of the condition being treated. In the context of the present embodiments, the phrase “therapeutically effective amount” describes an amount of a compound being administered and/or re-administered, which will relieve to some extent one or more of the symptoms of the condition being treated by being at a level that is harmful to the target microorganism(s), and cause a disruption to the life-cycle of the target microorganism(s), namely a bactericidal level or otherwise a level that inhibits the microorganism growth or eradicates the microorganism.

The efficacy of any antimicrobial agent, including the compounds presented herein, is oftentimes referred to in minimal inhibitory concentration units, or MIC units. A MIC is the lowest concentration of an antimicrobial agent, typically measured in micro-molar (μM) or micrograms per milliliter (g/ml) units, which can inhibit the growth of a microorganism after a period of incubation, typically 24 hours. MIC values are used as diagnostic criteria to evaluate resistance of microorganisms to an antimicrobial agent, and for monitoring the activity of an antimicrobial agent in question. MICs are determined by standard laboratory methods, as these are described and demonstrated in the Examples section that follows. Standard laboratory methods typically follow a standard guideline of a reference body such as the Clinical and Laboratory Standards Institute (CLSI), British Society for Antimicrobial Chemotherapy (BSAC) or The European Committee on Antimicrobial Susceptibility Testing (EUCAST). In clinical practice, the minimum inhibitory concentrations are used to determine the amount of antibiotic agent that the subject receives as well as the type of antibiotic agent to be used.

According to an aspect of embodiments of the present invention, each of the compounds and/or metal complexes as described herein in any of the respective embodiments and any combination thereof is for use in treating a medical condition associated with a pathogenic microorganism and/or in treating a subject suspected as having, or diagnosed with a medical condition associated with a pathogenic microorganism.

According to an aspect of embodiments of the present invention, there is provided a use of any of the compounds and/or complexes as described herein in any of the respective embodiments and any combination thereof (e.g., compounds of Formula I, Ia, Ib, Ic, IIa, IIb, IIc, IV, IVa, IVb, IVc, Va, Vb or Vc, and metal complexes thereof as described herein in any of the respective embodiments and any combination therein) as a medicament or in the manufacture of a medicament. In some embodiments, the medicament is for treating a medical condition associated with a pathogenic microorganism and/or a subject suspected as having, or diagnosed with, a medical condition associated with a pathogenic microorganism.

The compounds presented herein can be administered via any administration route, including, but not limited to, orally, by inhalation, or parenterally, for example, by intravenous drip or intraperitoneal, subcutaneous, intramuscular or intravenous injection, or topically (including ophtalmically, vaginally, rectally, intranasally).

According to some of any of the embodiments described herein, the compounds or metal complexes as described herein act in a catalytic manner, and are used in a catalytically effective amount, that is, for example, a catalytic amount sufficient to catalyze reduction in a load of the pathogenic microorganism in a subject.

According to some of any of the embodiments described herein, a compound or a metal complex as described herein is capable of cleaving a microbial (e.g., bacterial DNA) and/or of interfering with microbial (e.g., bacterial DNA gyrase activity). These activities can be determined, for example, as described in the Examples section that follows.

According to some of any of the embodiments described herein, a compound or a metal complex as described herein is capable of fragmenting microbial (e.g., bacterial) supercoiled plasmid DNA into linear DNA.

According to some of any of the embodiments described herein, a compound or a metal complex as described herein acts in a catalytic manner, such that, for example, each molecule of a compound or a metal complex can repeatedly cleave a microbial (e.g., bacterial) DNA and/or of interfere with microbial (e.g., bacterial) DNA gyrase activity and/or fragment microbial (e.g., bacterial) supercoiled plasmid DNA into linear DNA.

According to some of any of the embodiments described herein, the compounds and/or complexes are used in an amount at the micromolar or nanomolar range, that is, at a concentration lower than 1 mM, for example, from 0.1 micromolar to 900 micromolar.

Pharmaceutical Compositions:

In any of the methods and uses described herein, the compounds and/or complexes as described herein (e.g., compounds of Formula I, Ia, Ib, Ic, IIa, IIb, IIc, IV, IVa, IVb, IVc, Va, Vb or Vc, and metal complexes thereof as described herein in any of the respective embodiments and any combination therein) can be utilized either per se or form a part of a pharmaceutical composition, which further comprises a pharmaceutically acceptable carrier, as defined herein.

According to an aspect of some embodiments of the present invention, there is provided a pharmaceutical composition which comprises, as an active ingredient, any of the compounds and/or complexes as described herein in any of the respective embodiments and any combination thereof and a pharmaceutically acceptable carrier.

As used herein a “pharmaceutical composition” refers to a preparation of the compounds presented herein, with other chemical components such as pharmaceutically acceptable and suitable carriers and excipients. The purpose of a pharmaceutical composition is to facilitate administration of an active agent (e.g., a compound and/or complex as described herein in any of the respective embodiments and any combination thereof) to an organism.

Hereinafter, the term “pharmaceutically acceptable carrier” refers to a carrier or a diluent that does not cause significant irritation to an organism and does not abrogate the biological activity and properties of the administered active agent. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of an active agent. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, PA, latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the compounds presented herein into preparations which, can be used pharmaceutically. Proper formulation is dependent upon the route of administration chosen.

According to some embodiments, the administration is effected orally. For oral administration, the compounds and/or complexes as presented herein can be formulated readily by combining the compounds with pharmaceutically acceptable carriers well known in the art. Such carriers enable the compounds and/or complexes as presented herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the compounds and/or complexes as presented herein may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds and/or complexes as presented herein may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active agent doses.

For buccal administration, the compositions may take the form of tablets or lozenges formulated in conventional manner.

For administration by inhalation, the compounds and/or complexes as presented herein are conveniently delivered in the form of an aerosol spray presentation (which typically includes powdered, liquefied and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compounds presented herein and a suitable powder base such as, but not limited to, lactose or starch.

The compounds and/or complexes as presented herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the compounds and/or complexes as preparation in water-soluble form. Additionally, suspensions of the compounds and/or complexes as presented herein may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds presented herein to allow for the preparation of highly concentrated solutions.

Alternatively, the compounds and/or complexes as presented herein may be in powder form for constitution with a suitable vehicle, e.g., sterile, pyrogen-free water, before use.

The compounds and/or complexes as presented herein may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of compounds and/or complexes as presented herein effective to prevent, alleviate or ameliorate symptoms of the disorder, or prolong the survival of the subject being treated.

Determination of a therapeutically effective amount is well within the capability of those skilled in the art, especially in light of the detailed disclosure provided herein.

For any compounds and/or complexes as presented herein used in the methods of the present embodiments, the therapeutically effective amount or dose can be estimated initially from activity assays in animals. For example, a dose can be formulated in animal models to achieve a circulating concentration range that includes the mutation suppression levels as determined by activity assays (e.g., the concentration of the test compounds and/or complexes which achieves a substantial read-through of the truncation mutation). Such information can be used to more accurately determine useful doses in humans.

Toxicity and therapeutic efficacy of the compounds and/or complexes as presented herein can be determined by standard pharmaceutical procedures in experimental animals, e.g., by determining the EC₅₀ (the concentration of a compound and/or complexes as where 50% of its maximal effect is observed) and the LD₅₀ (lethal dose causing death in 50% of the tested animals) for a subject active agent. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Dosage amount and interval may be adjusted individually to provide plasma levels of the compounds and/or complexes as presented herein which are sufficient to maintain the desired effects, termed the minimal effective concentration (MEC). The MEC will vary for each preparation, but can be estimated from in vitro data; e.g., the concentration of the compounds and/or complexes as necessary to achieve 50-90% expression of the whole gene having a truncation mutation, i.e. read-through of the mutation codon. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the chronic condition to be treated, dosing can also be a single periodic administration of a slow release composition described hereinabove, with course of periodic treatment lasting from several days to several weeks or until sufficient amelioration is effected during the periodic treatment or substantial diminution of the disorder state is achieved for the periodic treatment.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient(s). The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a compound and/or complex according to the present embodiments, formulated in a compatible pharmaceutical carrier may also be prepared, placed in an appropriate container, and labeled for treatment of an indicated condition or diagnosis, as is detailed hereinabove.

Thus, in some embodiments, the pharmaceutical composition is packaged in a packaging material and identified in print, in or on the packaging material, for use in the treatment of a medical condition associated with a pathogenic microorganism, as defined herein in any of the respective embodiments.

In any of the composition, methods and uses described herein, the compounds and/or complexes can be utilized in combination with other agents useful in the treatment of the medical conditions described herein.

According to some of any of the embodiments described herein, when the pharmaceutical composition comprises a metal complex as described herein in any of the respective embodiments, the composition may further comprise one or more agents that may protect the metal ion from inactivation by endogeneous components, as described herein.

Similarly, in any of the methods and uses described herein, which employ a metal complex as described herein in any of the respective embodiments and any combination thereof, the metal complex or the pharmaceutical composition comprising same can be used (e.g., administered) in combination with one or more agents that may protect the metal ion from inactivation by endogeneous components, as described herein.

According to some of these embodiments, the agents that may protect the metal ion include a redox reactive agent, such as, but not limited to, DTT, as described herein.

It is expected that during the life of a patent maturing from this application additional relevant fluoroquinolone and other antibiotic skeletons will be developed and the scope of the term fluoroquinolone or quinolone derivatives is intended to include all such new technologies a priori.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Herein throughout, the phrase “linking moiety” or “linking group” describes a group that connects two or more moieties or groups in a compound. A linking moiety is typically derived from a bi- or tri-functional compound, and can be regarded as a bi- or tri-radical moiety, which is connected to two or three other moieties, via two or three atoms thereof, respectively.

Exemplary linking moieties include a hydrocarbon moiety or chain, optionally interrupted by one or more heteroatoms, as defined herein, and/or any of the chemical groups listed below, when defined as linking groups.

When a chemical group is referred to herein as “end group” it is to be interpreted as a substituent, which is connected to another group via one atom thereof.

Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, in any order, each can be substituted or unsubstituted, and can be interrupted by one or more heteroatoms. The number of carbon atoms can range from 1 to 20, and is preferably lower, e.g., from 1 to 10, or from 1 to 6, or from 1 to 4. A hydrocarbon can be a linking group or an end group. An aliphatic or non-aromatic hydrocarbon does not include an aryl or heteroaryl group.

As used herein, the term “amine” describes both a —NR′R″ group and a —NR′— group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

Further alternatively, R′ and R″ form together a heteroalicyclic nitrogen-containing ring.

Herein throughout, an “amine-containing group” describes a chemical group that comprises or consists of at least one —NR′— or —NR′R″ group, with R′ and R″ is each independently hydrogen, alkyl, or cycloalkyl, or R′ and R″ form together a heterocyclic (e.g., alicyclic) group, or as defined hereinafter.

An amine-containing group can alternatively be a chemical group that comprises one or more —NR′— or —NR′R″ group(s) as defined herein, as part of a larger group that comprises additional chemical groups. Examples of such groups include, without limitation, amide, thioamide, carbamate, thiocarbamate, or polyamine-containing groups such as, but not limited to, guanyl, guanidyl, hydrazine, hydrazide, thiohydrazide, urea, and thiourea. In some embodiments, a polyamine-containing group is or comprises a guanidyl (guanidine).

Preferably, one or more amine groups in an amine or polyamine-containing group is such that has pKa around physiological pH (6-8, or about 7) or below it, such that it is not fully protonated in physiological environment, as in the case of, for example, guanidine or amine.

The term “alkyl” describes a saturated aliphatic hydrocarbon including straight chain and branched chain groups. Preferably, the alkyl group has 1 to 30, or 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. The alkyl group may be substituted or unsubstituted.

The alkyl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

Alkene and Alkyne, as used herein, are an alkyl, as defined herein, which contains one or more double bond or triple bond, respectively.

The term “cycloalkyl” describes an all-carbon monocyclic ring or fused rings (i.e., rings which share an adjacent pair of carbon atoms) group where one or more of the rings does not have a completely conjugated pi-electron system. Examples include, without limitation, cyclohexane, adamantine, norbornyl, isobornyl, and the like. The cycloalkyl group may be substituted or unsubstituted.

The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino, oxalidine, and the like.

The heteroalicyclic may be substituted or unsubstituted. The heteroalicyclic group can be an end group, as this phrase is defined hereinabove, where it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

A “guanidino” or “guanidine” or “guanidinyl” or “guanidyl” group refers to an —RaNC(═NRd)-NRbRc group, where each of Ra, Rb, Re and Rd can each be as defined herein for R′ and R″.

A “guanyl” or “guanine” group refers to an RaRbNC(═NRd)- group, where Ra, Rb and Rd are each as defined herein for R′ and R″.

In some of any of the embodiments described herein, the guanidine group is —NH—C(═NH)—NH₂.

In some of any of the embodiments described herein, the guanyl group is H₂N—C(═NH)-group.

Any one of the amine (including modified amine), guanidine and guanine groups described herein is presented as a free base form thereof, but is meant to encompass an ionized form thereof at physiological pH, and/or within a salt thereof, e.g., a pharmaceutically acceptable salt thereof, as described herein.

Whenever an alkyl, cycloalkyl, aryl, alkaryl, heteroaryl, heteroalicyclic, acyl and any other moiety as described herein is substituted, it includes one or more substituents, each can independently be, but are not limited to, hydroxy, alkoxy, thiohydroxy, thioalkoxy, aryloxy, thioaryloxy, alkaryl, alkenyl, alkynyl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide, as these terms are defined herein, unless otherwise indicated.

The terms “halide” or “halo” or “halogen” are used interchangeably and describe fluorine, chlorine, bromine or iodine.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking group, as these phrases are defined hereinabove, where R′ is as defined hereinabove.

The term “thiosulfate” describes a —O—S(═S)(═O)—OR′ end group or a —O—S(═S)(═O)—O— linking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)—OR′ end group or an —S(═O)—O— group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” describes a —S(═O)₂—R′ end group or an —S(═O)₂— linking group, as these phrases are defined hereinabove, where R′ is as defined herein.

The term “S-sulfonamide” describes a —S(═O)₂—NR′R″ end group or a —S(═O)₂—NR′-linking group, as these phrases are defined hereinabove, with R′ and R″ as defined herein.

The term “N-sulfonamide” describes an R'S(═O)₂—NR″— end group or a —S(═O)₂—NR′-linking group, as these phrases are defined hereinabove, where R′ and R″ are as defined herein.

The term “disulfide” refers to a —S—SR′ end group or a —S—S— linking group, as these phrases are defined hereinabove, where R′ is as defined herein.

The term “phosphonate” describes a —P(═O)(OR′)(OR″) end group or a —P(═O)(OR′)(O)-linking group, as these phrases are defined hereinabove, with R′ and R″ as defined herein.

The term “thiophosphonate” describes a —P(═S)(OR′)(OR″) end group or a —P(═S)(OR′)(O)— linking group, as these phrases are defined hereinabove, with R′ and R″ as defined herein.

The term “phosphinyl” describes a —PR′R″ end group or a —PR′— linking group, as these phrases are defined hereinabove, with R′ and R″ as defined hereinabove.

The term “phosphine oxide” describes a —P(═O)(R′)(R″) end group or a —P(═O)(R′)— linking group, as these phrases are defined hereinabove, with R′ and R″ as defined herein.

The term “phosphine sulfide” describes a —P(═S)(R′)(R″) end group or a —P(═S)(R′)— linking group, as these phrases are defined hereinabove, with R′ and R″ as defined herein.

The term “phosphite” describes an —O—PR′(═O)(OR″) end group or an —O—PH(═O)(O)-linking group, as these phrases are defined hereinabove, with R′ and R″ as defined herein.

The term “carbonyl” or “carbonate” as used herein, describes a —C(═O)—R′ end group or a —C(═O)— linking group, as these phrases are defined hereinabove, with R′ as defined herein.

The term “thiocarbonyl” as used herein, describes a —C(═S)—R′ end group or a —C(═S)-linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N—OH end group or a ═N—O— linking group, as these phrases are defined hereinabove.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The “hydroxyalkyl” is also referred to herein as “alcohol”, and describes an alkyl, as defined herein, substituted by a hydroxy group.

The term “cyano” describes a —C≡N group.

The term “isocyanate” describes an —N═C═O group.

The term “isothiocyanate” describes an —N═C═S group.

The term “nitro” describes an —NO₂ group.

The term “acyl halide” describes a —(C═O)R″″ group wherein R″″ is halide, as defined hereinabove.

The term “azo” or “diazo” describes an —N═NR′ end group or an —N═N— linking group, as these phrases are defined hereinabove, with R′ as defined hereinabove.

The term “peroxo” describes an —O—OR′ end group or an —O—O— linking group, as these phrases are defined hereinabove, with R′ as defined hereinabove.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O— thiocarboxylate.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone.

Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′— end group or a —OC(═O)—NR′-linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate.

The term “thiocarbamate” as used herein encompasses N-thiocarbamate and 0-thiocarbamate.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′-linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′-linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″— end group or a R′C(═O)—N— linking group, as these phrases are defined hereinabove, where R′ and R″ are as defined herein.

An amide can be linear or cyclic. When cyclic, R′ and the carbon atom are linked together to form a ring, in C-amide, and this group is also referred to as lactam. Cyclic amides can function as a linking group, for example, when an atom in the formed ring is linked to another group.

The term “guanyl” also describes a R′R″NC(═N)— end group or a —R′NC(═N)— linking group, as these phrases are defined hereinabove, where R′ and R″ are as defined herein.

The term “guanidine” also describes a —R′NC(═N)—NR″R′″ end group or a —R′NC(═N)— NR″— linking group, as these phrases are defined hereinabove, where R′, R″ and R′″ are as defined herein.

The term “hydrazine” describes a —NR′—NR″R′″ end group or a —NR′—NR″— linking group, as these phrases are defined hereinabove, with R′, R″, and R′″ as defined herein.

As used herein, the term “hydrazide” describes a —C(═O)—NR′—NR″R′″ end group or a —C(═O)—NR′—NR″— linking group, as these phrases are defined hereinabove, where R′, R″ and R′″ are as defined herein.

As used herein, the term “thiohydrazide” describes a —C(═S)—NR′—NR″R′″ end group or a —C(═S)—NR′—NR″— linking group, as these phrases are defined hereinabove, where R′, R″ and R′″ are as defined herein.

Herein throughout, the term “acyl” describes a —C(═O)—R group, wherein R is as described herein.

Herein throughout, the term “acyl” describes a —C(═O)—R group, with R being a substituted or unsubstituted alkyl, cycloalkyl, aryl, alkaryl, a hydrocarbon chain, or hydrogen.

According to some of any of the embodiments described herein, any of the compounds prepared or provided according to the present embodiments can be in a form of a pharmaceutically acceptable salt thereof.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, and/or to improve its stability, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., an amine-containing group such as amine and/or guanidyl and/or guanyl) group of the compound which is in a positively charged form (e.g., wherein the basic group is protonated), in combination with at least one counter-ion, derived from the selected base, that forms a pharmaceutically acceptable salt.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

The present embodiments further encompass any enantiomers, diastereomers, prodrugs, solvates, hydrates and/or pharmaceutically acceptable salts of the compounds described herein.

As used herein, the term “enantiomer” refers to a stereoisomer of a compound that is superposable with respect to its counterpart only by a complete inversion/reflection (mirror image) of each other. Enantiomers have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

General:

¹H-NMR and ¹³C-NMR, DEPT, COSY, HMQC and HMBC spectra were recorded on a Bruker Avance™ 600/500/400/300 spectrometers. Chemical shifts reported (in ppm) are relative to CHCl₃ (δ=7.26 ppm) with CDCl₃ as the solvent, to HOD or internal MeOH (δ=4.79 ppm) with D₂O as the solvent and to CD₃OH (δ=3.31 ppm) with MeOH as the solvent.

Mass spectral (MS) analyses were performed on a Bruker Maxis Impact under electron spray ionization (ESI+) QTOF MS, or on a Thermo LCQ fleet under electron spray ionization (ESI+).

Reactions were monitored by thin layer chromatography (TLC) on Silica gel 60 F254 (0.25 mm, Merck), and spots were visualized by UV lamp, iodine or charring with a yellow solution containing (NH₄)₆Mo₇O₂₄·4H₂O (120 grams) and (NH₄)₂Ce(NO₃)₆ (5 grams) in 10% H₂SO₄ (800 mL).

Flash column chromatography was performed on Silica gel Gel 60 (70-230 mesh).

EPR spectra were recorded on a Bruker EMX-10/12 X-band (v=9.3 GHz) digital EPR spectrometer. The spectra were recorded at a microwave power of 200 mW, 100 kHz magnetic field modulation of 3G amplitude. Digital field resolution was 2048 points per spectrum. Spectra processing and simulation were performed with the Bruker WIN-EPR and SimFonia Software.

UV-VIS spectra were recorded on an Ultrospec 2100 pro spectrometer.

Analytical HPLC was performed on Thermo Dionex UltiMate 3000, by using Phenomenex® C18 column and a detection wavelength of 271 nm.

Potentiometric titration was performed in accordance to Encyclopedia of Analytical Science, 2005, Pages 114-121.

All chemicals unless otherwise stated, were obtained from commercial sources. Purity of the final conjugates 1-6 and their Cu(II) salts were determined using HPLC analysis which indicated at least 95% purity unless otherwise stated.

Removal of Ligands Prior to Electrophoresis:

Removal of Ligands Prior to Electrophoresis was performed when more than 100 μM of Cu(II)-ligand or Co(III) ligand (at any concentration) complexes were employed that is, for DNA cleavage assays that were performed in the absence of any adjuvants. An Amberlyst® 15 (Sigma Aldrich) ion-exchange resin was prepared according to the reported literature [Hettich et al. J. Am. Chem. Soc. 1997, 119 (24), 5638-5647] and stored at −20° C. Before use, 0.5 gram of the exchanger was mixed with 250 μL of water. After stopping the DNA-cleavage reaction by addition of EDTA (54 mM, 15 minutes incubation at 37° C.), 10 μL of the exchanger suspension was added, vortexed, and incubated at 37° C. for 15 minutes before centrifugation and electrophoresis.

DNA Cleavage Assays:

DNA cleavage activity of the tested Complexes towards supercoiled (+) pHOT-1 plasmid DNA (TopoGEN) was monitored by gel electrophoresis. In a typical experiment, plasmid DNA (200 ng, 0.007 μg·mL⁻¹) in HEPES buffer (50 mM, pH 7.4) was mixed with different concentrations of a tested metal complex in the presence or absence of ascorbic acid (0.32 mM). All stock solutions of buffers and of the metal complexes were prepared using HPLC grade water (ChromAR®). Molecular biology reagent grade water (Sigma) was added up to a total reaction volume of 30 μL before incubation for a given time. For the ascorbic acid assays, the reaction was quenched immediately after incubation with EDTA (54 mM). For analysis, 10 μL of loading buffer (40% sucrose, 100 mM Tris-HCl (pH 7.5), 1 mM EDTA, 0.5 mg·mL⁻¹ bromophenol blue) was added to the incubated solution and loaded onto an agarose (SeaKem LE) gel [1% in TAE×1 buffer (40 mM Tris, 20 mM acetic acid, 1 mM EDTA)]. Electrophoresis was carried out at 60 V for 2 hours. Gels were stained with 1 μg·mL⁻¹ ethidium bromide for 30 minutes and de-stained in TAE×1 buffer. DNA bands were visualized with medium-range ultraviolet light using Bio-Rad Gel Doc XR+ imaging system. The quantity of different DNA forms (I or II) was estimated using ImageJ software.

E. coli Gyrase-DNA Supercoiling Assay:

The DNA supercoiling reactions were based on the manufacturer protocol (TopoGEN). The amount of DNA gyrase used in each assay was optimized by testing various dilutions of the stock. The amount sufficient to supercoil 250 nanograms (ng) of the substrate in 1 hour at 37° C. was then used for testing the compounds (1.1 U as defined by manufacturer). Assays (30 μL) contained 250 nanograms of relaxed pHOT1 plasmid DNA in Tris-HCl buffer (35 mM), pH 7.5, containing KCl (24 mM), MgCl₂ (4 mM), DTT (2 mM), Spermidine (1.8 mM), ATP (1 mM), 6.5% (w/v) glycerol and bovine serum albumin (0.1 mg/ml). Reactions were incubated at 37° C. for 60 minutes. Reaction mixtures were stopped by the addition of 30 μL of loading buffer (40% sucrose, 100 mM Tris·HCl (pH 7.5), 1 mM EDTA, 0.5 mg×mL⁻¹ bromophenol blue) and worked up using 30 μL of 24:1 chloroform/isoamyl alcohol mixture; the aqueous layer was then analyzed using electrophoresis.

Electrophoresis, gel staining, DNA visualization and DNA quantification were performed as described for the DNA cleavage assays. The IC₅₀ values were defined as the drug concentration that reduced the enzymatic activity observed with drug-free controls by 50% using nonlinear regression, three parameter curve fit using GraFit 5 software.

E. coli Gyrase-DNA Cleavage Assay:

E. coli gyrase enzyme was purchased from TopoGEN and the DNA cleavage reactions were based on the protocol of Inspiralis. Assays (30 μL) contained supercoiled (+) pHOT1 plasmid (200 nanograms) and DNA gyrase (5 U as defined by manufacturer) in 35 mM Tris-HCl (pH 7.5), 24 mM KCl, 4 mM MgCl₂, 2 mM DTT, 1.8 mM Spermidine, 6.5% (w/v) glycerol and 0.1 mg/mL bovine serum albumin. Reactions were incubated at 37° C. for 60 minutes. Enzyme-DNA cleavage complexes were trapped by adding 3 μL of 2% SDS. Following this, 1.5 μL of 10 mg/mL Proteinase K (Sigma-Aldrich) was added (where relevant) and the reaction mixtures were incubated at 37° C. for 30 minutes to digest the enzyme gyrase. Samples were mixed with 30 μL of loading buffer (40% sucrose, 100 mM Tris-HCl (pH 7.5), 1 mM EDTA, 0.5 mg-ml-1 bromophenol blue) and worked up using 30 μL of 24:1 chloroform/isoamyl alcohol mixture; the aqueous layer was then analyzed using electrophoresis. Electrophoresis, gel staining, DNA visualization and DNA quantification were performed as described for the DNA cleavage assays. DNA double stranded cleavage was monitored by the conversion of supercoiled plasmid to linear DNA and quantified in comparison to a control drug-free reaction.

Antibacterial Activity:

Comparative antibacterial activities were determined by measuring the MIC values by using the double-microdilution method according to the National Committee for Clinical Laboratory Standards (NCCLS). The Luria-Bertani growth medium and polypropylene 96-well plates (Thermo) were used unless otherwise stated. All the experiments were performed in triplicate, and analogous results were obtained in three different experiments.

### Example 1

### Design and Syntheses

Design Rationale:

It has been documented that fluoroquinolones selectively target prokaryotic topoisomerase IIA enzymes and therefore significantly discriminate between the pathogen and human cells [Aldred, K. J. et al. ACS Chem. Biol. 8, 2660-2668 (2013)]. Recent X-ray structures of moxifloxacin bound to the topo IV-DNA complex, and ciprofloxacin bound to the Gyrase-DNA complex, revealed that fluoroquinolones bind in a very similar fashion to both targets [Bax, B. D. et al. Nature 466, 935-940 (2010)]. That is, the two drug molecules intercalate in the gap between the −1 and +1 nucleotides (relative to the break induced by the enzyme), at the two ends of the 4-bp staggered cut (see, for example, FIG. 12A).

In both target complexes, the bulky C7 substituent of each drug molecule is stacked in very close proximity to the DNA backbone (between the +4 and +5 positions) of the adjacent DNA strand.

Several SAR studies on fluoroquinolones have demonstrated a high tolerance for structural variations at the C7 position of the fluoroquinolone (see, FIG. 5A). This phenomenon can be rationalized by the structural information available; the piperazine ring is seen to project into a large, solvent-accessible volume of space above the cleaved DNA (see, FIG. 2).

In order for a drug to generate a catalytic cycle, the chemical modification at the target must not only deactivate the target but also interfere with the affinity of the binding moiety to the target, as implied by the mechanism shown FIG. 1. Catalytic DNA cleavage at the site of the quinolone-topoisomerase-DNA ternary complex has the potential to satisfy both of these requirements: Once the DNA-topoisomerase IIA complex breaks apart, double nicked DNA would be released from the topoisomerase IIA enzyme, thus effectively fragmenting the chromosome and releasing the ciprofloxacin-nuclease compounds for another cycle.

Efficient DNase activity of quinolone-nuclease conjugates within the ternary complex would provide significant enhancement in their antimicrobial activity, and may offer a new catalytic mode of action that has the potential to slow down the development of resistance.

The following three key aspects were considered: (i) The choice of ‘catalytic warhead’ that efficiently cleaves the DNA substrate, (ii) the choice of attachment site of a ‘catalytic warhead’ on a fluoroquinolone (e.g. ciprofloxacin) scaffold, and (iii) the linker structure that connects the e.g., ciprofloxacin to the ‘catalytic warhead’.

The choice of ‘catalytic warhead’: The following delineates some background knowledge in this regard. Phosphodiester bonds are extremely stable towards hydrolysis: the half-life for hydrolysis of phosphodiester bonds in DNA at neutral pH and 25° C. is estimated to be 16 million years [Wolfenden et al. Chem. Rev. 2006, 106 (8), 3379-3396]. Despite such unique stability, natural nuclease enzymes cleave DNA very efficiently, providing impressive rate acceleration [Cleland et al. Chem. Rev. 2006, 106 (8), 3252-3278]. For most natural metallic nucleases, magnesium is the metal of choice, while in a few other cases divalent Zn or Ca are used [Dupureur, C. M. Curr. Opin. Chem. Biol. 2008, 12 (2), 250-255]. In this regard, numerous studies have reported on the design of strongly Lewis acidic metal complexes that promote hydrolysis of the DNA phosphodiester backbone [Yu, Z. and Cowan, J. Curr. Opin. Chem. Biol. 2018, 43, 37-42]. Although very efficient, metal-independent natural nucleases exist, artificial non-metallic hydrolytic DNA cleavage remains a challenge and the rate enhancements of reported compounds are generally not as good as those of metal-containing systems.

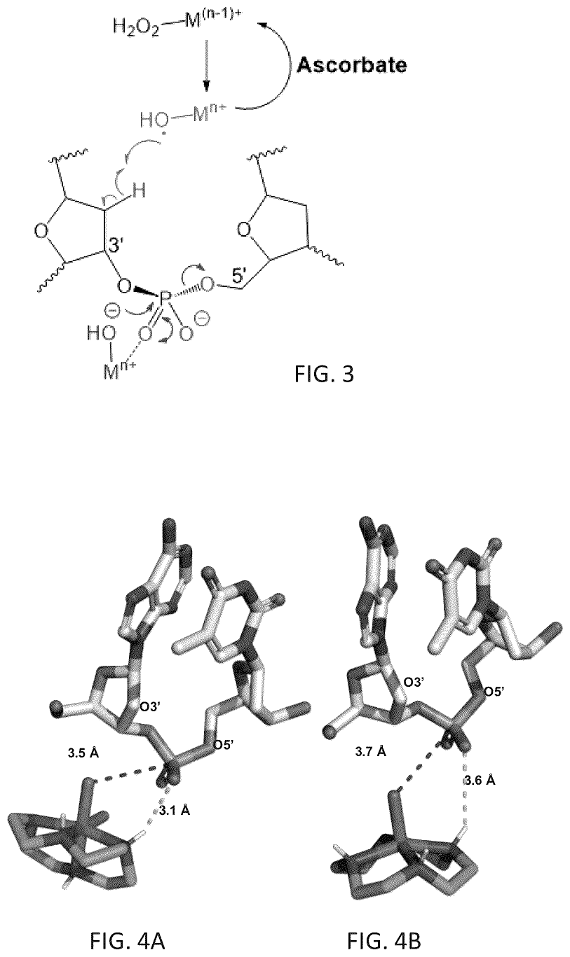

An alternative approach is to use redox active metals that efficiently cleave DNA in the presence of an oxidant (H₂O₂ or O₂) and endogenous reductant (e.g. ascorbic acid), by generating ROS such as the hydroxyl radical, which typically abstract hydrogen from the deoxyribose ring followed by spontaneous cleavage of C—C and C—O bonds [Yu and Cowan, 2018, supra], as shown in FIG. 3.

In both these approaches, it is essential that the metal-ligand complex is highly stable, since both prokaryotic and eukaryotic cells have sophisticated acquisition systems to scavenge essential metals from their environment, as an imbalance in metal homeostasis is deleterious [Veyrier, F. J. and Cellier, M. F. Front. Cell. Infect. Microbiol. 2015, 4 (190)].

A 1,4,7,10-tetraazacyclododecane (cyclen) was selected as an exemplary scaffold and Cu(II)-cyclen and Co(III)-cyclen as exemplary nuclease warheads. Cu(II)-cyclen is thermodynamically stable, and cleaves DNA primarily by metal bound-ROS in the presence of redox adjuvants [Joyner et al. J. Am. Chem. Soc. 2011, 133 (39), 15613-15626]. Although Cu(II)-cyclen itself has poor nuclease activity in the absence of adjuvants [Hettich, R. and Schneider, H.-J. J. Am. Chem. Soc. 1997, 119 (24), 5638-5647], it has been assumed that the Cu(II)-cyclen warhead, when connected with a spacer to a fluoroquinolone such as ciprofloxacin, should show enhanced hydrolytic and/or oxidative activity (compared to Cu(II)-cyclen alone) due to the proximity effect or its increased effective molarity [Page, M. I. and Jencks, W. P. Proc. Natl. Acad. Sci. 1971, 68 (8), 1678-1683]. Co(III)-cyclen is even more thermodynamically stable in view of the exchange-inertness of Co(III), and is more active than the Cu(II)-cyclen in the absence of adjuvants, via a hydrolytic pathway [Hettich, R. and Schneider, H.-J. J. Am. Chem. Soc. 1997, 119 (24), 5638-5647].

(ii) The choice of attachment site of a ‘catalytic warhead’ and (iii) the linker structure: The following delineates some background knowledge in this regard. Previous studies on SAR of fluoroquinolones have demonstrated a high tolerance for structural variations at the 7-position of the phenyl ring [Hubschwerlen et al. Bioorg. Med. Chem. 2003, 11 (10), 2313-2319; Kerns et al. Bioorg. Med. Chem. Lett. 2003, 13 (13), 2109-2112] (see, FIG. 5A). This has been attributed to the bulky substituent at the 7-position which projects into a large, solvent-accessible volume of space above the cleaved DNA.

Ciprofloxacin was modified at the terminal nitrogen of the piperazine moiety, while assuming that the catalytic warhead should be joined to the piperazine component via a ‘hydrophobic linker’ to avoid off-target polar interactions between the linker and the surrounding amino acid residues.

The length and flexibility (e.g., aliphatic vs. aromatic) of the linker was tested in order to explore a wide array of attack trajectories toward the scissile DNA phosphodiester bond, since in order to facilitate hydrolytic DNA cleavage one of the cyclen N—H^{δ+} bonds needs to be suitably positioned to provide the necessary phosphate stabilization as a hydrogen bond donor whilst the metal-activated nucleophile (water) must be well orientated for an in-line nucleophilic attack, as shown in FIGS. 4A-B. These design principles were then used together with molecular docking (Autodock 4.2 and Ledock; data not shown) to design Compounds 1-6, as presented in FIG. 5A. As shown therein, in compounds 1-3 the cyclen warhead is connected to ciprofloxacin via an aliphatic spacer, and in compounds 4-6 via an aromatic spacer. The docking data (data not shown) for all the Cu(II) and Co(III) complexes of Compounds 1-6 predict that (i) the ciprofloxacin scaffold will bind in the ‘native’ pocket and that (ii) the cyclen warhead will be brought into very close proximity to the DNA.

Synthesis of Compounds 1-6 and their Cu(II) and Co(III) Complexes:

Compounds 1-6 were synthesized in five or six chemical steps, as detailed in FIG. 5B. First, commercially available ciprofloxacin was ester-protected under acidic conditions to give methyl ester 7. Compound 7 was then treated with an excess of the relevant di-bromo compound under basic conditions to afford compounds 8a-f containing a terminal bromide. While the majority of the dibromo compounds used here are commercially available, the dibromo derivatives 1-(2-bromoethyl)-4-(bromomethyl) benzene (10) and 1-(bromomethyl)-4-(3-bromopropyl) benzene (11), required for preparing 8e and 8f are not commercially available. Therefore, 10 and 11 were synthesized from commercially available starting materials in two and four steps respectively as shown in FIG. 5C and further detailed hereinbelow.

Initially, the bromide 8a was treated with tri-Boc-cyclen under basic conditions in an attempt at preparing pure 9a in one step. However, the reaction did not proceed even after being heated at high temperature for a long time. These conditions were only successful for the preparation of 9d. For the rest of the compounds, instead of using tri-Boc-cyclen, the bromo derivative was first treated with free cyclen under base conditions followed by Boc-protection of the remaining secondary amines, to yield the corresponding tri-Boc protected derivatives. For example, compound 8a was first coupled to free cyclen under basic conditions (Cs₂CO₃, CH₃CN), then Boc-protected and finally 9a was isolated using column chromatography. Thus, 9b-c and 9e-f were synthesized via cyclen as described for 9a.

Finally, compounds 9a-f were treated sequentially with strong base (LiOH) and then strong acid (TFA) to remove the ester and Boc protecting groups. Compounds 1-6 were then converted to the free amine form using an AmberLite™ ion-exchange column. The final 1-6 structures were characterized by NMR and MS techniques.

The ligands 1-6 were treated with aqueous Cu(II) chloride to afford the corresponding mono-aqua Cu(II) complexes. The complexes were characterized by UV-VIS, EPR and HMRS. The aqua-hydroxo Co(III) complexes of 1-6 were prepared in three steps according to a previously described procedure [Jorge et al. Chem.—A Eur. J. 2016, 22 (11), 3764-3774]. The initial Co(III)-CO₃ complexes were characterized by MS, UV-VIS and ¹³C-NMR, the intermediate Co(III)-Cl₂ complexes were characterized by UV-VIS and HRMS and the aqua-hydroxo-Co(III) complexes were characterized by UV-VIS.

Preparation of Compound 7: Dowex 50 WX8 ion-exchange resin (H⁺ form, mesh 200-400, 27 grams) was set stirring with commercially available ciprofloxacin (10 grams, 0.03 mol) in MeOH (500 mL) and refluxed under an argon atmosphere. TLC analysis [a mixture of MeOH:DCM:MeNH₂ (33% in ethanol) in a ratio 10:10:0.5]) indicated almost complete consumption of the starting material after 48 hours. The crude reaction mixture was neutralized with 2.5% NH₄OH in methanol at 0° C., transferred to a sintered glass column and then washed extensively with 2.5% NH₄OH in methanol until all of the product had been eluted. The solvent was then evaporated to yield a white solid (8.4 grams, 24.3 mmol, 81%).

¹H NMR (300 MHz, CDCl₃): δ_{H}=8.52 (s, 1H, Quinolone H-2), 8.02 (d, J_{HF}=13.4 Hz, 1H, QH-5), 7.25 (d, J_{HF}=7.1 Hz, 1H, QH-8), 3.90 (s, 3H, OCH₃), 3.47-3.37 (m, 1H, cyclopropane CH), 3.27-3.18 (m, 4H, piperazine 2×CH₂), 3.13-3.04 (m, 4H, piperazine 2×CH₂), 1.35-1.26 (m, 2H, cyclopropane CH₂), 1.16-1.08 (m, 2H, cyclopropane CH₂);

¹³C NMR (126 MHz, CDCl₃): δ_{C}=173.02 (d, J_{CF}=2.2 Hz, C═O), 166.18 (s, C═O), 152.69 (d, J_{CF}=248.8 Hz, Ar), 148.31 (s, C—H Ar), 144.28 (d, J_{CF}=10.8 Hz, Ar), 137.96 (d, J_{CF}=0.9 Hz, Ar), 122.05 (d, J_{CF}=7.6 Hz, Ar), 112.32 (d, J_{CF}=21.0 Hz, C—H Ar), 109.72 (s, C—H Ar), 104.19 (d, J_{CF}=3.3 Hz, C—H Ar), 51.98 (s, OCH₃), 50.35 (d, J_{CF}=4.4 Hz, piperazine CH₂), 45.91 (s, piperazine CH₂), 34.59 (s, cyclopropane CH), 8.12 (s, cyclopropane CH₂).

ESI+ QTOFMS calculated for C₁₈H₂₁FN₃O₃ ([M+H]⁺) m/e 346.15; measured m/e 346.10.

Preparation of Compound 10a: 4-(Hydroxymethyl)phenylacetic acid (3.5 grams, 21.1 mmol) was added to dry THF (100 mL) and set stirring under an argon atmosphere but with a vent. The reaction mixture was then cooled to 0° C. and then BH₃·SMe₂ (2M in THF, 37 mL, 73.7 mmol) was added at which point the solution began to effervesce; the vent and ice-bath were both maintained for the duration of the reaction. TLC analysis (ethyl acetate) indicated complete consumption of the starting material after 3 hours. The reaction mixture was quenched with 1M HCl(aq) and then worked up with ethyl acetate and water. The organic layers were combined, dried with MgSO₄ and then evaporated to yield 10a as a colorless oil (3.007 grams, 19.8 mmol, 94%).

¹H NMR (300 MHz, CD₃OD): δ_{H}=7.27 (d, J=8.2 Hz, 2H, Ar), 7.20 (d, J=8.2 Hz, 2H, Ar), 4.56 (s, 2H, Ph-CH₂OH), 3.73 (t, J=7.1 Hz, 2H, CH₂—CH₂OH), 2.81 (t, J=7.1 Hz, 2H, CH₂CH₂OH);

ESI+ QTOFMS calculated for C₉H₁₁O ([M-OH]⁺) m/e 135.08; measured m/e 135.08.

Preparation of Compound 10: The complex was synthesized according to a previously reported procedure [Pechlivanidis et al. European J. Org. Chem. 2009, 2009 (2), 223-237]. Yield 59% (1.58 gram, 5.68 mmol);

¹H NMR (300 MHz, CDCl₃): δ_{H}=7.36 (d, J=8.1 Hz, 2H, Ar), 7.20 (d, J=8.1 Hz, 2H, Ar), 4.49 (s, 2H, Ph-CH₂Br), 3.56 (t, J=7.6 Hz, 2H, CH₂—CH₂Br), 3.17 (t, J=7.6 Hz, 2H, CH₂CHBr); ESI+ QTOFMS calculated for C₉H₁₀Br ([M-Br]⁺) m/e 197.00; measured m/e 197.00.

Preparation of Compound 11a: The compound was synthesized according to a previously reported (general) procedure for Sonogashira coupling of propargyl alcohol to benzyl halide derivatives [Cheng et al. Org. Biomol. Chem. 2016, 14 (16), 3878-3882]. Yield 84% (2.72 g, 16.8 mmol);

¹H NMR (300 MHz, CD₃OD): δ_{H}=7.42-7.27 (m, 2H, Ar), 7.34-7.29 (m, 2H, Ar), 4.60 (s, 2H, CH₂OH), 4.39 (s, 2H, CH₂OH);

13C NMR (126 MHz, CD₃OD): δ_{C}=143.14 (Ar), 132.54 (Ar), 127.85 (Ar), 123.04 (Ar), 88.58 (alkyne carbon), 85.39 (alkyne carbon), 64.68 (CH₂OH), 51.17 (CH₂OH).

Preparation of Compound 11b: To a solution of 11a (1.7 gram, 10.5 mmol) in dry DMF (35 mL) was added TBDMSCl (4.1 gram, 27.3 mmol) and imidazole (3.57 gram, 52.5 mmol) and then set stirring under argon at room temperature. TLC analysis (Hex:EA 29:1) indicated complete consumption of 11a after 16 hours. Workup was performed using hexane and brine. The organic layers were dried with MgSO₄, filtered and then evaporated to yield 11b (3.84 grams, 9.83 mmol, 94%);

¹H NMR (200 MHz, CDCl₃): δ_{H}=7.44-7.35 (m, 2H, Ar), 7.30-7.21 (m, 2H, Ar), 4.73 (s, 2H, CH₂OTBDMS), 4.54 (s, 2H, CH₂OTBDMS), 0.94 (bs, 18H, 2×Si—C(CH₃)₃), 0.18-0.15 (s, 6H, 2×Si—CH₃), 0.10-0.18 (s, 6H, 2×Si—CH₃).

Preparation of Compound 11c: To an ethyl acetate solution (80 mL) of 11b (4.29 grams, 11 mmol) was added Pd/C 10% (820 mg, 0.77 mmol). The reaction beaker (three-necked) was then de-gassed and subsequently flushed with argon three times. The reaction beaker was then washed with an H₂(g) balloon (1 atm.) and then two fresh H₂(g) balloons were set in place. TLC analysis (Hex:EA 18:2 and Hex:EA 14.5:0.25) indicated complete consumption of the starting material after 16 hours. The crude mixture was filtered through Celite and then washed extensively with EA. The solvent was then evaporated to yield 11c (3.82 grams, 9.68 mmol, 88%);

¹H NMR (300 MHz, CDCl₃): δ_{H}=7.41 (d, J=8.0 Hz, 2H, Ar), 7.33 (d, J=8.0 Hz, 2H, Ar), 4.89 (s, 2H, Ph-CH₂—OTBDMS), 3.80 (t, J=6.5 Hz, 2H, Ph-(CH₂)₂—CH₂—OTBDMS), 2.84 (t, J=7.8 Hz, 2H, Ph-CH₂—(CH₂)₂—OTBDMS), 2.06-1.93 (m, 2H, Ph-CH₂—CH₂—CH₂—OTBDMS), 1.12 (s, 9H, Si—C(CH₃)₃), 1.09 (s, 9H, Si—C(CH₃)₃), 0.27 (s, 6H, 2×Si—CH₃), 0.23 (s, 6H, 2×Si-CH₃).

Preparation of Compound 11: The complex was synthesized according to a previously reported procedure [Pechlivanidis et al. European J. Org. Chem. 2009, 2009 (2), 223-237] but with some small changes. Briefly, to a solution of 11c (3.82 grams, 9.7 mmol) in acetic acid (17 mL) was added 33% HBr/acetic acid (11 mL, 60.6 mmol) and then set stirring in a thick-walled ampule at 100° C. TLC analysis (Hex:EA 29:0.5) indicated complete consumption of the starting material after 4 hours. The reaction mixture was quenched by slow addition to NaHCO₃(aq) and ice, and then worked up with EA. The EA layers were then combined, dried with MgSO₄ and then evaporated to yield 11 as a colorless oil (2.017 grams, 6.9 mmol, 71%);

¹H NMR (300 MHz, CDCl₃): δ_{H}=7.33 (d, J=8.1 Hz, 2H, Ar), 7.18 (d, J=8.1 Hz, 2H, Ar), 4.49 (s, 2H, Ph-CH₂—Br), 3.39 (t, J=6.5 Hz, 2H, Ph-(CH₂)₂—CH₂—Br), 2.78 (t, J=7.4 Hz, 2H, Ph-CH₂—(CH₂)₂—Br), 2.22-2.09 (m, 2H, Ph-CH₂—CH₂—CH₂—Br);

¹³C NMR (126 MHz, CDCl₃): δ_{C}=140.89 (Ar), 135.58 (Ar), 129.15 (Ar), 128.93 (Ar), 33.85, 33.56, 33.49, 32.95.

ESI+ QTOFMS calculated for C₁₀H₁₁Br₂ ([M+H]⁺) m/e 290.9; measured m/e 290.7.

General Procedure for the Synthesis of 8a-d:

To a stirring suspension of 7 and N,N-diisopropylethylamine (DIPEA) (4 equivalents) in acetonitrile (35 mL/gram of 7) was added the appropriate dibromo compound (10 equivalents) under an atmosphere of argon and set stirring at 60° C. overnight. TLC analysis (DCM:MeOH 9:1) indicated complete consumption of 7 after 20 hours. The solvent was evaporated and the residue dried under vacuum overnight. The crude product was then loaded onto a DCM-packed silica column as a DCM solution; the desired product was eluted in 3% MeOH/DCM to yield 8a-d as white solids.

Compound 8a. Yield 54% (1.97 gram, 3.87 mmol);

¹H NMR (500 MHz, CDCl₃): δ_{H}=8.51 (s, 1H, QH-2), 7.99 (d, J_{HF}=13.4 Hz, 1H, QH-5), 7.25 (d, J_{HF}=7.2 Hz, 1H, QH-8), 3.89 (s, 3H, OCH₃), 3.45-3.39 (m, 1H, cyclopropane CH), 3.41 (t, J=7.0 Hz, 2H, linker CH₂Br), 3.28 (t, J=4.6 Hz, 4H, piperazine 2×CH₂), 2.66 (t, J=4.6 Hz, 4H, piperazine 2×CH₂), 2.41 (t, J=7.6 Hz, 2H, linker CH₂-piperazine), 1.87 (quint, J=7.0 Hz, 2H, linker CH₂), 1.54 (quint, J=7.5 Hz, 2H, linker CH₂), 1.47 (quint, J=7.5 Hz, 2H, linker CH₂), 1.36 (quint, J=7.0 Hz, 2H, linker CH₂), 1.33-1.27 (m, 2H, cyclopropane CH₂), 1.14-1.09 (m, 2H, cyclopropane CH₂);

¹³C NMR (126 MHz, CDCl₃): δ_{C}=173.13 (d, J_{CF}=2.2 Hz, C═O), 166.42 (s, C═O), 153.46 (d, J_{CF}=248.7 Hz, Ar), 148.40 (s, C—H Ar), 144.70 (d, J_{CF}=10.7 Hz, Ar), 138.05 (d, J_{CF}=1.1 Hz, Ar), 122.92 (d, J_{CF}=7.4 Hz, Ar), 113.18 (d, J_{CF}=23.0 Hz, C—H Ar), 109.96 (s, Ar), 104.86 (d, J_{CF}=3.5 Hz, C—H Ar), 58.51 (s, linker CH₂-piperazine), 53.06 (s, piperazine CH₂), 52.10 (s, OCH₃), 50.02 (d, J_{CF}=4.5 Hz, piperazine CH₂), 34.60 (s, cyclopropane CH), 33.40 (s, linker CH₂Br), 32.75 (s, linker CH₂), 28.13 (s, linker CH₂), 26.74 (s, linker CH₂), 26.73 (s, linker CH₂), 8.19 (s, cyclopropane CH₂).

ESI+ QTOFMS calculated for C₂₄H₃₁BrFN₃O₃ ([M+H]⁺) m/e 508.15; measured m/e 508.15.

Compound 8b. Yield 61% (921 mg, 1.76 mmol);

¹H NMR (500 MHz, CDCl₃): δ_{H}=8.53 (s, 1H, QH-2), 8.03 (d, J_{HF}=13.3 Hz, 1H, QH-5), 7.26 (d, J_{HF}=7.1 Hz, 1H, QH-8), 3.91 (s, 3H, OCH₃), 3.45-3.39 (m, 1H, cyclopropane CH), 3.41 (t, J=6.8 Hz, 2H, linker CH₂Br), 3.31 (t, J=4.3 Hz, 4H, piperazine 2×CH₂), 2.73-2.66 (m, 4H, piperazine 2×CH₂), 2.45 (t, J=7.6 Hz, 2H, linker CH₂-piperazine), 1.86 (quint, J=7.0 Hz, 2H, linker CH₂), 1.60-1.51 (m, 2H, linker CH₂), 1.45 (quint, J=7.0 Hz, 2H, linker CH₂), 1.38-1.33 (m, 4H, 2×linker CH₂), 1.33-1.28 (m, 2H, cyclopropane CH₂), 1.15-1.10 (m, 2H, cyclopropane CH₂);

¹³C NMR (126 MHz, CDCl₃): δ_{C}=173.24 (s, C═O), 166.68 (s, C═O), 153.59 (d, J_{CF}=249.0 Hz, Ar), 148.51 (s, C—H Ar), 144.71 (d, J_{CF}=10.4 Hz, Ar), 138.16 (s, Ar), 123.23 (d, J_{CF}=6.7 Hz, Ar), 113.47 (d, J_{CF}=23.2 Hz, C—H Ar), 110.24 (s, Ar), 104.90 (m, C—H Ar), 58.65 (s, linker CH₂-piperazine), 53.07 (s, piperazine CH₂), 52.23 (s, OCH₃), 49.94 (m, piperazine CH₂), 34.64 (s, cyclopropane CH), 34.10 (s, linker CH₂Br), 32.83 (s, linker CH₂), 28.77 (s, linker CH₂), 28.19 (s, linker CH₂), 27.41 (s, linker CH₂), 26.69 (s, linker CH₂), 8.29 (s, cyclopropane CH₂).

ESI+ QTOFMS calculated for C₂₅H₃₄BrFN₃O₃ ([M+H]⁺) m/e 522.17; measured m/e 522.18.

Compound 8c. Yield 58% (1.8 gram, 3.36 mmol);

¹H NMR (500 MHz, CDCl₃): δ_{H}=8.43 (s, 1H, QH-2), 7.87 (d, J_{HF}=13.2 Hz, 1H, QH-5), 7.20 (d, J_{HF}=7.0 Hz, 1H, QH-8), 3.84 (s, 3H, OCH₃), 3.43-3.39 (m, 1H, cyclopropane CH), 3.37 (t, J=6.8 Hz, 2H, linker CH₂Br), 3.29-3.19 (m, 4H, piperazine 2×CH₂), 2.68-2.59 (m, 4H, piperazine 2×CH₂), 2.39 (t, J=7.4 Hz, 2H, linker CH₂-piperazine), 1.81 (quint, J=7.2 Hz, 2H, linker CH₂CH₂Br), 1.54-1.46 (m, 2H, linker CH₂CH₂-piperazine), 1.44-1.36 (m, 2H, linker CH₂), 1.34-1.24 (m, 8H, 3×linker CH₂, cyclopropane CH₂), 1.12-1.05 (m, 2H, cyclopropane CH₂);

¹³C NMR (126 MHz, CDCl₃): δ_{C}=173.08 (d, J_{CF}=2.1 Hz, C═O), 166.17 (s, C═O), 153.37 (d, J_{CF}=248.9 Hz, Ar), 148.31 (s, C—H Ar), 144.60 (d, J_{CF}=10.6 Hz, Ar), 137.99 (d, J_{CF}=1.0 Hz, Ar), 122.74 (d, J_{CF}=7.1 Hz, Ar), 113.00 (d, J_{CF}=23.2 Hz, C—H Ar), 109.75 (s, Ar), 104.88 (d, J_{CF}=3.0 Hz, C—H Ar), 58.62 (s, linker CH₂-piperazine), 52.97 (s, piperazine CH₂), 51.98 (s, OCH₃), 49.87 (d, J_{CF}=4.5 Hz, piperazine CH₂), 34.61 (s, cyclopropane CH), 34.09 (s, linker CH₂Br), 32.78 (s, linker CH₂CH₂Br), 29.33 (s, linker CH₂), 28.68 (s, linker CH₂), 28.09 (s, linker CH₂), 27.39 (s, linker CH₂), 26.73 (s, linker CH₂CH₂-piperazine), 8.14 (s, cyclopropane CH₂).

ESI+ QTOFMS calculated for C₂₆H₃₆BrFN₃O₃ ([M+H]⁺) m/e 536.19; measured m/e 536.11.

Compound 8d. Yield 22% (828 mg, 1.57 mmol);

¹H NMR (500 MHz, CDCl₃): δ_{H}=8.52 (s, 1H, QH-2), 8.01 (d, J_{HF}=13.3 Hz, 1H, QH-5), 7.38-7.37 (d, J=8.2 Hz, 2H, linker Ar), 7.34 (d, J=8.2 Hz, 2H, linker Ar), 7.25 (d, J_{HF}=7.0 Hz, 1H, QH-8), 4.50 (s, 2H, CH₂Br), 3.90 (s, 3H, OCH₃), 3.58 (s, 2H, linker CH₂-piperazine), 3.43-3.37 (m, 1H, cyclopropane CH), 3.27 (t, J=4.7 Hz, 4H, piperazine 2×CH₂), 2.66 (t, J=4.7 Hz, 4H, piperazine 2×CH₂), 1.32-1.27 (m, 2H, cyclopropane CH₂), 1.14-1.10 (m, 2H, cyclopropane CH₂);

¹³C NMR (126 MHz, CDCl₃): δ_{C}=173.24 (s, C═O), 166.66 (s, C═O), 153.58 (d, J_{CF}=248.9 Hz, Ar), 148.49 (s, C—H Ar), 144.82 (d, J_{CF}=10.8 Hz, Ar), 138.39 (s, linker Ar), 138.15 (s, Ar), 136.95 (s, linker Ar), 129.70 (s, C—H linker Ar), 129.20 (s, C—H linker Ar), 123.13 (d, J_{CF}=7.2 Hz, Ar), 113.44 (d, J_{CF}=23.3 Hz, C—H Ar), 110.20 (s, Ar), 104.86 (m, C—H Ar), 62.66 (s, linker CH₂-piperazine), 52.95 (s, piperazine CH₂), 52.22 (s, OCH₃), 50.11 (d, J_{CF}=3.9 Hz, piperazine CH₂), 34.63 (s, cyclopropane CH), 33.52 (s, linker CH₂Br), 8.26 (s, cyclopropane CH₂).

ESI+ QTOFMS calculated for C₂₆H₂₈BrFN₃O₃ ([M+H]⁺) m/e 528.12; measured m/e 528.02.

General Procedure for the Synthesis of 8e-f:

A suspension of 7 and DIPEA (4 eq.) in CH₃CN (35 mL/gram of 7) was set stirring in an ice bath and then the appropriate dibromo compound (10 or 11) was added (1.2 eq.) under an atmosphere of argon. The ice-bath was maintained for the duration of the reaction. TLC analysis (DCM:MeOH 9:1) indicated complete consumption of 7 after 12 hours. The solvent was evaporated and the residue dried under vacuum overnight. The crude product was then loaded onto a DCM-packed silica column as a DCM solution; the desired product was eluted in 3% MeOH/DCM to yield 8e-f as white solids.

Compound 8e. Yield 60% (1.78 gram, 3.28 mmol);

¹H NMR (500 MHz, CDCl₃): δ_{H}=8.48 (s, 1H, QH-2), 7.95 (d, J_{HF}=13.4 Hz, 1H, QH-5), 7.30 (d, J=8.1 Hz, 2H, linker Ar), 7.22 (d, J_{HF}=7.1 Hz, 1H, QH-8), 7.18 (d, J=8.1 Hz, 2H, linker Ar), 3.88 (s, 3H, OCH₃), 3.56 (m, 4H, linker CH₂Br, linker CH₂-piperazine), 3.40 (m, 1H, cyclopropane CH), 3.26 (t, J=4.8 Hz, 4H, piperazine 2×CH₂), 3.15 (t, J=7.6 Hz, linker CH₂CH₂Br), 2.65 (t, J=4.6 Hz, 4H, piperazine 2×CH₂), 1.30-1.25 (m, 2H, cyclopropane CH₂), 1.13-1.09 (m, 2H, cyclopropane CH₂);

¹³C NMR (126 MHz, CDCl₃): δ_{C}=173.10 (s, C═O), 166.48 (s, C═O), 153.48 (d, J_{CF}=248.4 Hz, Ar), 148.40 (s, C—H Ar), 144.74 (d, J_{CF}=10.7 Hz, Ar), 138.05 (s, Ar), 137.96 (s, linker Ar), 136.54 (s, linker Ar), 129.53 (s, C—H linker Ar), 128.73 (s, C—H linker Ar), 122.94 (d, J_{CF}=6.9 Hz, Ar), 113.21 (d, J_{CF}=23.3 Hz, C—H Ar), 110.01 (s, Ar), 104.86 (m, C—H Ar), 62.72 (s, linker CH₂-piperazine), 52.89 (s, piperazine CH₂), 52.12 (s, OCH₃), 50.06 (d, J_{CF}=3.9 Hz, piperazine CH₂), 39.12 (s, linker CH₂CH₂Br), 34.59 (s, cyclopropane CH), 33.10 (s, linker CH₂Br), 8.19 (s, cyclopropane CH₂).

ESI+ QTOFMS calculated for C₂₇H₃₀BrFN₃O₃ ([M+H]⁺) m/e 542.14; measured m/e 542.15.

Compound 8f. Yield 63% (2.036 gram, 3.66 mmol);

¹H NMR (500 MHz, CDCl₃): δ_{H}=8.50 (s, 1H, QH-2), 7.97 (d, J_{HF}=13.4 Hz, 1H, QH-5), 7.27 (d, J=7.9 Hz, 2H, linker Ar), 7.23 (d, J_{HF}=7.1 Hz, 1H, QH-8), 7.16 (d, J=7.9 Hz, 2H, linker Ar), 3.89 (s, 3H, OCH₃), 3.56 (s, 2H, linker CH₂-piperazine), 3.35-3.44 (m, 3H, linker CH₂Br, cyclopropane CH), 3.26 (t, J=4.5 Hz, 4H, piperazine 2×CH₂), 2.76 (t, J=7.4 Hz, 2H, linker PhCH₂(CH₂)₂Br), 2.65 (t, J=4.5 Hz, 4H, piperazine 2×CH₂), 2.16 (tt, J=7.4 Hz, 6.8 Hz, 2H, linker PhCH₂CH₂CH₂Br) 1.31-1.26 (m, 2H, cyclopropane CH₂), 1.13-1.09 (m, 2H, cyclopropane CH₂);

¹³C NMR (126 MHz, CDCl₃): δ_{C}=173.16 (s, C═O), 166.55 (s, C═O), 153.51 (d, J_{CF}=251.1 Hz, Ar), 148.44 (s, C—H Ar), 144.78 (d, J_{CF}=10.8 Hz, Ar), 139.66 (s, linker Ar), 138.07 (s, Ar), 135.62 (s, linker Ar), 129.52 (s, C—H linker Ar), 128.63 (C—H linker Ar), 122.97 (d, J_{CF}=6.2 Hz, Ar), 113.25 (d, J_{CF}=23.1 Hz, C—H Ar), 110.03 (s, Ar), 104.86 (d, J_{CF}=2.5 Hz, C—H Ar), 62.77 (s, linker CH₂-piperazine), 52.88 (s, piperazine CH₂), 52.16 (s, OCH₃), 50.05 (d, J_{CF}=3.7 Hz, piperazine CH₂), 34.60 (s, cyclopropane CH), 34.24 (s, linker PhCH₂CH₂CH₂Br), 33.72 (s, linker PhCH₂(CH₂)₂Br), 33.24 (s, linker CH₂Br), 8.22 (s, cyclopropane CH₂).

ESI+ QTOFMS calculated for C₂₈H₃₁BrFN₃NaO₃ ([M+Na]⁺) m/e 578.14; measured m/e 578.13.

General Procedure for the Synthesis of 9a-c and 9e-f:

Step 1: The appropriate bromo derivative (8a-c, 8e-f), free-amine form of cyclen (2 mol equivalents) and Cs₂CO₃ (2.2 mol equivalents) were added to dry CH₃CN (35 mL/gram of bromo derivative) to form a suspension and set stirring under an argon atmosphere. The reaction mixture was then heated to 60° C. TLC analysis (DCM:MeOH:25% NH₄OH(aq) 15:3:0.3) indicated complete consumption of the bromo derivative after 20 hours (the unwanted elimination product of 8e appears at the same Rf as the starting material). The reaction mixture was filtered, the residue washed extensively with CH₃CN and then the filtrate evaporated under vacuum.

Step 2: The crude from the previous step and Et₃N (10 mol equivalents) were dissolved in dry DCM (35 mL/gram of bromo derivative) and set stirring in an ice bath under an argon atmosphere. Boc₂O (10 mol equivalents) was then added. The reaction mixture was allowed to rise to room temperature. TLC analysis (DCM:MeOH 9:1) indicated complete consumption of the starting material (the N-alkylated intermediate from step 1) after 15 hours. The solvent was evaporated and the residue dried under vacuum overnight. The crude product was then loaded onto a DCM-packed silica column as a DCM solution; the desired product was eluted in a solvent mixture of MeOH and DCM to yield 9a-c and 9e-f as white solids.

Compound 9a. Following the general procedure, compound 8a (880 mg, 1.73 mmol) yielded 45% over 2 steps (700 mg, 0.78 mmol);

¹H NMR (500 MHz, CDCl₃): δ_{H}=8.50 (s, 1H, QH-2), 7.98 (d, J_{HF}=13.4 Hz, 1H, QH-5), 7.24 (d, J_{HF}=7.0 Hz, 1H, QH-8), 3.88 (s, 3H, OCH₃), 3.57-3.45 (m, 4H, cyclen 2×CH₂), 3.44-3.35 (m, 3H, cyclopropane CH, cyclen CH₂), 3.34-3.16 (m, 10H, piperazine 2×CH₂, cyclen 3×CH₂), 2.70-2.54 (m, 8H, piperazine 2×CH₂, cyclen 2×CH₂), 2.49 (t, J=7.6 Hz, 2H, linker CH₂-cyclen), 2.40 (t, J=7.8 Hz, 2H, linker CH₂-piperazine), 2.1 (bs, 1H, acidic NH), 1.52 (quint, J=7.6 Hz, 2H, linker CH₂), 1.44 (s, 9H, Boc), 1.43 (s, 18H, 2×Boc), 1.37-1.21 (m, 8H, cyclopropane CH₂, linker 3×CH₂), 1.13-1.08 (m, 2H, cyclopropane CH₂);

¹³C NMR (126 MHz, CDCl3): δ_{C}=173.18 (s, C═O), 166.56 (s, C═O), 156.22 (s, Boc C═O), 155.80 (s, Boc C═O), 155.50 (s, Boc C═O), 153.52 (d, J_{CF}=248.9 Hz, Ar), 148.44 (s, C—H Ar), 144.74 (d, J_{CF}=10.8 Hz, Ar), 138.10 (s, Ar), 123.04 (d, J_{CF}=6.8 Hz, Ar), 113.33 (d, J_{CF}=23.2 Hz, C—H Ar), 110.08 (s, Ar), 104.84 (s, C—H Ar), 79.64 (s, Boc quaternary), 79.56 (s, Boc quaternary), 79.39 (s, Boc quaternary), 58.66 (s, linker CH₂-piperazine), 55.01 (m, cyclen CH₂ proximal to linker), 53.65 (m, cyclen CH₂ proximal to linker), 53.08 (s, piperazine CH₂), 52.58 (m, linker CH₂-cyclen), 52.15 (s, OCH₃), 50.03 (d, J_{CF}=3.6 Hz, piperazine CH₂), 48.49 (m, cyclen CH₂), 48.05 (m, cyclen CH₂), 47.67 (m, cyclen CH₂), 34.61 (s, cyclopropane CH), 28.79 (s, Boc CH₃), 28.63 (s, Boc CH₃), 28.58 (s, Boc CH₃), 27.90 (s, linker CH₂), 27.67 (s, linker CH₂), 26.97 (s, linker CH₂), 23.92 (s, linker CH₂), 8.22 (s, cyclopropane CH₂).

ESI+ QTOFMS: calculated for C₄₇H₇₅FN₇O₉ ([M+H]⁺) m/e 900.55; measured m/e 900.63.

Compound 9b. Following the general procedure, compound 8b (529 mg, 1.01 mmol) yielded 26% over 2 steps (242 mg, 0.26 mmol);

¹H NMR (500 MHz, CDCl₃): δ_{H}=8.50 (s, 1H, QH-2), 7.97 (d, J_{HF}=13.3 Hz, 1H, QH-5), 7.24 (d, J_{HF}=7.0 Hz, 1H, QH-8), 3.88 (s, 3H, OCH₃), 3.57-3.45 (m, 4H, cyclen 2×CH₂), 3.44-3.34 (m, 3H, cyclopropane CH, cyclen CH₂), 3.31-3.18 (m, 10H, piperazine 2×CH₂, cyclen 3×CH₂), 2.67-2.53 (m, 8H, piperazine 2×CH₂, cyclen 2×CH₂), 2.48 (t, J=7.5 Hz, 2H, linker CH₂-cyclen), 2.39 (t, J=8.0 Hz, 2H, linker CH₂-piperazine), 2.16 (bs, 1H, acidic NH), 1.51 (quint, J=7.4 Hz, 2H, linker CH₂), 1.44 (s, 9H, Boc), 1.43-1.41 (m, 20H, 2×Boc, linker CH₂), 1.35-1.19 (m, 8H, cyclopropane CH₂, linker 3×CH₂), 1.13-1.08 (m, 2H, cyclopropane CH₂);

¹³C NMR (126 MHz, CDCl₃): δ_{C}=173.18 (s, C═O), 166.55 (s, C═O), 156.21 (s, Boc C═O), 155.79 (s, Boc C═O), 155.49 (s, Boc C═O), 153.51 (d, J_{CF}=249.0 Hz, Ar), 148.43 (s, C—H Ar), 144.75 (d, J_{CF}=10.7 Hz, Ar), 138.08 (s, Ar), 123.02 (d, J_{CF}=6.8 Hz, Ar), 113.28 (d, J_{CF}=22.9 Hz, C—H Ar), 110.05 (s, Ar), 104.83 (s, C—H Ar), 79.62 (s, Boc quaternary), 79.54 (s, Boc quaternary), 79.37 (s, Boc quaternary), 58.70 (s, linker CH₂-piperazine), 55.00 (m, cyclen CH₂ proximal to linker), 53.62 (m, cyclen CH₂ proximal to linker), 53.07 (s, piperazine CH₂), 52.61 (m, linker CH₂-cyclen), 52.15 (s, OCH₃), 50.04 (d, J_{CF}=3.5 Hz, piperazine CH₂), 48.50 (m, cyclen CH₂), 48.05 (m, cyclen CH₂), 47.69 (m, cyclen CH₂), 34.60 (s, cyclopropane CH), 29.69 (s, linker CH₂), 28.79 (s, Boc CH₃), 28.62 (s, Boc CH₃), 28.56 (s, Boc CH₃), 27.87 (s, linker CH₂), 27.60 (s, linker CH₂), 26.91 (s, linker CH₂), 23.89 (s, linker CH₂), 8.21 (s, cyclopropane CH₂).

ESI+ QTOFMS calculated for C₄₈H₇₇FN₇O₉ ([M+H]⁺) m/e 914.58; measured m/e 914.58.

Compound 9c. Following the general procedure, compound 8c (1.25 gram, 2.32 mmol) yielded 49% over 2 steps (1.06 gram, 1.14 mmol);

¹H NMR (500 MHz, CDCl₃): δ_{H}=8.40 (s, 1H, QH-2), 7.85 (d, J_{HF}=13.2 Hz, 1H, QH-5), 7.17 (d, J_{HF}=7.1 Hz, 1H, QH-8), 3.82 (s, 3H, OCH₃), 3.53-3.46 (m, 2H, cyclen CH₂), 3.46-3.41 (m, 2H, cyclen CH₂), 3.41-3.30 (m, 3H, cyclopropane CH, cyclen CH₂), 3.28-3.12 (m, 10H, cyclen 3×CH₂, piperazine 2×CH₂), 2.64-2.50 (m, 8H, cyclen 2×CH₂, piperazine 2×CH₂), 2.44 (t, J=7.9 Hz, 2H, linker CH₂-cyclen), 2.35 (t, J=7.6 Hz, 2H, linker CH₂-piperazine), 1.50-1.43 (m, 2H, linker CH₂CH₂-piperazine), 1.40 (bs, 9H, Boc), 1.39-1.37 (m, 20H, 2×Boc, linker CH₂CH₂-cyclen), 1.30-1.21 (m, 8H, linker 3×CH₂, cyclopropane CH₂), 1.21-1.14 (m, 2H, linker CH₂), 1.10-1.03 (m, 2H, cyclopropane CH₂);

¹³C NMR (126 MHz, CDCl₃): δ_{C}=172.95 (s, C═O), 166.19 (s, C═O), 156.08 (s, Boc C═O), 155.67 (s, Boc C═O), 155.39 (s, Boc C═O), 153.33 (d, J_{CF}=248.9 Hz, Ar), 148.25 (s, C—H Ar), 144.58 (d, J_{CF}=10.6 Hz, Ar), 137.93 (s, Ar), 122.72 (d, J_{CF}=6.7 Hz, Ar), 112.95 (d, J_{CF}=23.0 Hz, C—H Ar), 109.77 (s, Ar), 104.80 (d, J_{CF}=2.8 Hz, C—H Ar), 79.49 (s, Boc quaternary), 79.40 (s, Boc quaternary), 79.23 (s, Boc quaternary), 58.64 (s, linker CH₂-piperazine), 54.95 (m, cyclen CH₂ proximal to linker), 53.64 (m, cyclen CH₂ proximal to linker), 52.98 (s, piperazine CH₂), 52.61 (m, linker CH₂-cyclen), 51.92 (s, OCH₃), 49.91 (d, J_{CF}=3.5 Hz, piperazine CH₂), 48.42 (m, cyclen CH₂), 47.94 (m, cyclen CH₂), 47.59 (m, cyclen CH₂), 34.52 (s, cyclopropane CH), 29.57 (s, linker CH₂), 29.54 (s, linker CH₂), 28.68 (s, Boc CH₃), 28.52 (s, Boc CH₃), 28.46 (s, Boc CH₃), 27.77 (s, linker CH₂), 27.46 (s, linker CH₂), 26.82 (s, linker CH₂), 23.91 (m, linker CH₂CH₂-cyclen), 8.07 (s, cyclopropane CH₂).

ESI+ QTOFMS calculated for C₄₉H₇₉FN₇O₉ ([M+H]⁺) m/e 928.59; measured m/e 928.60.

Compound 9e. Following the general procedure, compound 8e (1.38 gram, 2.54 mmol) yielded 34% over 2 steps (802 mg, 0.86 mmol);

¹H NMR (500 MHz, CDCl₃): δ_{H}=8.49 (s, 1H, QH-2), 7.97 (d, J_{HF}=13.3 Hz, 1H, QH-5), 7.27 (d, J=7.6 Hz, 2H, linker Ar), 7.23 (d, J_{HF}=7.0 Hz, 1H, QH-8), 7.15 (d, J=7.7 Hz, 2H, linker Ar), 3.88 (s, 3H, OCH₃), 3.55 (bs, 2H, linker CH₂-piperazine), 3.54-3.48 (m, 2H, cyclen CH₂), 3.47-3.42 (m, 2H, cyclen CH₂), 3.42-3.22 (m, 13H, cyclopropane CH, cyclen 4×CH₂, piperazine 2×CH₂), 2.85-2.68 (m, 8H, cyclen 2×CH₂, linker CH₂-cyclen, linker CH₂CH₂-cyclen), 2.64 (t, J=4.5 Hz, 4H, piperazine 2×CH₂), 1.45-1.42 (m, 27H, 3×Boc), 1.30-1.25 (m, 2H, cyclopropane CH₂), 1.13-1.08 (m, 2H, cyclopropane CH₂);

¹³C NMR (126 MHz, CDCl₃): δ_{C}=173.17 (d, J_{CF}=2.2 Hz, C═O), 166.54 (s, C═O), 156.25 (s, Boc C═O), 155.83 (s, Boc C═O), 155.45 (s, Boc C═O), 153.50 (d, J_{CF}=248.7 Hz, Ar), 148.43 (s, C—H Ar), 144.77 (d, J_{CF}=11.0 Hz, Ar), 139.31 (bs, linker Ar), 138.07 (d, J_{CF}=1.0 Hz, Ar), 135.55 (bs, linker Ar), 129.55 (s, C—H linker Ar), 128.76 (s, C—H linker Ar), 122.98 (d, J_{CF}=6.9 Hz, Ar), 113.27 (d, J_{CF}=23.1 Hz, C—H Ar), 110.04 (s, Ar), 104.85 (d, J_{CF}=2.9 Hz, C—H Ar), 79.74 (s, Boc quaternary), 79.63 (s, Boc quaternary), 79.41 (s, Boc quaternary), 62.73 (s, linker CH₂-piperazine), 54.78 (m, cyclen CH₂ proximal to linker), 54.18 (m, linker CH₂-cyclen), 53.25 (m, cyclen CH₂ proximal to linker), 52.85 (s, piperazine CH₂), 52.14 (s, OCH₃), 50.04 (d, J_{CF}=4.5 Hz, piperazine CH₂), 48.40 (m, cyclen CH₂), 48.05 (m, cyclen CH₂), 47.68 (m, cyclen CH₂), 34.59 (s, cyclopropane CH), 29.77 (m, linker CH₂CH₂-cyclen), 28.79 (s, Boc CH₃), 28.64 (s, Boc CH₃), 28.56 (s, Boc CH₃), 8.21 (s, cyclopropane CH₂).

ESI+ QTOFMS calculated for C₅₀H₇₃FN₇O₉ ([M+H]⁺) m/e 934.54; measured m/e 934.56.

Compound 9f. Following the general procedure, compound 8f (1.41 gram, 2.53 mmol) yielded 55% over 2 steps (1.32 gram, 1.39 mmol);

¹H NMR (500 MHz, CDCl₃): δ_{H}=8.52 (s, 1H, QH-2), 8.00 (d, J_{HF}=13.3 Hz, 1H, QH-5), 7.28-7.22 (m, 3H, linker Ar, QH-8), 7.14 (d, J=8.0 Hz, 2H, linker Ar), 3.89 (s, 3H, OCH₃), 3.55 (bs, 2H, linker CH₂-piperazine), 3.55-3.49 (m, 2H, cyclen CH₂), 3.49-3.43 (m, 2H, cyclen CH₂), 3.43-3.31 (m, 3H, cyclopropane CH, cyclen CH₂), 3.31-3.22 (m, 8H, piperazine 2×CH₂, cyclen 2×CH₂), 3.22-3.14 (m, 2H, cyclen CH₂), 2.69-2.52 (m, 12H, cyclen 2×CH₂, piperazine 2×CH₂, linker CH₂-cyclen, linker CH₂(CH₂)₂-cyclen), 1.95 (bs, 1H, NH), 1.77 (m, 2H, linker CH₂CH₂-cyclen), 1.45 (bs, 9H, Boc), 1.43 (bs, 18H, 2×Boc), 1.31-1.26 (m, 2H, cyclopropane CH₂), 1.14-1.08 (m, 2H, cyclopropane CH₂);

¹³C NMR (126 MHz, CDCl₃): δ_{C}=173.20 (s, C═O), 166.65 (s, C═O), 156.20 (s, Boc C═O), 155.80 (s, Boc C═O), 155.48 (s, Boc C═O), 153.54 (d, J_{CF}=248.7 Hz, Ar), 148.46 (s, C—H Ar), 144.81 (d, J_{CF}=10.5 Hz, Ar), 141.02 (s, linker Ar), 138.10 (s, Ar), 135.35 (bs, linker Ar), 129.43 (s, C—H linker Ar), 128.36 (s, C—H linker Ar), 123.05 (d, J_{CF}=6.9 Hz, Ar), 113.35 (d, J_{CF}=23.3 Hz, C—H Ar), 110.11 (s, Ar), 104.85 (bs, C—H Ar), 79.68 (s, Boc quaternary), 79.58 (s, Boc quaternary), 79.40 (s, Boc quaternary), 62.78 (s, linker CH₂-piperazine), 55.05 (bs, cyclen CH₂ proximal to linker), 53.66 (bs, cyclen CH₂ proximal to linker), 52.87 (s, piperazine CH₂), 52.19 (s, OCH₃), 52.02 (bs, linker CH₂-cyclen), 50.07 (d, J_{CF}=3.5 Hz, piperazine CH₂), 48.42 (bs, cyclen CH₂), 48.06 (bs, cyclen CH₂), 47.68 (m, cyclen CH₂), 34.59 (s, cyclopropane CH), 33.71 (s, CH₂(CH₂)₂-cyclen), 28.79 (s, Boc CH₃), 28.63 (s, Boc CH₃), 28.57 (s, Boc CH₃), 25.87 (m, CH₂CH₂-cyclen), 8.23 (s, cyclopropane CH₂).

ESI+ QTOFMS calculated for C₅₁H₇₅FN₇O₉ ([M+H]⁺) m/e 948.56; measured m/e 948.64.

Preparation of Compound 9d: Compound 8d (410 mg, 0.78 mmol), tri-Boc-cyclen (660 mg, 1.40 mmol) and DIPEA (0.4 mL, 2.34 mmol) were dissolved in dry DCM (40 mL). The reaction mixture was set stirring at room temperature. TLC analysis (DCM:MeOH 15:1) indicated complete consumption of the bromo derivative after two weeks. The solvent was evaporated and then the residue dried under vacuum overnight. The crude product was loaded onto a DCM-packed silica column as a DCM solution; the desired product was eluted in DCM:MeOH gradient from 35:1 to 30:1, to yield 9d as a white solid (520 mg, 0.57 mmol, 72%).

¹H NMR (500 MHz, CDCl₃): δ_{H}=8.44 (s, 1H, QH-2), 7.89 (d, J_{HF}=13.2 Hz, 1H, QH-5), 7.25 (d, J=7.85 Hz, 2H, linker Ar), 7.20 (d, J_{HF}=7.0 Hz, 1H, QH-8), 7.17 (d, J=7.85 Hz, 2H, linker Ar), 3.83 (s, 3H, OCH₃), 3.68 (bs, 2H, linker CH₂-cyclen), 3.58-3.48 (m, 6H, cyclen 2×CH₂, linker CH₂-piperazine), 3.43-3.12 (m, 13H, piperazine 2×CH₂, cyclen 4×CH₂, cyclopropane CH), 2.70-2.42 (m, 8H, piperazine 2×CH₂, cyclen 2×CH₂), 1.41 (s, 9H, Boc), 1.38 (s, 18H, 2×Boc), 1.28-1.23 (m, 2H, cyclopropane CH₂), 1.09-1.03 (m, 2H, cyclopropane CH₂);

¹³C NMR (126 MHz, CDCl₃): δ_{C}=173.06 (d, J_{CF}=1.8 Hz, C═O), 166.24 (s, C═O), 156.07 (s, Boc C═O), 155.73 (s, Boc C═O), 155.29 (s, Boc C═O), 153.36 (d, J_{CF}=248.8 Hz, Ar), 148.30 (s, C—H Ar), 144.60 (d, J_{CF}=10.5 Hz, Ar), 137.98 (s, Ar), 136.54 (s, linker Ar), 135.82 (s, linker Ar), 130.33 (s, C—H linker Ar), 129.07 (s, C—H linker Ar), 122.78 (d, J_{CF}=7.1 Hz, Ar), 113.00 (d, J_{CF}=23.4 Hz, C—H Ar), 109.77 (s, Ar), 104.89 (d, J_{CF}=2.6 Hz, C—H Ar), 79.54 (s, Boc quaternary), 79.39 (s, Boc quaternary), 79.32 (s, Boc quaternary), 62.52 (s, linker CH₂-piperazine), 56.83 (m, linker CH₂-cyclen), 55.85 (m, cyclen CH₂ proximal to linker), 54.86 (m, cyclen CH₂ proximal to linker), 52.73 (s, piperazine CH₂), 51.96 (s, OCH₃), 49.81 (d, J_{CF}=3.4 Hz, piperazine CH₂), 48.21 (m, cyclen CH₂), 47.70 (m, cyclen CH₂), 47.34 (m, cyclen CH₂), 34.58 (s, cyclopropane CH), 28.68 (bs, Boc CH₃), 28.44 (bs, Boc CH₃), 8.11 (s, cyclopropane CH₂).

ESI+ QTOFMS calculated for C₄₉H₇₁FN₇O₉ ([M+H]⁺) m/e 920.53; measured m/e 920.53.

General Procedure for the Synthesis of Compounds 1-6:

Step 1: The appropriate derivative (9a-f) was dissolved in a mixture of MeOH and H₂O (5:1, 65 mL/gram of starting material). An aqueous solution of LiOH (5 eq.) was then added and the reaction mixture was stirred at room temperature. TLC analysis (15:1 DCM:MeOH and 25:1:1 DCM:MeOH:25% NH₄OH(aq)) indicated complete consumption of the starting material after 20 hours. The solvents were then evaporated, followed by workup with diethyl ether and brine. The desired product was isolated in the organic phase, which was dried with MgSO₄ and then evaporated to yield a white solid.

Step 2: The crude from the previous step was dissolved in a mixture of TFA and DCM (1:1, 30 mL/gram of derivative 9a-f) and set stirring at room temperature. TLC analysis (15:15:10:5 MeOH:DCM:MeNH₂ [33 wt. % in ethanol]:H₂O and 10:10:5 MeOH:DCM: 25% aqueous NH₄OH) indicated complete consumption of the starting material after 15 hours. The TFA and DCM were evaporated and then the residue dried under vacuum overnight. The dry crude was then dissolved in water, neutralized with aqueous NaHCO₃ and then loaded onto an AmberLite™ CG50 (H⁺-form, 100-200 mesh) column. The column was washed sequentially with H₂O, MeOH, MeOH:H₂O and then once again H₂O. The desired product was then eluted using a mixture of 2.5% NH₄OH_{(aq)}, MeOH and H₂O in a ratio of 1:3:6 respectively. The solvent was then evaporated to yield 1-6 as white solids.

Compound 1. Following the general procedure, compound 9a (542 mg, 0.6 mmol) yielded 48% over 2 steps (167 mg, 0.29 mmol);

¹H NMR (500 MHz, CD₃OD): δ_{H}=8.64 (s, 1H, QH-2), 7.94 (d, J_{HF}=13.6 Hz, 1H, QH-5), 7.49 (d, J_{HF}=7.3 Hz, 1H, QH-8), 3.67-3.60 (m, 1H, cyclopropane CH), 3.32-3.27 (m, 4H, piperazine 2×CH₂), 2.93-2.86 (m, 12H, cyclen 6×CH₂), 2.70 (t, J=5.2 Hz, 4H, cyclen 2×CH₂), 2.59-2.54 (m, 4H, piperazine 2×CH₂), 2.52 (t, J=7.4 Hz, 2H, linker CH₂-cyclen), 2.18 (t, J=8.1 Hz, 2H, linker CH₂-piperazine), 1.48 (quint, J=7.5 Hz, 2H, linker CH₂), 1.43-1.32 (m, 4H, cyclopropane CH₂, linker CH₂), 1.24 (quint, J=7.5 Hz, 2H, linker CH₂), 1.19-1.10 (m, 4H, cyclopropane CH₂, linker CH₂);

¹³C NMR (126 MHz, CD₃OD): δ_{C}=176.75 (s, C═O), 172.34 (s, C═O), 154.62 (d, J_{CF}=247.8 Hz, Ar), 148.61 (s, C—H Ar), 145.95 (d, J_{CF}=10.8 Hz, Ar), 140.23 (s, Ar), 123.19 (d, J_{CF}=6.6 Hz, Ar), 116.59 (s, Ar), 113.04 (d, J_{CF}=23.1 Hz, C—H Ar), 106.64 (s, C—H Ar), 59.77 (s, linker CH₂-piperazine), 55.19 (s, linker CH₂-cyclen), 54.12 (s, piperazine CH₂), 51.37 (s, cyclen CH₂), 50.70 (d, J_{CF}=3.7 Hz, piperazine CH₂), 46.63 (s, cyclen CH₂), 44.99 (s, cyclen CH₂), 44.75 (s, cyclen CH₂), 35.83 (s, cyclopropane CH), 28.80 (s, linker CH₂), 28.64 (s, linker CH₂), 28.17 (s, linker CH₂), 27.64 (s, linker CH₂), 8.54 (s, cyclopropane CH₂).

HRMS (ESI+ QTOFMS) calculated for C₃₁H₄₈FN₇O₃ ([M+H]⁺) m/e 586.3875; measured m/e 586.3900.

Compound 2. Following the general procedure, compound 9b (511 mg, 0.56 mmol) yielded 41% over 2 steps (140 mg, 0.23 mmol);

¹H NMR (500 MHz, CD₃OD): δ_{H}=8.60 (s, 1H, QH-2), 7.92 (d, J_{HF}=13.1 Hz, 1H, QH-5), 7.45 (d, J_{HF}=7.1 Hz, 1H, QH-8), 3.62-3.54 (m, 1H, cyclopropane CH), 3.35-3.28 (m, 4H, piperazine 2×CH₂), 2.82-2.76 (m, 4H, cyclen 2×CH₂), 2.73-2.67 (m, 8H, piperazine 2×CH₂, cyclen 2×CH₂), 2.66-2.61 (m, 4H, cyclen 2×CH₂), 2.61-2.56 (m, 4H, cyclen 2×CH₂), 2.49-2.40 (m, 4H, linker CH₂-piperazine, linker CH₂-cyclen), 1.60-1.54 (m, 2H, linker CH₂), 1.52-1.46 (m, 2H, linker CH₂), 1.39-1.31 (m, 8H, cyclopropane CH₂, linker 3×CH₂), 1.16-1.10 (m, 2H, cyclopropane CH₂);

¹³C NMR (126 MHz, CD₃OD): δ_{C}=176.95 (s, C═O), 172.66 (s, C═O), 154.52 (d, J_{CF}=246.9 Hz, Ar), 148.64 (s, C—H Ar), 145.73 (d, J_{CF}=11.1 Hz, Ar), 139.99 (s, Ar), 123.53 (s, Ar), 117.58 (s, Ar), 113.10 (d, J_{CF}=23.4 Hz, C—H Ar), 106.42 (s, C—H Ar), 59.77 (s, linker CH₂-piperazine), 55.36 (s, linker CH₂-cyclen), 54.14 (s, piperazine CH₂), 52.17 (s, cyclen CH₂), 50.75 (d, J_{CF}=4.3 Hz, piperazine CH₂), 47.16 (s, cyclen CH₂), 46.02 (s, cyclen CH₂), 45.20 (s, cyclen CH₂), 35.63 (s, cyclopropane CH), 30.61 (s, linker CH₂), 28.70 (s, linker CH₂), 28.63 (s, linker CH₂), 28.45 (s, linker CH₂), 27.52 (s, linker CH₂), 8.48 (s, cyclopropane CH₂).

HRMS (ESI+ QTOFMS) calculated for C₃₂H₅₁FN₇O₃ ([M+H]⁺) m/e 600.4032; measured m/e 600.4007.

Compound 3. Following the general procedure, compound 9c (1.06 gram, 1.14 mmol) yielded 61% over 2 steps (430 mg, 0.70 mmol);

¹H NMR (500 MHz, CD₃OD): δ_{H}=8.60 (s, 1H, QH-2), 7.91 (d, J_{HF}=12.9 Hz, 1H, QH-5), 7.49 (d, J_{HF}=7.2 Hz, 1H, QH-8), 3.67-3.58 (m, 1H, cyclopropane CH), 3.36-3.30 (m, 4H, piperazine 2×CH₂), 2.94-2.79 (m, 12H, cyclen 6×CH₂), 2.74-2.59 (m, 8H, piperazine 2×CH₂, cyclen 2×CH₂), 2.47 (t, J=7.2 Hz, 2H, linker CH₂-cyclen), 2.34 (t, J=7.9 Hz, 2H, linker CH₂-piperazine), 1.50-1.39 (m, 4H, linker 2×CH₂), 1.39-1.32 (m, 2H, cyclopropane CH₂), 1.23-1.10 (m, 10H, cyclopropane CH₂, linker 4×CH₂);

¹³C NMR (126 MHz, CD₃OD): δ_{C}=176.61 (s, C═O), 172.35 (s, C═O), 154.54 (d, J_{CF}=247.2 Hz, Ar), 148.41 (s, C—H Ar), 145.85 (d, J_{CF}=10.6 Hz, Ar), 140.12 (s, Ar), 123.29 (s, Ar), 117.06 (s, Ar), 113.03 (d, C—H Ar), 106.58 (s, J_{CF}=2.4 Hz, C—H Ar), 59.85 (s, linker CH₂-piperazine), 55.14 (s, linker CH₂-cyclen), 54.18 (s, piperazine CH₂), 51.34 (s, cyclen CH₂), 50.76 (d, J_{CF}=4.2 Hz, piperazine CH₂), 46.57 (s, cyclen CH₂), 45.01 (s, cyclen CH₂), 44.67 (s, cyclen CH₂), 35.74 (s, cyclopropane CH), 30.84 (s, linker CH₂), 30.83 (s, linker CH₂), 28.77 (s, linker CH₂), 28.61 (s, linker CH₂), 27.99 (s, linker CH₂), 27.62 (s, linker CH₂), 8.54 (s, cyclopropane CH₂).

HRMS (ESI+ QTOFMS) calculated for C₃₃H₅₃FN₇O₃ ([M+H]⁺) m/e 614.4188; measured m/e 614.4145.

Compound 4. Following the general procedure, compound 9d (697 mg, 0.76 mmol) yielded 50% over 2 steps (232 mg, 0.38 mmol);

NMR analysis is of TFA salt: ¹H NMR (500 MHz, D₂O): δ_{H}=8.67 (s, 1H, QH-2), 7.61 (d, J=7.9 Hz, 2H, linker Ar), 7.56 (d, J_{HF}=12.8 Hz, 1H, QH-5), 7.54-7.50 (m, 3H, QH-8, linker Ar), 4.51 (s, 2H, linker CH₂-piperazine), 4.01-3.89 (m, 4H, piperazine CH₂, linker CH₂-cyclen), 3.76-3.66 (m, 3H, piperazine CH₂, cyclopropane CH), 3.51-3.42 (m, 2H, piperazine CH₂), 3.39-3.13 (m, 10H, piperazine CH₂, cyclen 4×CH₂), 3.07-2.95 (m, 4H, cyclen 2×CH₂), 2.92 (t, J=5.1 Hz, 4H, cyclen 2×CH₂), 1.45-1.39 (m, 2H, cyclopropane CH₂), 1.23-1.17 (m, 2H, cyclopropane CH₂);

¹³C NMR (126 MHz, D₂O): δ_{C}=176.08 (s, C═O), 169.01 (s, C═O), 162.69 (s, C═O of TFA), 153.34 (d, J_{CF}=251.3 Hz, Ar), 148.39 (s, C—H Ar), 144.04 (d, J_{CF}=10.7 Hz, Ar), 138.95 (s, Ar), 137.08 (s, linker Ar), 131.69 (s, C—H linker Ar), 130.67 (s, C—H linker Ar), 127.83 (s, linker Ar), 118.99 (d, J_{CF}=8.0 Hz, Ar), 116.24 (q, J_{CF}=292.4 Hz, CF₃ of TFA), 110.73 (d, J_{CF}=23.5 Hz, C—H Ar), 106.72 (s, C—H Ar), 105.70 (s, Ar), 59.92 (s, linker CH₂-piperazine), 55.83 (s, linker CH₂-cyclen), 50.91 (s, piperazine CH₂), 47.30 (s, cyclen CH₂), 46.33 (d, J_{CF}=3.8 Hz, piperazine CH₂), 44.21 (s, cyclen CH₂), 41.79 (s, cyclen CH₂), 41.56 (s, cyclen CH₂), 36.05 (s, cyclopropane CH), 7.37 (s, cyclopropane CH₂).

HRMS (ESI+ QTOFMS) calculated for C₃₃H₄₅FN₇O₃ ([M+H]⁺) m/e 606.3562; measured m/e 606.3583.

Compound 5. Following the general procedure, compound 9e (857 mg, 0.92 mmol) yielded 38% over 2 steps (214 mg, 0.35 mmol);

¹H NMR (500 MHz, CD₃OD): δ_{H}=8.58 (s, 1H, QH-2), 7.88 (d, J_{HF}=13.5 Hz, 1H, QH-5), 7.38 (d, J_{HF}=7.0 Hz, 1H, QH-8), 7.16 (d, J=7.6 Hz, 2H, linker Ar), 7.08 (d, J=7.6 Hz, 2H, linker Ar), 3.55-3.50 (m, 1H, cyclopropane CH), 3.49 (bs, 2H, linker CH₂-piperazine), 3.29-3.22 (m, 4H, piperazine 2×CH₂), 2.86-2.80 (m, 4H, cyclen 2×CH₂), 2.80-2.74 (m, 8H, cyclen 4×CH₂), 2.74-2.66 (m, 8H, cyclen 2×CH₂, linker CH₂CH₂-cyclen, linker CH₂CH₂-cyclen), 2.63-2.57 (m, 4H, piperazine 2×CH₂), 1.31-1.24 (m, 2H, cyclopropane CH₂), 1.11-1.02 (m, 2H, cyclopropane CH₂);

¹³C NMR (126 MHz, CD₃OD): δ_{C}=176.71 (s, C═O), 172.44 (s, C═O), 154.51 (d, J_{CF}=247.4 Hz, Ar), 148.39 (s, C—H Ar), 145.83 (d, J_{CF}=10.6 Hz, Ar), 140.61 (s, linker Ar), 140.02 (s, Ar), 135.95 (s, linker Ar), 130.85 (s, C—H linker Ar), 129.74 (s, C—H linker Ar), 123.25 (s, Ar), 117.35 (s, Ar), 113.03 (d, J_{CF}=22.1 Hz, C—H Ar), 106.50 (s, C—H Ar), 63.55 (s, linker CH₂-piperazine), 56.81 (s, linker CH₂CH₂-cyclen), 53.86 (s, piperazine CH₂), 51.30 (s, cyclen CH₂), 50.88 (d, J_{CF}=4.0 Hz, piperazine CH₂), 46.64 (s, cyclen CH₂), 45.13 (s, cyclen CH₂), 44.87 (s, cyclen CH₂), 35.67 (s, cyclopropane CH), 33.87 (s, linker CH₂CH₂-cyclen), 8.51 (s, cyclopropane CH₂).

HRMS (ESI+ QTOFMS) calculated for C₃₄H₄₇FN₇O₃₂ ([M+H]⁺) 620.3719 m/e; measured m/e 620.3687.

Compound 6. Following the general procedure, compound 9f (1.32 gram, 1.39 mmol) yielded 59% over 2 steps (519 mg, 0.82 mmol);

¹H NMR (500 MHz, CD₃OD): δ_{H}=8.59 (s, 1H, QH-2), 7.87 (d, J_{HF}=13.5 Hz, 1H, QH-5), 7.44 (d, J_{HF}=7.3 Hz, 1H, QH-8), 7.06 (d, J=7.9 Hz, 2H, linker Ar), 6.99 (d, J=7.9 Hz, 2H, linker Ar), 3.61-3.53 (m, 1H, cyclopropane CH), 3.43 (bs, 2H, linker CH₂-piperazine), 3.30-3.23 (m, 4H, piperazine 2×CH₂), 2.90-2.80 (m, 12H, cyclen 6×CH₂), 2.70-2.64 (m, 4H, cyclen 2×CH₂), 2.60-2.54 (m, 4H, piperazine 2×CH₂), 2.53-2.45 (m, 4H, linker CH₂(CH₂)₂-cyclen, linker (CH₂)₂CH₂-cyclen), 1.80-1.68 (m, 2H, linker CH₂CH₂CH₂-cyclen), 1.36-1.25 (m, 2H, cyclopropane CH₂), 1.15-1.06 (m, 2H, cyclopropane CH₂);

¹³C NMR (126 MHz, CD₃OD): δ_{C}=176.64 (s, C═O), 172.27 (s, C═O), 154.56 (d, J_{CF}=247.9 Hz, Ar), 148.31 (s, C—H Ar), 145.94 (d, J_{CF}=10.5 Hz, Ar), 142.38 (s, linker Ar), 140.11 (s, Ar), 135.62 (s, linker Ar), 130.52 (s, C—H linker Ar), 129.26 (s, C—H linker Ar), 123.09 (d, J_{CF}=6.4 Hz, Ar), 116.92 (s, Ar), 113.02 (d, J_{CF}=23.1 Hz, C—H Ar), 106.53 (d, J_{CF}=2.9 Hz, C—H Ar), 63.60 (s, linker CH₂-piperazine), 54.96 (s, linker (CH₂)₂CH₂-cyclen), 53.89 (s, piperazine CH₂), 51.55 (s, cyclen CH₂), 50.87 (d, J_{CF}=3.9 Hz, piperazine CH₂), 46.72 (s, cyclen CH₂), 45.09 (s, cyclen CH₂), 44.87 (s, cyclen CH₂), 35.76 (s, cyclopropane CH), 34.25 (s, linker CH₂(CH₂)₂-cyclen), 29.72 (s, linker CH₂CH₂CH₂-cyclen), 8.53 (s, cyclopropane CH₂).

HRMS (ESI+ QTOFMS) calculated for C₃₅H₄₉FN₇O₃ ([M+H]⁺) m/e 634.3875; measured m/e 634.3877.

General Procedure for the Synthesis and Characterization of Aqueous Cu(II)-Complexes:

A stoichiometric amount of CuCl₂(aq) (1.0 mol equivalent) was added to an aqueous solution of the cyclen derivative (Compounds 1-6, 10 mg in 1 mL) and the mixture was stirred at room temperature. UV-VIS spectroscopy (λₘₐₓ 600 nm) was used to follow the progress of the complexation until completion. The solution was then microfiltered and lyophilized to produce the desired complex in quantitative yield. The complexes were characterized by UV-VIS, HRMS and EPR. The EPR spectra of the Cu(II) complexes of the cyclen derivatives 1-6 were identical, and comparable with the simulated spectra and with the literature values for Cu(II)-cyclen [Sawada et al. Biochem. Biophys. Res. Commun. 1995, 216 (1), 154-161]. The solid state (liquid nitrogen) demonstrated the expected axial symmetry with gₚₐᵣₐₗₗₑₗ=2.171 and g_{perpendicular}=2.070 [Aₚₐᵣₐₗₗₑₗ(Cu)=175.0 G]. In water solution, Cu(II) is characterized by the interaction of an unpaired electron with the magnetic isotopes of copper [a(Cu)=73.0 G, (I=3/2) and a g-factor which is strongly shifted (gᵢₛₒ=2.104) from that of a free-electron value (2.0023).

1-Cu(II): UV/Vis (H₂O): λₘₐₓ=600; HRMS (ESI+ QTOFMS) calculated for C₃₁H₄₇CuFN₇O₃ ([M−H]⁺) m/e 647.3015; measured m/e 647.3009.

2-Cu(II): UV/Vis (H₂O): λₘₐₓ=600; HRMS (ESI+ QTOFMS) calculated for C₃₂H₄₉CuFN₇O₃ ([M−H]⁺) m/e 661.3171; measured m/e 661.3128. Analytical purity (HPLC): 93.2% a.

3-Cu(II): UV/Vis (H₂O): λₘₐₓ=598; HRMS (ESI+ QTOFMS) calculated for C₃₃H₅₁CuFN₇O₃ ([M−H]⁺) m/e 675.3328; measured m/e 675.3309.

4-Cu(II): UV/Vis (H₂O): λₘₐₓ=599; HRMS (ESI+ QTOFMS) calculated for C₃₃H₄₃CuFN₇O₃ ([M−H]⁺) m/e 667.2702; measured m/e 667.2693.

5-Cu(II): UV/Vis (H₂O): λₘₐₓ=599; HRMS (ESI+ QTOFMS) calculated for C₃₄H₄₅CuFN₇O₃ ([M−H]⁺) m/e 681.2858; measured m/e 681.2878. Analytical purity (HPLC): 92.7%.

6-Cu(II): UV/Vis (H₂O): λₘₐₓ=600; HRMS (ESI+ QTOFMS) calculated for C₃₅H₄₇CuFN₇O₃ ([M−H]⁺) m/e 695.3015; measured m/e 695.3014.

EPR Spectra: Identical EPR spectra were recorded for Cu(II) complexes of the ligands 1-2 and 4-6 (not determined for 3). The spectra shown below are as follows: (a) Frozen water solutions at 220 K (g_{perpendicular}=2.071; gₚₐᵣₐₗₗₑₗ=2.170, Aₚₐᵣₐₗₗₑₗ(Cu)=175.0 G); (b) simulated spectra of frozen water solutions; (c) water solutions at 350K [a(Cu)=73.0 G, (I=3/2), gᵢₛₒ=2.104] (d) simulated spectra in water. The line width in the Cu(II) spectrum in solution strongly depends on Mi, anisotropy of the g-factor and hyperfine interaction.

General Procedure for the Synthesis and Characterization of Aqua-Hydroxo Co(III)-Complexes:

The syntheses followed a previously reported procedure [Athey et al. J. Org. Chem. 2002, 67(12), 4081-4085] with slight changes and included the following three steps:

Step 1: Synthesis of Co(III)CO₃ complexes as demonstrated by the synthesis of cis-[Co(cyclen)CO₃]HCO₃:

Free-amine cyclen (0.070 gram, 0.406 mmol) was dissolved in a MeOH:H₂O mixture (1:1) (2 mL) and an equimolar amount of Na₃[Co(CO₃)₃]·3H₂O (0.147 gram, 0.406 mmol) was added. The dark green solution was left to react for 16 hours at 65° C. The solution was filtered whilst hot under gravity to separate the liquid from a black solid. The filtrate was dried under vacuum, re-dissolved in MeOH (4 mL), and the resulting solution was filtered to remove a white precipitate. The resulting filtrate was dried under vacuum, re-dissolved in water and then lyophilized to afford a pink powder (0.071 gram, 50%).

¹H NMR (500 MHz, D₂O): δ_{H}=3.60-3.48 (m, 2H, CH₂), 3.15-3.06 (m, 2H, CH₂), 3.02-2.89 (m, 4H, 2×CH₂), 2.88-2.80 (m, 6H, 3×CH₂), 2.77-2.68 (m, 2H, CH₂); ¹³C NMR (126 MHz, D₂O with internal MeOH marker): δ=171.66 (s), 167.24 (s), 162.34 (s) (free HCO₃⁻, free CO₃²⁻, complexed CO₃²⁻), 56.37 (s, CH₂), 53.92 (s, CH₂), 50.40 (s, CH₂), 47.79 (s, CH₂);

UV/Vis (H₂O): λₘₐₓ=517, 362 nm;

ESI+ QTOFMS calculated for C₉H₂₀CoN₄O₃ ([M]⁺) 291.09 m/e; measured 291.11 m/e.

The Co(III)CO₃ complexes of ligands 1-6 were characterized by UV-VIS, ¹³C-NMR and MS. The ¹³C-NMR peaks of the cyclen carbons of the complexes were significantly shifted from the metal-free ligands as observed for the Co(III)-cyclen complex itself; this clearly demonstrated that the Co(III) had become coordinated to the cyclen group as desired.

1-cis-[Co(III)]CO₃: Following the general procedure, compound 1 (0.050 gram, 0.085 mmol) and Na₃[Co(CO₃)₃]·3H₂O (0.030 gram, 0.083 mmol) were heated for 16 hours, and yielded the corresponding Co(I)CO₃ complex as a dark, pink powder (43 mg, 66%).

¹³C NMR (126 MHz, D₂O with internal CD₃OD marker): δ_{C}=176.25, 172.74, 171.85, 167.82, 162.65 (free HCO₃⁻, free CO₃²⁻, complexed CO₃²⁻), 154.93, 153.10, 147.91, 144.91, 139.21, 122.64, 117.39, 112.38, 106.84, 63.45, 58.42, 56.49, 55.44, 53.96, 52.72, 50.42, 49.80, 35.50, 27.31, 26.06, 24.23, 21.62, 8.30;

UV/Vis (MeOH): λₘₐₓ=533;

ESI+ QTOFMS calculated for C₃₂H₄₈CoFN₇O₆ (M⁺) m/e 704.30; measured m/e 704.32.

2-cis-[Co(III)]CO₃: Following the general procedure, compound 2 (0.050 gram, 0.083 mmol) and Na₃[Co(CO₃)₃]·3H₂O (0.031 gram, 0.086 mmol) were heated for 16 hours, and yielded the corresponding Co(III)CO₃ complex as a dark, pink powder (23 mg, 35%).

UV/Vis (MeOH): λₘₐₓ=535;

ESI+ QTOFMS calculated for C₃₃H₅₀CoFN₇O₆ (M⁺) m/e 718.31; measured m/e 718.31.

3-cis-[Co(III)]CO₃: Following the general procedure, compound 3 (0.070 gram, 0.114 mmol) and Na₃[Co(CO₃)₃]·3H₂O (0.041 gram, 0.114 mmol) were heated for 16 hours, and yielded the corresponding Co(III)CO₃ complex as a dark, pink powder (55 mg, 61%).

¹³C NMR (75 MHz, D₂O with internal CD₃OD marker): δ_{C}=176.07, 172.56, 171.95, 167.81, 165.59 (free HCO₃⁻, free CO₃²⁻, complexed CO₃²⁻), 155.50, 151.24, 122.62, 116.96, 112.3, 106.91, 63.38, 59.04, 58.14, 56.51, 55.44, 53.96, 53.17, 50.40, 29.73, 27.92, 27.58, 26.52, 21.81, 8.34;

UV/Vis (MeOH): λₘₐₓ=535;

ESI+ QTOFMS calculated for C₃₄H₅₂CoFN₇O₆ (M⁺) m/e 732.33; measured m/e 732.32.

4-cis-[Co(III)]CO₃: Following the general procedure, compound 4 (0.050 gram, 0.083 mmol) and Na₃[Co(CO₃)₃]·3H₂O (0.030 gram, 0.083 mmol) were heated for 16 hours, and yielded the corresponding Co(III)CO₃ complex as a dark, pink powder (49 mg, 77%).

¹³C NMR (75 MHz, D₂O with internal CD₃OD marker): δ_{C}=174.90, 171.94, 170.99, 166.92, 164.37 (free HCO₃⁻, free CO₃²⁻, complexed CO₃²⁻), 154.40, 151.11, 146.77, 143.64, 138.21, 136.50, 132.41, 130.17, 128.99, 121.58, 116.45, 111.38, 105.86, 63.03, 61.46, 56.67, 55.76, 53.15, 51.73, 48.01, 47.47, 47.02, 34.50, 7.36;

UV/Vis (MeOH): λₘₐₓ=533;

ESI+ QTOFMS calculated for C₃₄H₄₄CoFN₇O₆ (M⁺) m/e 724.27; measured m/e 724.27.

5-cis-[Co(III)]CO₃: Following the general procedure, compound 5 (0.057 gram, 0.092 mmol) and Na₃[Co(CO₃)₃]·3H₂O (0.033 gram, 0.092 mmol) were initially heated for 16 hours. ¹³C NMR revealed that the reaction was incomplete and that a significant amount of starting material remained. The addition of another equivalent of Na₃[Co(CO₃)₃]·3H₂O and heating for a further 24 hours ensured that all of the starting material was consumed, and yielded a dark, pink powder (30 mg, 41%).

¹³C NMR (150 MHz, D₂O with internal CD₃OD marker): δ_{C}=182.42, 175.99, 173.02, 171.97, 167.70, 166.12 (free HCO₃⁻, free CO₃²⁻, complexed CO₃²⁻), 154.76, 153.13, 147.83, 144.83, 139.32, 138.23, 134.83, 131.40, 129.70, 122.79, 117.61, 112.25, 107.13, 63.68, 62.61, 58.55, 56.77, 56.57, 54.02, 52.72, 50.43, 50.14, 49.75, 48.36, 35.58, 28.06, 24.23, 8.39;

UV/Vis (MeOH): λₘₐₓ=533;

ESI+ QTOFMS calculated for C₃₅H₄₆CoFN₇O₆ (M⁺) m/e 738.28; measured m/e 738.27.

6-cis-[Co(III)]CO₃: Following the general procedure, compound 6 (0.075 gram, 0.118 mmol) and Na₃[Co(CO₃)₃]·3H₂O (0.043 gram, 0.118 mmol) were heated for 16 hours, and yielded the corresponding Co(III)CO₃ complex as a dark, pink powder (55 mg, 57%).

¹³C NMR (75 MHz, D₂O with internal CD₃OD marker): δ_{C}=175.78, 172.72, 171.92, 167.73, 163.68 (free HCO₃⁻, free CO₃²⁻, complexed CO₃²⁻), 155.33, 152.05, 147.90, 144.68, 141.58, 139.15, 134.13, 131.09, 129.39, 122.47, 117.30, 112.17, 106.65, 63.41, 62.64, 58.35, 56.57, 54.88, 54.02, 52.85, 50.40, 49.83, 49.59, 48.40, 35.45, 33.23, 23.91, 8.39;

UV/Vis (MeOH): λₘₐₓ=529;

ESI+ QTOFMS calculated for C₃₆H₄₈CoFN₇O₆ (M⁺) m/e 752.30; measured m/e 752.29.

Step 2 and Step 3: Synthesis and activation of Co(III)Cl₂ complexes as demonstrated by the synthesis and activation of cis-[Co(cyclen)Cl₂]Cl:

The cis-[Co(cyclen)CO₃]HCO₃ (0.071 gram, 0.270 mmol) was dissolved in MeOH (3 mL), and then HCl (10.2 M, 1.5 mL) was added gradually. The reaction crude was dried under vacuum and the residue was re-dissolved in MeOH (3 mL), treated with HCl (10.2 M, 1.5 mL) and then reduced to dryness. This procedure was repeated once more and a gradual color change from dark pink to dark violet was observed. The dry residue was then re-dissolved in MeOH (5 mL), placed into an ice bath and then ether added (45 mL). The resulting precipitate was filtered under gravity using a sintered glass funnel and then washed multiple times with ether until the filtrate was pH-neutral, to afford a violet solid in quantitative yield.

¹³C NMR (126 MHz, D₂O with internal MeOH marker): δ=57.34 (s, CH₂), 54.21 (s, CH₂), 49.60 (s, CH₂), 46.23 (s, CH₂);

UV/Vis (DMSO): λₘₐₓ=560, 380 nm.

A 5 mM stock solution of the dichloride complex was prepared in HEPES (10 mM, pH 7.6) and then activated to the catalytically active aqua-hydroxo species by addition of two equivalents of 0.1 M NaOH at room temperature, as shown by the immediate change in the visible absorption band from 560 nm to 519 nm.

The Co(III)Cl₂ complexes of ligands 1-6 were characterized by UV-VIS and HRMS.

1-cis-[Co(III)]Cl₂: Following the general procedure, 1-cis-[Co(III)]CO₃ (0.043 gram, 0.056 mmol) yielded the corresponding Co(III)Cl₂ complex as a violet powder in quantitative yield.

UV/Vis (MeOH): λₘₐₓ=561.

Following the general procedure, the dichloride complex was activated with base treatment as shown by the immediate change in the visible absorption band from 561 nm to 524 nm.

2-cis-[Co(III)]Cl₂: Following the general procedure, 2-cis-[Co(III)]CO₃ (0.023 gram, 0.029 mmol) yielded the corresponding Co(III)Cl₂ complex as a violet powder in quantitative yield.

UV/Vis (MeOH): λₘₐₓ=565.

Following the general procedure, the dichloride complex was activated with base treatment as shown by the immediate change in the visible absorption band from 565 nm to 528 nm.

3-cis-[Co(III)]Cl₂: Following the general procedure, 3-cis-[Co(III)]CO₃ (0.051 gram, 0.064 mmol) yielded the corresponding Co(III)Cl₂ complex as a violet powder in quantitative yield.

UV/Vis (MeOH): λₘₐₓ=565;

HRMS (ESI+ QTOFMS) calculated for C₃₃H₅₂Cl₂CoFN₇O₃ (M⁺) m/e 742.2819; measured m/e 742.2796.

Following the general procedure, the dichloride complex was activated with base treatment as shown by the immediate change in the visible absorption band from 565 nm to 528 nm.

4-cis-[Co(III)]Cl₂: Following the general procedure, 4-cis-[Co(III)]CO₃ (0.0455 gram, 0.059 mmol) yielded the corresponding Co(III)Cl₂ complex as a violet powder in quantitative yield.

UV/Vis (MeOH): λₘₐₓ=575;

HRMS (ESI+ QTOFMS) calculated for C₃₃H₄₄Cl₂CoFN₇O₃ (M⁺) m/e 734.2193; measured m/e 734.2171.

Following the general procedure, the dichloride complex was activated with base treatment as shown by the immediate change in the visible absorption band from 575 nm to 533 nm.

5-cis-[Co(III)]Cl₂: Following the general procedure, 5-cis-[Co(III)]CO₃ (0.030 gram, 0.0375 mmol) yielded the corresponding Co(III)Cl₂ complex as a violet powder (20 mg, 68%).

UV/Vis (MeOH): λₘₐₓ=564.

Following the general procedure, the dichloride complex was activated with base treatment as shown by the immediate change in the visible absorption band from 564 nm to 525 nm. 6-cis-[Co(III)]Cl₂: Following the general procedure, 6-cis-[Co(III)]CO₃ (0.055 gram, 0.0676 mmol) yielded the corresponding Co(III)Cl₂ complex as a violet powder (53 mg, 98%).

UV/Vis (MeOH): λₘₐₓ=568;

HRMS (ESI+ QTOFMS) calculated for C₃₅H₄₈Cl₂CoFN₇O₃ (M⁺) m/e 762.2506; measured m/e 762.2503.

Following the general procedure, the dichloride complex was activated with base treatment as shown by the immediate change in the visible absorption band from 568 nm to 527 nm.

### Example 2

### DNA Cleavage Assays

To evaluate the biologically pertinent nuclease activity of Cu(II) and Co(III) complexes of Compounds 1-6, assays were performed (a) in the absence of redox adjuvants and (b) in the presence of 0.32 mM ascorbic acid (for the Cu(II) complexes), since its (eukaryotic) intracellular concentration is also in this range [Hormann et al. Eur. J. Inorg. Chem. 2015, 2015 (28), 4722-4730; Kashiba-Iwatsuki et al. FEBS Lett. 1996, 389 (2), 149-15]. Agarose gel electrophoresis (1% agarose) and ethidium bromide staining was used to monitor the conversion of supercoiled (Form-I) pHOT-1 plasmid DNA into its nicked form (Form-II) or into multiply nicked DNA; no linear form (Form-III) was observed. Comparative DNA cleavage experiments of the ligands 1-6 with and without the chelated metals clearly demonstrated that the cleavage activity of Compounds 1-6 without any metal could be excluded, as shown in FIG. 6. Due to the presence of multiple positive charges in the ligands it is necessary to use an ion-exchange based microscale procedure for removal of the ligands prior to electrophoresis (above a critical concentration; data not shown), as neutralization of the negatively charged DNA by these strongly bound ligands prevents the DNA from moving in the electric field.

Cleavage Experiments in the Absence of Adjuvants—Cu(H) Complexes:

As illustrated in FIG. 7A, both the 1-Cu(II) and 4-Cu(II) complexes show very significant enhancement in the rates of DNA cleavage when compared with either CuCl₂ or Cu(II)-cyclen. Presumably, this enhancement is mediated by the extra binding affinity provided by the intercalating properties of the ciprofloxacin scaffold. As shown in FIG. 7B, both 2-Cu(II) and 5-Cu(II) complexes show significant cleavage enhancement even in relation to the activity of 1-Cu(II) and 4-Cu(II). There appears to be a strong correlation between the linker length and the cleavage activity; 1-Cu(II) and 4-Cu(II) show similar activity and both have six atoms in the linker, whilst 2-Cu(II) and 5-Cu(II) also show similar activity and both have seven atoms in the linker. As shown in FIG. 7C, 3-Cu(II) and 6-Cu(II) both of which contain eight atoms in the linker, showed only very limited activity. The overall order of reactivity is therefore as follows: 2-Cu(II)≈5-Cu(II)>1-Cu(II)≈4-Cu(II)>>3-Cu(II)≈6-Cu(II). These data demonstrate that the nuclease activity is very sensitive to the linker length between the Cu(II)-cyclen warhead and the ciprofloxacin scaffold.

To investigate the mechanism of cleavage, a scavenging assay was performed with hydroxyl radical scavengers (DMSO, t-BuOH, KI), a singlet oxygen scavenger (NaN₃), a superoxide scavenger (KI) and with NaCl as a control for ionic strength. The obtained data is presented in FIG. 7D and clearly rules out the involvement of the hydroxyl radical in the catalytic activity of 5-Cu(II), since both DMSO and t-BuOH have no significant inhibitory effect.

KI and NaN₃ both have a significant inhibitory effect whilst NaCl does not, ostensibly suggesting that both singlet oxygen and superoxide radicals contribute to the catalytic activity of 5-Cu(II). Other studies have reported on similar copper complexes (bearing DNA intercalators) that cleave DNA via an oxidative pathway in the absence of adjuvants [Liu et al. Eur. J. Med. Chem. 2010, 45 (11), 5302-5308; Zhang et al. Sci. China Chem. 2011, 54 (1), 129-136].

Since the inhibitory effects of NaN₃ and KI could also be mediated by their ability to displace the copper-coordinated water, these data do not rule out the possibility that a hydrolytic mechanism contributes to the nuclease activity.

UV-VIS spectroscopic analysis of the mixture of Cu(II)-cyclen with NaN₃, KI or NaCl (data not shown) reveal that NaN₃ and KI displace the Cu(II) associated water whilst the NaCl does not (i.e. at the ratio used in the scavenging assay).

The fact that one additional carbon in the linker can distinguish excellent nuclease activity enhancement (relative to Cu(II)-cyclen) from reduced or nullified activity [i.e. 5-Cu(II) and 2-Cu(II) vs. 3-Cu(II) and 6-Cu(II)] suggests that a hydrolytic mechanism contributes significantly to the nuclease activity of 1-Cu(II), 2-Cu(II), 4-Cu(II) and 5-Cu(II).

Cleavage Experiments in the Absence of Adjuvants—Co(III) Complexes:

As shown in FIG. 8, the Co(III) complexes of ligands 1-6 all cause the plasmid DNA to ‘disappear’ during the incubation experiment at micromolar concentrations but do not generate any new DNA band or DNA smear. This is in contrast to Co(III)-cyclen itself which has no effect whatsoever under these conditions. Increasing the incubation time from 0.5 to 2 hours had no significant effect on the results (data not shown), suggesting that the complete disappearance of DNA is caused by a binding event rather than multiple cleavage events. These data collectively suggest that the Co(III) complexes of ligands 1-6 possess significant hydrolytic DNase activity at micromolar concentrations, but that the ligand-metal sequestering procedure used successfully for the Cu(II) complexes was unable to remove the Co(III) complexes from the DNA; presumably this in-turn causes the DNA to (i) be charge neutralized and consequently not to run on the gel and/or (ii) prevents the ethidium bromide from binding to the DNA.

These data demonstrate that the Co(III) complexes of the compounds 1-6 bind very strongly to DNA and therefore would not be able to generate a catalytic turnover. Without being bound by any particular theory, it is assumed that the Co(III) complexes of 1-6 form cyclic phosphate as the only hydrolysis product, which is known to be particularly stable for Co(III).

Cleavage in the Presence of Ascorbic Acid—Cu(II) Complexes:

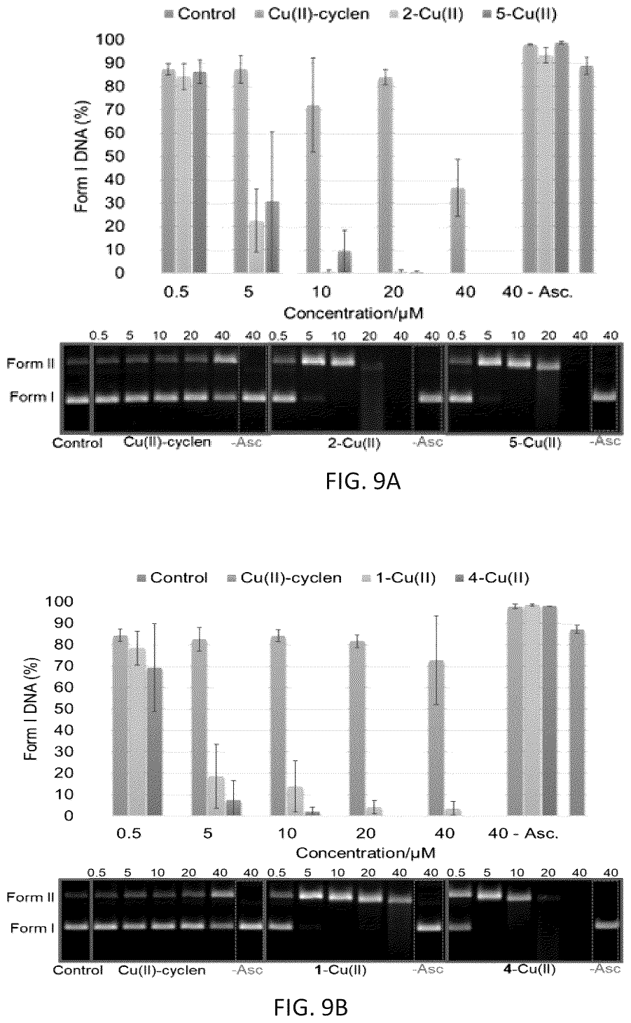

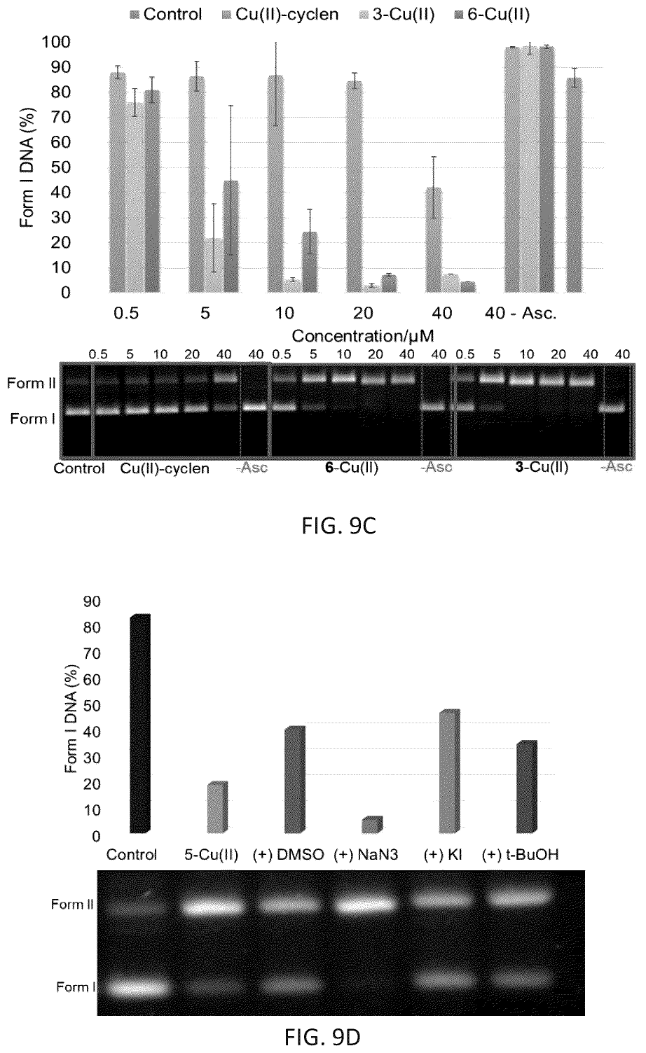

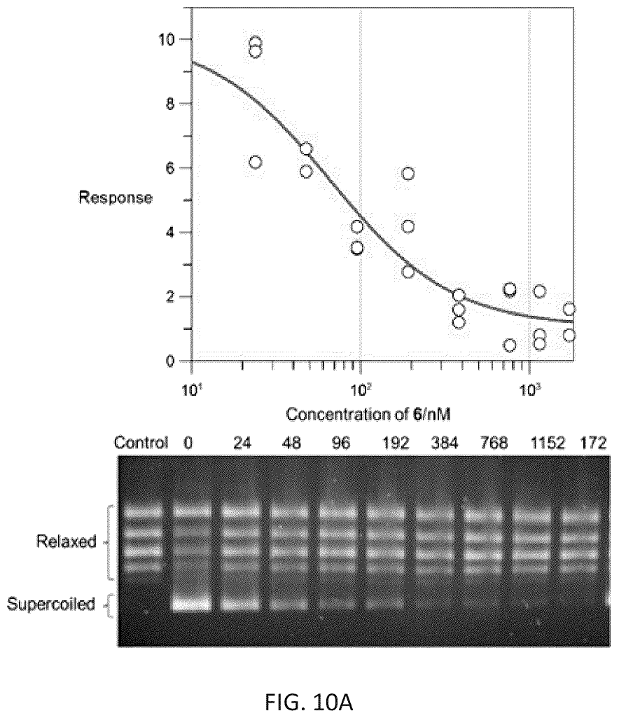

As shown in FIGS. 9A-C, the Cu(II) complexes of ligands 1-6 in the presence of ascorbic acid, all show very significant cleavage enhancement when compared with Cu(II)-cyclen. Presumably, the nuclease activity enhancement versus that of Cu(II)-cyclen is once again mediated by the extra binding affinity provided by the intercalation of the ciprofloxacin scaffold in 1-6 with the DNA. The concentration dependent conversion of form I to form II is similar for all the complexes but the conversion of form II to multiply nicked DNA (as evidenced by a DNA smear) varies considerably. The most active compound 2-Cu(II) shows almost complete smearing of the DNA from 20 μM (see, FIG. 9A), while 1-Cu(II) shows only minimal smearing even at 40 μM (see FIG. 9B). These data suggest that while the cleavage ability of all the ligands is quite similar (i.e. k₂), the turnover frequency varies considerably.

To investigate the mechanism of cleavage, a scavenging assay was performed with hydroxyl radical scavengers (DMSO, t-BuOH, KI), a singlet oxygen scavenger (NaN₃), and a superoxide scavenger (KI). The obtained data is presented in FIG. 9D, and show that all the hydroxyl scavengers significantly inhibited the cleavage, NaN₃ showed no inhibition whatsoever, and KI did not show significantly more inhibition than DMSO or t-BuOH. These data suggest that in the presence of ascorbic acid, 5-Cu(II) predominantly mediates DNA cleavage via the hydroxy radical mechanism.

### Example 3

### Escherichia coli DNA Gyrase Assays

To evaluate the biological effects and mechanism of the metal-free ligands 1-6 and their Cu(II) complexes on the topoisomerase IIA enzyme system (in vitro), two types of assays were performed: (i) An E. coli DNA gyrase supercoiling inhibition assay (ATP-dependent) and (ii) an E. coli DNA gyrase-induced DNA cleavage assay (ATP-independent).

Metal-Free Ligands:

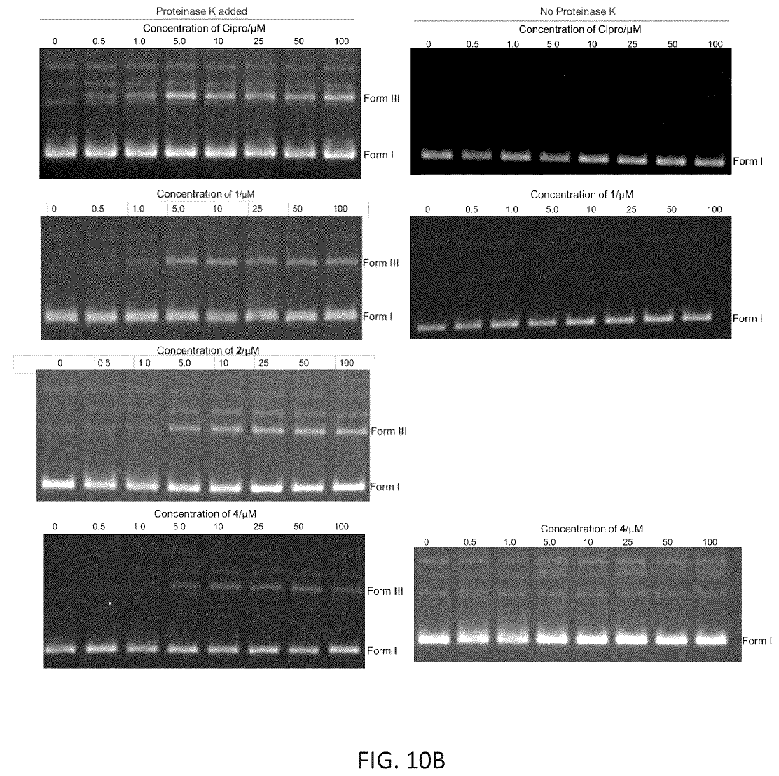

The metal-free ligands 1-2 and 4-6 were tested for their in-vitro activity in a Gyrase inhibition assay in which the supercoiling functionality of the enzyme was measured as a function of compound concentration in the presence of ATP. The measured IC₅₀ values shown in Table 2 in Example 7 below demonstrate that these compounds strongly inhibit DNA gyrase with a similar potency to the parent compound ciprofloxacin. FIG. 10A presents exemplary results obtained for compound 6.

To investigate the mechanism of inhibition, a DNA gyrase-induced DNA cleavage assay was performed in the absence of ATP. In this assay, the production of linear DNA from supercoiled DNA is measured as a function of compound concentration. Incubation of ciprofloxacin or compounds 1-6 with DNA gyrase and DNA generates nicked DNA strands that are covalently linked to the active-site tyrosine residues of the enzyme DNA gyrase. Consequently, for the purposes of this assay, it is necessary to perform a second incubation of the reaction mixture with SDS and Proteinase K, which enables the digestion of the DNA gyrase enzyme and thereby ‘frees’ the linear DNA (Form III) so that it moves during electrophoresis. As shown in FIG. 10B, the tested compounds (1, 2 and 4) exhibited significant linearization of DNA, but only after treatment with Proteinase K, which demonstrates their ability to stabilize the ternary complex in a comparable manner to the parent compound ciprofloxacin.

Cu(II)-complexes: The Cu(II) complexes of ligands 1-2 and 4-6 were initially tested in the DNA gyrase inhibition assay. The measured IC₅₀ data is shown in Table 2 in Example 7 below and demonstrate that the Cu(II)-complexes strongly inhibit DNA gyrase and with a very similar potency to the metal-free ligands.

To investigate the mechanism of inhibition, the DNA gyrase cleavage assay was initially performed in the presence of Proteinase K, with compounds 1-Cu(II), 2-Cu(II) and 4-Cu(II). The obtained data is shown in FIGS. 11A-B and demonstrate that all the tested Cu(II) complexes linearized the DNA in a similar manner to the metal-free ligands in the presence of Proteinase K.

The DNA gyrase cleavage assay was then performed without the Proteinase K treatment, with ciprofloxacin, 1-Cu(II), 2-Cu(II) and 4-Cu(II). It is noted that if these complexes do in-fact cleave DNA, the ternary complex is likely to be destabilized and consequently lead to the release of linear DNA without needing to incubate with Proteinase K, as schematically depicted in FIGS. 12A-B.

As also shown in FIGS. 11A-B, 1-Cu(II) was not able to generate linear DNA at any concentration, like the parent compound ciprofloxacin. However, 4-Cu(II) and 2-Cu(II), were both able to generate a significant quantity of linear DNA and exhibited a well-defined, bell-shaped kinetic profile with a maximum at 25 μM. This is in contrast to the DNase activity of 4-Cu(II) or 2-Cu(II) on plasmid DNA, shown in FIGS. 6 and 9A-C, which show concentration-dependent cleavage and no generation of linear DNA. Consequently, the observed data in FIGS. 11A-B demonstrate that both 4-Cu(II) and 2-Cu(II) cleave DNA from within the ternary complex and subsequently cause it to become destabilized.

### Example 4

### Vulnerability of Cu(H)-Cyclen Under Physiological Conditions

While the data presented herein, particularly for 4-Cu(II) and 2-Cu(II), is indicative of potential catalytic activity of these complexes, the following was observed:

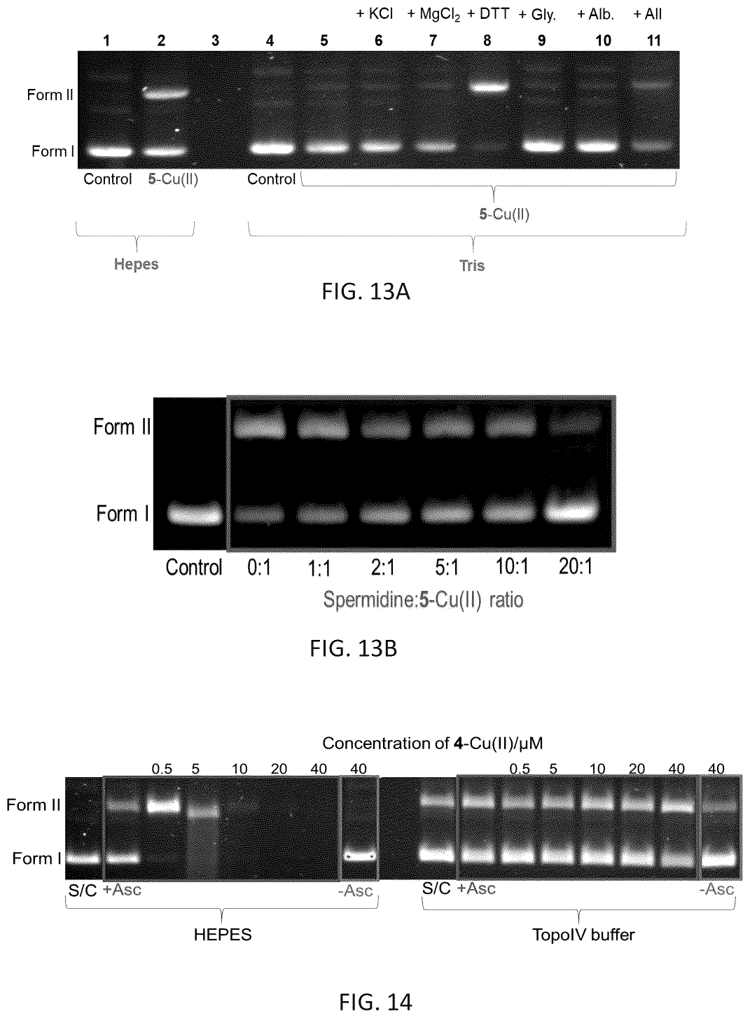

As shown in FIGS. 13A-B, DNA cleavage experiments showed that two of the ingredients in the DNA gyrase cleavage assay, Tris-buffer and spermidine, inhibit the hydrolytic DNA cleavage of plasmid DNA even for the most potent hydrolytic complex, 5-Cu(II). Without being bound by any particular theory, it is assumed that a primary amine of Tris and of spermidine readily exchanges with the Cu(II)-associated water and thereby prevents the formation of the pre-catalytic complex between the DNA backbone and the Cu(II) complex (e.g., as shown in FIG. 3). As shown in FIG. 13A, 5-Cu(II) was observed to retain significant oxidative cleavage activity in Tris buffer in the presence of the reducing agent DTT, an ingredient in the enzyme buffer. These data suggest that under the conditions of the DNA gyrase cleavage assay (see, FIGS. 11A-B), the ternary-complex-destabilization effect of 4-Cu(II) and 2-Cu(II) is mediated solely by oxidative DNA cleavage and not by hydrolytic DNA cleavage.

In addition, as shown in Table 2 below, a similarity was found between the IC₅₀ values for the metal-free and Cu(II) complexes of 2 and 4, even though the respective assay (FIGS. 10A-B) was performed in the presence of DTT.

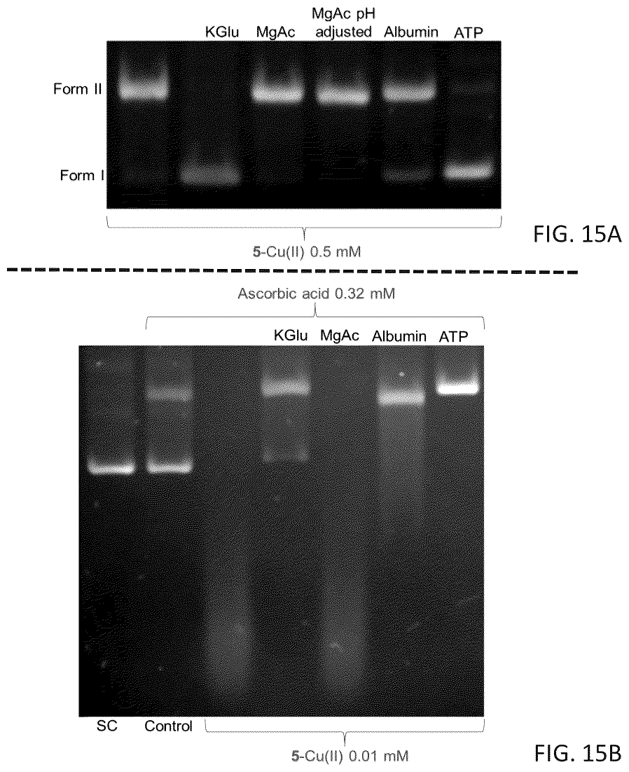

Preliminary experiments performed for a different assay (TopoIV-induced DNA cleavage assay) revealed that ATP (one of the ingredients in the DNA gyrase supercoiling assay/Topo IV cleavage assay but not in the DNA gyrase cleavage assay) strongly inhibits both the hydrolytic and oxidative activity of the Cu(II) complexes of 1-6, as shown in FIGS. 14 and 15A-B, suggesting an explanation for the apparent lack of oxidative DNase activity in the gyrase supercoiling assay.

These experiments also revealed that potassium glutamate (another ingredient of the Topo IV buffer) also strongly inhibits all DNase activity of the Cu(II) complexes of 1-6.

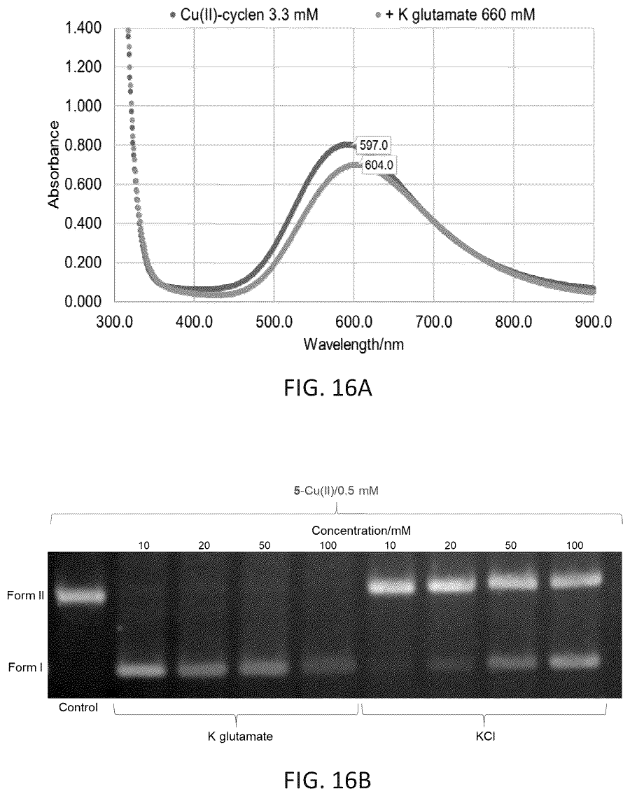

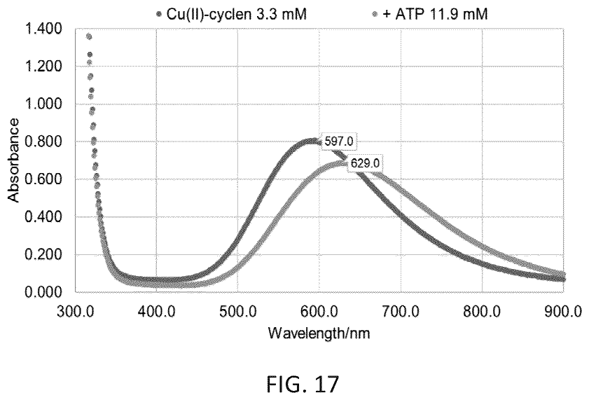

As shown in FIGS. 16A-B and 17, subsequent UV-VIS spectroscopic analysis of the mixture of Cu(II)-cyclen with potassium glutamate or ATP suggested that each of these ingredients inactivates the Cu(II)-cyclen warhead by displacement of the Cu(II)-associated water, thereby eliminating its potential DNase activity. These results also provide an explanation for the unexpected similarity between the MIC data for the Cu(II) complexes of 2 and 4 and their corresponding metal-free ligands (see, Table 2), since free ATP and glutamate are present in bacterial cells at concentrations comparable to the enzyme buffer.

The observed data suggest that at least two of the compounds (i.e. 4-Cu(II) and 2-Cu(II)) are in principle (i.e. in vitro) able to fragment the bacterial chromosome into linear DNA in the presence of bacterial topoisomerase IIA enzyme, via DNA cleavage and subsequent destabilization of the ternary complex.

These data also demonstrate the “vulnerability” of the Cu(II)-cyclen system in vivo, since it is assumed that the natural amino acids along with other substances containing primary amines, and endogenous metal chelators, such as ATP and glutamate, have the potential to bind and inactivate the Cu(II)-cyclen moiety.

### Example 5

### Additional Design and Syntheses

To address the vulnerability issue of the Cu(II)-cyclen system, the inclusion of a dynamic, intramolecular cap in the so called ‘second coordination sphere’ of the complexes was envisioned. Such dynamic ligand exchange systems can facilitate DNA cleavage catalysis and also prevent irreversible ligand exchange reactions, thereby ensuring the integrity of the Cu(II)-cyclen catalytic system in a biological setting.

While relying on previous studies [Sheng, X. et al. Chem.—A Eur. J. 13, 9703-9712 (2007); Hettich, R. & Schneider, H.-J., J. Am. Chem. Soc. 119, 5638-5647 (1997); Tjioe, L. et al., Inorg. Chem. 51, 939-953 (2012)] which showed that several synthetic Cu(II) and Co(III) polyamines exhibit cooperativity of the metal ion center with pendant amine or guanidine functional groups in the cleavage of plasmid DNA, introducing positively charged groups in side chains of the ligands was conceived.

FIG. 18 presents the chemical structures of exemplary ligand structures which feature a guanidine-containing moiety as an exemplary “protecting” moiety that may be coordinated in a dynamic equilibrium with the metal ion, in a reversible manner, so as to protect its poisoning by cellular and other physiological components. Such a moiety is also referred to herein as a moiety that comprises a heteroatom-containing group that has a pKa of from 6 to 8 (e.g., is non-protonated or not fully protonated at physiological pH) and is capable of reversibly binding to the metal ion (e.g., in physiological environment), wherein the heteroatom-containing group is guanidine.

It has been assumed that in order to increase the likelihood of an efficient catalytic turnover, the linker should be designed using molecular docking studies to ensure that the warheads bind ‘on target’ (to the DNA and not to the protein) and interact with the DNA in a catalytically viable manner.

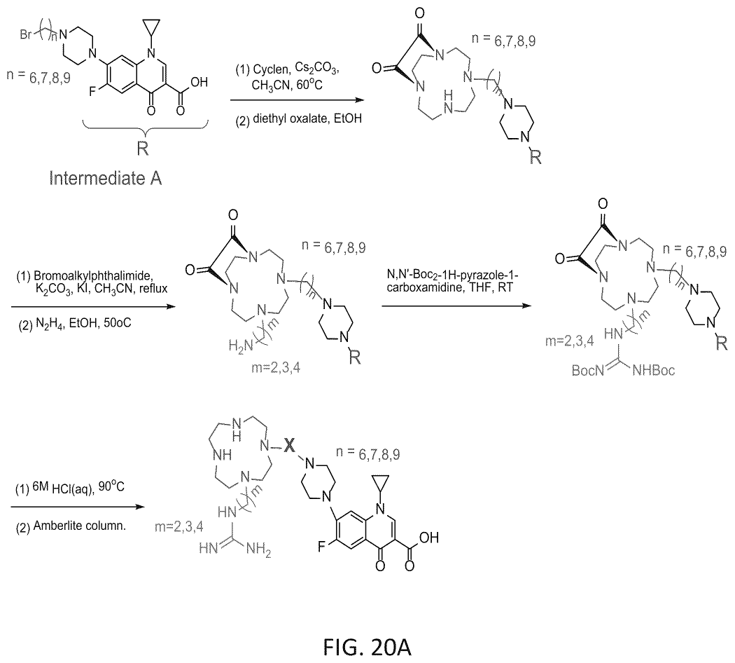

As shown in FIG. 19, a docking pose illustrates one of the compounds with a guanidine-containing pendant moiety at the ortho position as described herein. As depicted, the guanidine warhead is simultaneously proximal to the 3′O of the ribose (potential leaving group) and the phosphate oxygen, while the copper activated nucleophile (water) is well orientated (angle 141°) for an in-line nucleophilic attack. From this docking data, it can also be deduced that if a guanidine-containing pendant moiety would be positioned para to the ciprofloxacin scaffold, it may be too far to provide phosphate oxygen or leaving group stabilization. These preliminary docking and modellings therefore suggested that a guanidine-containing side-chain location ortho to the ciprofloxacin scaffold may be preferred. The guanidine-containing moiety (or any other “protecting” group as described herein) can be attached through the linker (see, upper structures in FIG. 18) or directly to the cyclen moiety (see, lower structures in FIG. 18).

In these compounds, the linker has attached thereto two different functionalities, cyclen and guanidinium.

In attempts to shorten the synthetic steps, a catalytic hydro-amination reaction of un-activated olefins using Iridium photocatalyst under blue LED light has been considered [Musacchio et al. Science (80-.). 355, 727-730 (2017)]. The reaction is redox-neutral, exhibits broad functional group tolerance, and occurs rapidly at room temperature under visible light irradiation.

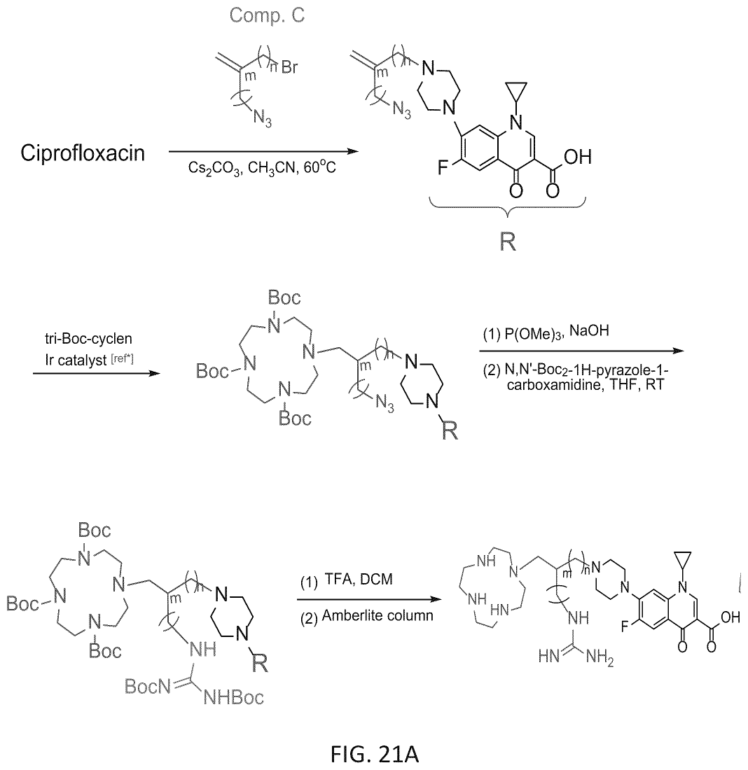

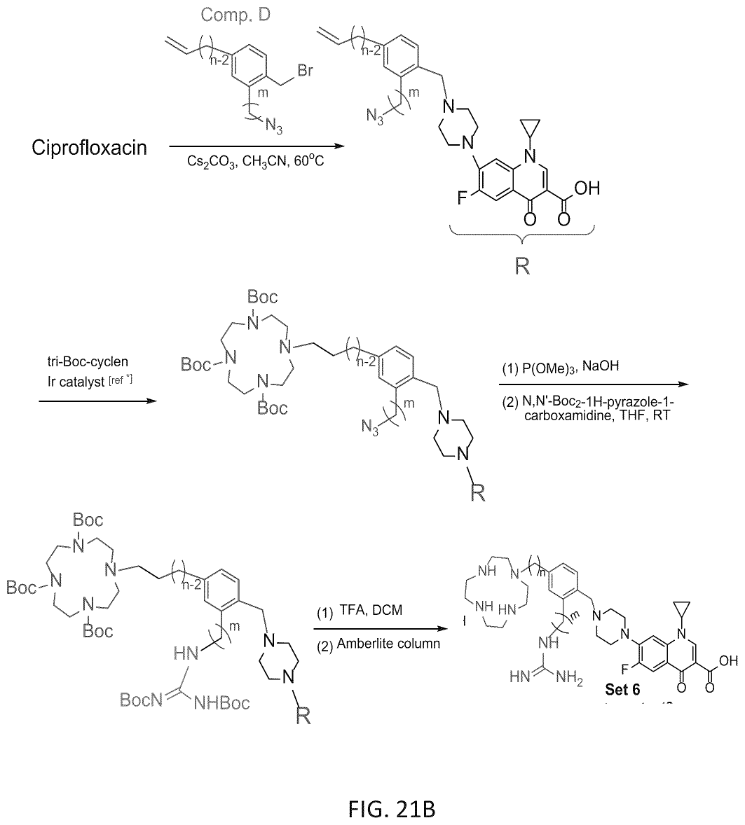

The exemplary compounds shown in FIG. 18 upper row are prepared as exemplified in FIGS. 21A-B.

Briefly, ciprofloxacin is reacted with compound C under base, which after hydro-amination with tri-Boc-cyclen as mentioned above, afford the desired azides. Staudinger reaction is then followed by the introduction of the protected guanidine. Deprotection under acid and ion-exchange column affords the desired structures that feature an aliphatic linker (FIG. 21A). A very similar sequence of steps using compound D provide the desired structures that feature an aromatic linker (FIG. 21B). The appropriately protected olefins, compounds C and D, are prepared by using standard synthetic protocols.

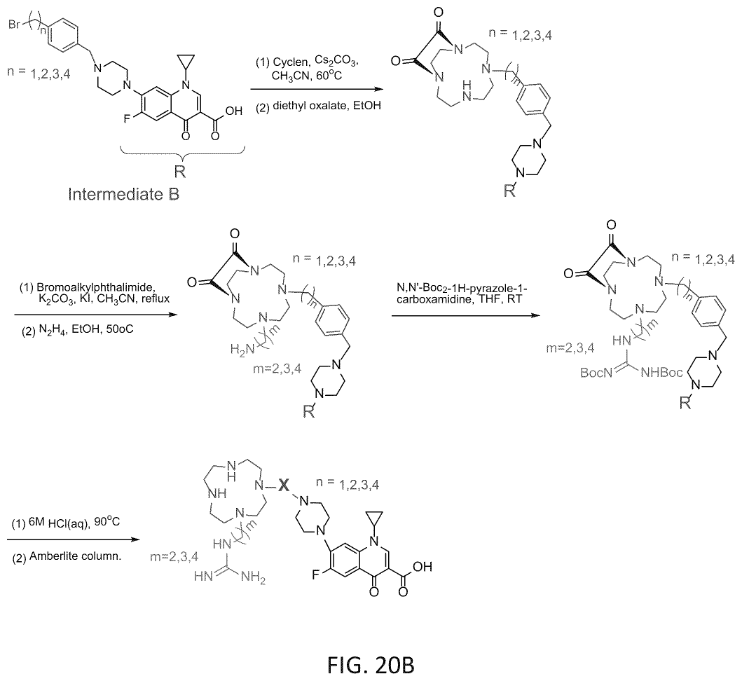

The exemplary compounds shown in FIG. 18 lower row are prepared as exemplified in FIGS. 20A-B.

The intermediate primary bromide derivatives of ciprofloxacin, are used as starting materials, and are reacted with unprotected cyclen under base conditions to afford the corresponding ciprofloxacin-cyclen derivatives. Treatment with diethyl oxalate affords protected intermediates. The remaining free nitrogen of the cyclen is then reacted with bromoalkylphtalimide under base, followed by treatment with hydrazine to yield the desired primary amines. The primary amines are then converted to the corresponding Boc-protected guanidine derivatives according to a known protocol [Tjioe, L. et al. Inorg. Chem. 51, 939-953 (2012)]. Complete deprotection of the Boc-protected guanidine derivatives is accomplished by treatment under concentrated HCl, followed by ion-exchange column to afford the desired compounds in their free base forms.

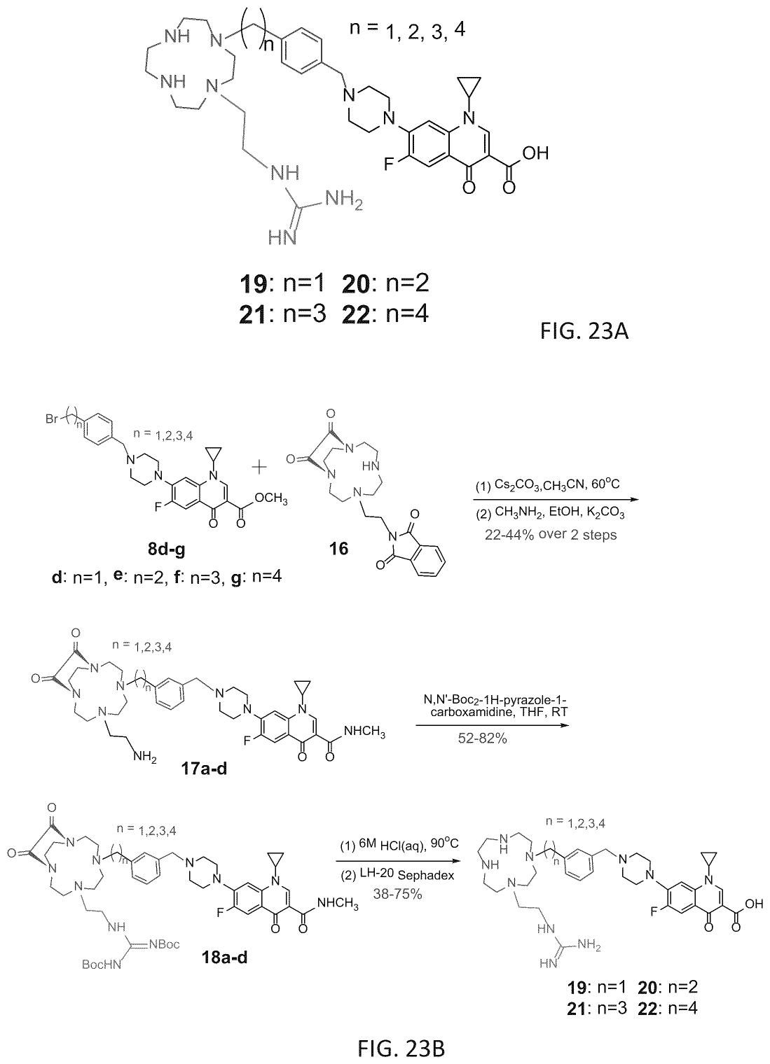

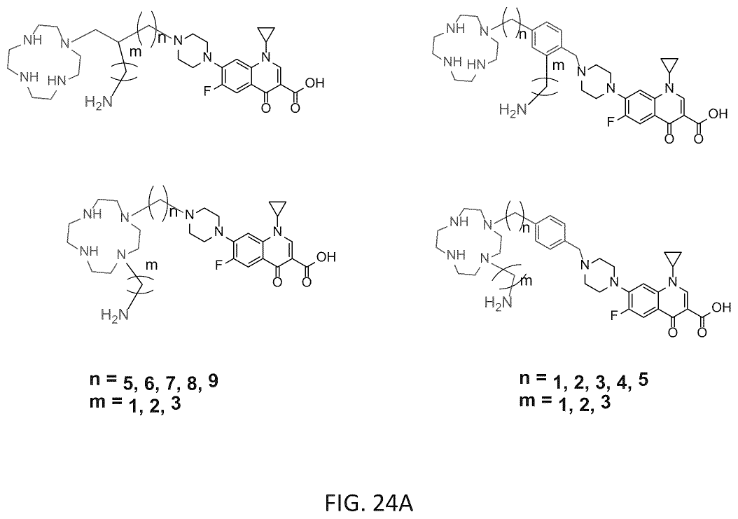

The exemplary tested compounds 19-22 (FIG. 23A) represent the compounds shown in FIG. 18 right lower row, wherein m=2 and n=1-4, respectively. These compounds were prepared using the following procedure (schematically presented in FIG. 23B), which is based on the procedure also described in FIG. 20B.

Synthesis of Ciprofloxacin-Cyclen Conjugates with Guanidine-Containing Pendant Moieties (See, FIG. 23B):

The coupling of primary bromides 8d-g with a protected cyclen 16 under basic conditions (in the presence of, e.g. Cs₂CO₃) provides the corresponding phthalimide derivatives, which after treatment with alkylamine (such as, e.g., methylamine) provides the corresponding amines 17a-d. The amines are then treated with an N-protecting group reagent (such as, e.g. N,N-di-Boc-1H-pyrazole-1-carboxamide) in the presence of a base (such as, e.g. K₂CO₃) to afford the corresponding protected guanidine derivatives 18a-d. Deprotection (using, e.g. 6 M HCl at 90° C.), followed by purification of the crude products (using, e.g. LH-20 size-exclusion column) provides the desired compounds 19-22 with guanidinium pendants at ortho position on the cyclen warhead.

The syntheses and analyses of compounds 8d-f are described in Example 1 hereinabove and are shown in FIG. 5B.

Preparation of Compound 8g:

The methyl ester of ciprofloxacin (8.38 grams, 24.8 mmol) and DIPEA (17 mL, 4 equivalents) were suspended in dry acetonitrile (250 mL). The reaction was stirred in an ice bath under an argon atmosphere, 1-(bromomethyl)-4-(4-bromobutyl) benzene was added (9.08 grams, 1.2 equivalents) and stirring was continued under reduced temperature. Thin layer chromatography (TLC) analysis (dichloromethane/methanol 9:1) indicated complete consumption of the methyl ester of ciprofloxacin after 10 hours. The solvent was evaporated, and the residue was dried under vacuum overnight. The crude product was then loaded onto a dichloromethane-packed silica column as a dichloromethane solution; the desired product was eluted in 3% methanol in dichloromethane to yield 8g as a white solid (8.22 grams, 14.4 mmol, 58%).

¹H NMR (500 MHz, CDCl₃): δ_{H}=8.43 (s, 1H, QH-2), 7.88 (d, J_{HF}=13.4 Hz, 1H, QH-5), 7.25 (d, J=8.0 Hz, 2H, linker Ar), 7.20 (d, J_{HF}=7.1 Hz, 1H, QH-8), 7.13 (d, J=8.0 Hz, 2H, linker Ar), 3.86 (s, 3H, OCH₃), 3.54 (s, 2H, linker CH₂-piperazine), 3.43-3.35 (m, 3H, linker CH₂—Br, cyclopropane CH), 3.30-3.16 (m, 4H, piperazine 2×CH₂), 2.70-2.55 (m, 6H, piperazine 2×CH₂, linker CH₂(CH₂)₃—Br), 1.87 (quint, J=6.8 Hz, 2H, linker CH₂CH₂—Br), 1.74 (quint, J=7.5 Hz, 2H, linker CH₂(CH₂)₂—Br), 1.29-1.24 (m, 2H, cyclopropane CH₂), 1.13-1.06 (m, 2H, cyclopropane CH₂);

¹³C NMR (126 MHz, CDCl₃): δ_{C}=173.00 (d, J_{CF}=2.1 Hz, C═O), 166.22 (s, C═O), 153.34 (d, J_{CF}=248.4 Hz, cipro), 148.30 (s, C—H cipro), 144.62 (d, J_{CF}=10.6 Hz, cipro), 140.86 (s, linker Ar), 137.93 (d, J_{CF}=1.2 Hz, cipro), 135.17 (s, linker Ar), 129.34 (s, C—H linker Ar), 128.36 (s, C—H linker Ar), 122.71 (d, J_{CF}=7.2 Hz, cipro), 112.95 (d, J_{CF}=23.1 Hz, C—H cipro), 109.76 (s, cipro), 104.85 (d, J_{CF}=3.1 Hz, C—H cipro), 62.69 (s, linker CH₂-piperazine), 52.78 (s, piperazine CH₂), 51.98 (s, OCH₃), 49.92 (d, J_{CF}=4.4 Hz, piperazine CH₂), 34.64 (s, linker CH₂(CH₂)₃—Br), 34.55 (s, cyclopropane CH), 33.75 (s, linker CH₂—Br), 32.24 (s, linker CH₂CH₂—Br), 29.88 (s, linker CH₂(CH₂)₂—Br), 8.11 (s, cyclopropane CH₂).

ESI+ QTOFMS: calculated for C₂₉H₃₄BrFN₃O₃⁺ ([M+H]⁺) m/e 570.18 (100%); measured m/e 570.18.

Preparation of (1S,10R)-4-(2-(1,3-dioxoisoindolin-2-yl)ethyl)-1,4,7,10-tetraazabicyclo[8.2.2]tetradecane-11,12-dione (Compound 16): Compound 16 was assembled in two steps from cyclen using a modified procedure based on previously published procedures [Athey et al. 2002, supra; Sheng et al. 2007 supra; and Bellouard et al. J. Chem. Soc.—Perkin Trans. 1, 23, 3499-3505 (1999)].

Step 1: Cyclen (5.0 grams, 0.029 mol) was dissolved in absolute ethanol (100 mL) and diethyl oxalate (4.24 grams, 0.029 mol) was added dropwise at room temperature. TLC analysis (chloroform/methanol/methyl amine (33% methyl amine in ethanol), 80%:15%:5%) indicated complete consumption of cyclen after 48 hours. The solvent was removed under vacuum and the crude product then purified using silica chromatography using a chloroform/methanol elution system to yield mono-oxalyl-protected cyclen (6.40 grams, yield 97%) as a yellow oil.

¹H NMR (300 MHz, CDCl₃) δ_{H} 4.33-4.14 (m, 2H), 3.54-3.38 (m, 4H), 2.99-2.79 (m, 4H), 2.70-2.55 (m, 4H), 2.55-2.40 (m, 2H).

¹³C NMR (75 MHz, MeOD) δ_{C} 161.31 (s, C═O), 48.64 (s, CH₂), 48.31 (s, CH₂), 45.79 (s, CH₂), 43.77 (s, CH₂).

ESI+ QTOFMS calculated for C₁₀H₁₈N₄O₂Na ([M+Na]⁺) m/e 249.14; measured m/e 249.14.

Step 2: A stirred solution of the mono-oxalyl-protected cyclen from step 1 (6.40 grams, 0.028 mol), N-(2-bromoethyl) phthalimide (10.74 grams, 0.043 mol), and Cs₂CO₃ anhydrous (10.14 grams, 1.1 mol equivalents) in acetonitrile (185 mL) was heated at 60° C. The reaction was monitored by TLC using two systems: 1) chloroform/methanol/methyl amine (33% methyl amine in ethanol), 85%:14%:1%; 2) ethyl acetate/hexane, 30%:70%, which indicated complete consumption of starting material after 6 days. The reaction mixture was filtered, the solvent removed under vacuum, and the residue purified by silica chromatography using a chloroform/methanol elution system to yield compound 16 (2.71 grams, yield 24%).

¹H NMR (600 MHz, MeOD) δ_{H} 7.82 (dd, J=5.4 Hz, 3.0 Hz, 2H, C—H Ar), 7.71 (dd, J=5.4 Hz, 3.0 Hz, 2H, C—H Ar), 4.28-4.14 (m, 2H, cyclen), 3.99-3.88 (m, 1H, cyclen), 3.82-3.71 (m, 2H, CH₂-phthalimide), 3.55-3.48 (m, 1H, cyclen), 3.44-3.36 (m, 1H, cyclen), 3.18 (dt, J=12.6 Hz, 4.9 Hz, 1H, cyclen), 2.98-2.92 (m, 1H, cyclen), 2.91-2.80 (m, 2H, cyclen (1H), CH₂-cyclen (1H)), 2.80-2.62 (m, 5H, cyclen (4H), CH₂-cyclen (1H)), 2.62-2.54 (m, 2H, cyclen), 2.53-2.47 (m, 1H, cyclen), 2.47-2.38 (m, 1H, cyclen).

¹³C NMR (150 MHz, MeOD) δ_{C} 168.41 (s, C═O phthalimide), 161.12 (s, oxalyl C═O), 159.80 (s, oxalyl C═O), 134.20 (s, C—H Ar), 132.18 (s, Ar), 123.26 (C—H Ar), 55.99 (s, cyclen), 52.13 (s, cyclen), 51.58 (s, CH₂-cyclen), 50.87 (s, cyclen), 50.19 (s, cyclen), 48.35 (s, cyclen), 47.14 (s, cyclen), 46.87 (s, cyclen), 45.88 (s, cyclen), 35.13 (CH₂-phthalimide).

ESI+ QTOFMS calculated for C₂₀H₂₅N₅O₄Na ([M+Na]⁺) m/e 422.19; measured m/e 422.19.

Preparation of (1S,10R)-4-(2-(1,3-dioxoisoindolin-2-yl)ethyl)-1,4,7,10-tetraazabicyclo[8.2.2]tetradecane-11,12-dione (Compound 17a): Compound 17a was assembled in two steps starting from compounds 16 and 8d.

Step 1: Compound 16 (670 milligrams, 1.67 mmol) and compound 8d (0.96 gram, 1.82 mmol, 1.1 equivalents) were added to dry acetonitrile (40 mL) to form a solution. Stirring took place at room temperature under an argon atmosphere, and then DIPEA (0.4 mL, 1.2 equivalents) was added. TLC analysis (dichloromethane/methanol/25% NH₄OH aqueous solution 18:2:0.3) indicated complete consumption of 16 after 22.5 hours. The crude suspension was then filtered, washed extensively with acetonitrile and methanol, and the solvent was removed by evaporation under vacuum. The crude solid was dissolved in dichloromethane, filtered, and loaded onto a dichloromethane-packed silica column. The desired product was eluted in dichloromethane/methanol 1:24 to dichloromethane/methanol 1:6 to yield the corresponding phthalimide derivative 17a as a white solid (613 milligrams, 43.2%).

¹H NMR (600 MHz, CDCl₃) δ_{H} 8.33 (s, 1H, QH-2), 7.76-7.69 (m, 3H, phthalimide Ar, QH-5), 7.65-7.61 (m, 2H, phthalimide Ar), 7.16 (d, J=7.8 Hz, 2H, linker Ar), 7.12 (d, J_{CF}=7.1 Hz, 1H, QH-8), 7.06 (d, J=7.8 Hz, 2H, linker Ar), 4.15-4.05 (m, 2H, cyclen), 4.05-3.88 (m, 2H, cyclen), 3.75 (s, 3H, OCH₃), 3.69-3.61 (m, 1H, CH₂-phthalimide), 3.61-3.50 (m, 2H, CH₂-phthalimide (1H), cyclen-CH₂-Ph (1H)), 3.41-3.30 (m, 5H, cyclopropane CH, cyclen (2H), Ph-CH₂-piperazine (2H)), 3.28 (d, J=13.2 Hz, 1H, cyclen-CH₂-Ph (1H), 3.20-3.09 (m, 4H, piperazine), 2.88-2.76 (m, 2H, cyclen), 2.70-2.63 (m, 1H, cyclen), 2.63-2.57 (m, 1H, CH₂—CH₂-phthalimide), 2.57-2.33 (m, 10H, CH₂—CH₂-phthalimide (1H), piperazine (4H), cyclen (5H)), 2.32-2.17 (m, 2H, cyclen), 1.23-1.16 (m, 2H, cyclopropane CH₂), 1.05-0.98 (m, 2H, cyclopropane CH₂).

¹³C NMR (150 MHz, CDCl₃) δ_{C} 172.69 (s, cipro C═O), 167.90 (s, phthalimide C═O), 165.70 (s, cipro C═O), 159.58 (s, oxalyl C═O), 159.45 (s, oxalyl C═O), 153.02 (d, J_{CF}=247.2 Hz, cipro), 148.02 (s, cipro C—H), 144.30 (d, J_{CF}=10.8 Hz, cipro), 137.72 (s, cipro), 136.69 (s, linker Ar), 136.49 (s, linker Ar), 134.02 (s, C—H phthalimide Ar), 131.72 (s, phthalimide Ar), 129.61 (s, C—H linker Ar), 128.91 (s, C—H linker Ar), 122.99 (s, C—H phthalimide Ar), 122.33 (d, J_{CF}=7.0 Hz, cipro), 112.53 (d, J_{CF}=22.8 Hz, C—H cipro), 109.37 (s, cipro), 104.77 (s, cipro C—H), 62.23 (s, Ph-CH₂-piperazine), 58.59 (bs, C—N), 55.73 (bs, C—N), 55.22 (s, C—N), 52.51 (s, piperazine), 51.70 (s, C—N), 51.63 (s, OCH₃), 51.41 (s, C—N), 49.64 (d, J_{CF}=3.8 Hz, piperazine), 48.97-48.51 (m, C—N), 46.63 (bs, C—N), 46.32 (bs, C—N), 34.41 (s, cyclopropane CH), 34.25 (s, CH₂-phthalimide), 7.88 (s, cyclopropane CH₂).

ESI+ QTOFMS calculated for C₄₆H₅₁FN₈O₇ ([M+H]⁺) m/e 847.38; measured m/e 847.39.

Step 2: The phthalimide derivative from the previous step (1.16 grams, 1.36 mmol) was dissolved in dry ethanol (110 mL) and set stirring under argon atmosphere. After the system was cooled in an ice bath, K₂CO₃ (516 milligrams, 2.7 equivalents) and methyl amine (33% in ethanol, 110 mL) were added. The suspension stirred in the ice bath for 5 minutes before the system gradually reached room temperature. TLC analysis [chloroform/methanol/methyl amine (33% in ethanol) 31:4.5:0.3] indicated complete consumption of the starting material after 18 hours. The crude suspension was then filtered, washed extensively with ethanol, and the solvent was removed by evaporation under vacuum. The dry residue was dissolved in dichloromethane and then loaded on to a dichloromethane-packed silica column. The desired product was eluted in a 9:1 mixture of methanol and methyl amine (33% in ethanol) to yield a light yellow solid compound 17a (0.631 gram, 65%).

¹H NMR (600 MHz, CDCl₃) δ_{H} 9.84 (q, J=4.5 Hz, 0.5H, CH₃—NH—CO), 8.76 (s, 1H, QH-2), 7.95 (d, J_{CF}=13.4 Hz, 1H, QH-5), 7.36-7.21 (m, 4H, linker Ar), 7.19 (d, J_{CF}=7.7 Hz, 1H, QH-8), 4.54-3.86 (m, 3H, CH₂—N), 3.64-3.37 (m, 8H, Ph-CH₂-piperazine (2H), cyclopropane CH (1H), CH₂—N(5H)), 3.31-3.21 (m, 4H, piperazine), 2.95 (d, J=4.5 Hz, 3H, CH₃—NH—CO), 2.87-2.69 (m, 4H, CH₂—N), 2.69-2.54 (m, 6H, piperazine (4H), CH₂—N(2H)), 2.54-2.26 (m, 6H, CH₂—N), 1.34-1.23 (m, 2H, cyclopropane CH₂), 1.15-1.03 (m, 2H, cyclopropane CH₂).

¹³C NMR (150 MHz, CDCl₃) δ_{C} 175.47 (s, cipro C═O), 165.61 (d, J_{CF}=15.0 Hz, cipro C═O), 160.32 (s, oxalyl C═O), 160.03 (s, oxalyl C═O), 153.45 (d, J_{CF}=249.4 Hz, cipro), 146.58 (s, cipro C—H), 144.97 (d, J_{CF}=10.6 Hz, cipro), 138.53 (s, cipro), 137.06-136.74 (m, linker Ar), 129.91 (s, C—H linker Ar), 129.21 (s, C—H linker Ar), 121.77 (d, J_{CF}=5.6 Hz, cipro), 112.51 (d, J_{CF}=23.1 Hz, C—H cipro), 111.38 (d, J_{CF}=3.2 Hz, cipro), 104.78 (s, cipro C—H), 62.62 (s, Ph-CH₂-piperazine), 56.49-56.05 (m, C—N), 55.86 (bs, C—N), 53.52 (bs, C—N), 52.85 (s, piperazine), 51.95 (s, C—N), 50.03 (d, J_{CF}=4.1 Hz, piperazine), 49.29 (bs, C—N), 48.96 (bs, C—N), 46.51-46.00 (m, C—N), 39.03 (s, CH₂—NH₂), 34.72 (s, cyclopropane CH), 25.81 (d, J_{CF}=19.8 Hz, CH₃—NHCO), 8.24 (s, cyclopropane CH₂).

ESI+ QTOFMS calculated for C₃₈H₅₁FN₉O₄ ([M+H]⁺) m/e 716.40; measured m/e 716.40.

General Procedure for the Synthesis of Compounds 17b-d:

Compounds 17b-d were assembled in two steps starting from compounds 16 and 8e-g, respectively. The second step was based on a previously reported procedure [Mahboobi et al. Arch. Pharm. (Weinheim), 328 (4), 371-376 (1995)]. Step 1: To a stirred suspension of compound 16 and Cs₂CO₃ (1.2 equivalents) in anhydrous acetonitrile (35 mL/gram of 16) was added the appropriate bromide compound 8e-g (1-2 equivalents) under an argon atmosphere and stirring took place at 60° C. The reaction was monitored by TLC (chloroform/methanol/methyl amine (33% methyl amine in ethanol) 90%:9%:1%) until the conversion rate was higher than 90% (2-9 days). The crude mixture was filtered, washed extensively with acetonitrile and then the solvent was removed under vacuum. The dry residue was purified by silica chromatography using a methanol/dichloromethane elution system to yield a mixture of the corresponding phthalimide derivative and the hydrolysis decomposition product of the corresponding bromide 15, which proved to be impossible to separate in our hands.

Step 2: To a stirred solution of the crude from the step 1 in absolute ethanol (20 mL/gram of crude phthalimide derivative) were added anhydrous K₂CO₃ (2.7 equivalents) and methyl amine (20 mL/gram of crude phthalimide, 33% methyl amine in ethanol) and stirred at 0° C. using an ice bath. The suspension was stirred for 30 minutes before the system was allowed to reach room temperature. The reaction was monitored by TLC using the following system chloroform/methanol/methyl amine (33% methyl amine in ethanol) 90%:9%:1%, and indicated complete consumption of the starting material after 24-48 hours. The crude suspension was then filtered, washed extensively with ethanol, and the solvent removed by evaporation under vacuum. The residue as a yellow solid (1.67 gram) was purified by silica chromatography using a methanol/methyl amine elution system to yield pure compounds 17b-d as light yellow solids.

Compound 17b. Following the general procedure, compound 16 (2.0 gram, 5.01 mmol) and 8e (2.72 grams, 5.01 mmol), yielded 22% over two steps (0.8011 gram, 1.10 mmol).

ESI+ QTOFMS calculated for C₃₉H₅₃FN₉O₄ ([M+H]⁺) m/e 730.41; measured m/e 730.42.

Compound 17c. Following the general procedure, compound 16 (1.10 grams, 2.76 mmol) and 8f (3.06 grams, 5.51 mmol), yielded 44% over two steps (0.9075 gram, 1.22 mmol).

ESI+ QTOFMS calculated for C₄₀H₅₅FN₉O₄ ([M+H]⁺) m/e 744.4283; measured m/e 744.4406

Compound 17d. Following the general procedure, compound 16 (1.9 grams, 4.75 mmol) and 8g (5.4 grams, 9.5 mmol), yielded 22% over two steps (0.797 gram, 1.05 mmol).

ESI+ QTOFMS calculated for C₄₁H₅₇FN₉O₄⁺ ([M+H]⁺) m/e 758.45; measured ne 758.45.

General Procedure for the Synthesis of Compounds 18a-d:

To an anhydrous acetonitrile (50 mL/gram of 17) solution of the appropriate primary amine 17, were added the reagent N,N-di-Boc-1H-pyrazole-1-carboxamide (1 equivalent) and anhydrous K₂CO₃ (1.5 equivalents) and set stirring at room temperature under argon. The reaction was monitored by TLC using the following system: chloroform/methanol/methyl amine (25% solution in water) 90%:9%:1%, which indicated complete conversion after 1-2 days. The reaction mixture was filtered, washed extensively with acetonitrile and dichloromethane and then the solvent removed under vacuum. The dry residue as yellow solid was purified by silica chromatography using a methanol/dichloromethane/methyl amine elution system, to yield pure compounds 18a-d as yellow solids.

Compound 18a. Following the general procedure, compound 17a (0.631 gram, 0.88 mmol) yielded 82% (0.692 gram, 0.72 mmol);

¹H NMR (600 MHz, CDCl₃) δ_{H} 11.46 (s, 1H, Boc-guanidine NH), 9.83 (q, J=4.9 Hz, 1H, CH₃—NH—CO), 8.75 (s, 1H, QH-2), 8.28 (t, J=5.1 Hz, 1H, guanidine NH), 7.94 (d, J_{CF}=13.3 Hz, 1H, QH-5), 7.30-7.23 (m, 3H, linker Ar (2H), QH-8 (1H)), 7.23-7.16 (m, 2H, linker Ar), 4.36-4.03 (m, 4H, cyclen), 3.71 (d, J=13.4 Hz, 1H, cyclen-CH₂-Ph (1H)), 3.58-3.48 (m, 3H, Ph-CH₂-piperazine (2H), cyclen (1H)), 3.48-3.36 (m, 4H, cyclen-CH₂-Ph (1H), CH₂-guanidine (1H), cyclopropane CH (1H), cyclen (1H)), 3.36-3.28 (m, 1H, CH₂-guanidine (1H), 3.28-3.19 (m, 4H, piperazine), 2.93 (d, J=4.9 Hz, 3H, CH₃—NH—CO), 2.91-2.85 (m, 1H, cyclen), 2.85-2.76 (m, 2H, cyclen), 2.66-2.59 (m, 4H, piperazine), 2.59-2.51 (m, 3H, CH of cyclen-CH₂—CH₂-guanidine (1H), cyclen (2H)), 2.51-2.43 (m, 3H, CH of cyclen-CH₂—CH₂-guanidine (1H), cyclen (2H)), 2.40-2.28 (m, 2H, cyclen), 2.26-2.18 (m, 1H, cyclen), 1.45 (s, 9H, Boc), 1.41 (s, 9H, Boc), 1.30-1.24 (m, 2H, cyclopropane CH₂), 1.13-1.05 (m, 2H, cyclopropane CH₂);

¹³C NMR (150 MHz, CDCl₃) δ_{C} 175.41 (d, J_{CF}=2.2 Hz, cipro C═O), 165.58 (s, cipro C═O), 163.44 (s, guanidine C), 159.82 (s, oxalyl C═O), 159.66 (s, oxalyl C═O), 156.06 (s, Boc C═O), 153.41 (d, J_{CF}=249.9 Hz, cipro), 153.26 (s, Boc C═O), 146.49 (s, cipro C—H), 144.94 (d, J_{CF}=10.8 Hz, cipro), 138.47 (s, cipro), 137.26 (s, linker Ar), 136.85 (s, linker Ar), 129.71 (s, C—H linker Ar), 129.12 (s, C—H linker Ar), 121.71 (d, J_{CF}=7.2 Hz, cipro), 112.46 (d, J_{CF}=23.1 Hz, C—H cipro), 111.36 (s, cipro), 104.69 (d, J_{CF}=2.6 Hz, C—H cipro), 83.28 (s, Boc quaternary C), 79.26 (s, Boc quaternary C), 62.56 (s, Ph-CH₂-piperazine), 58.65 (s, cyclen-CH₂-Ph), 56.65 (s, C—N), 56.26 (s, C—N), 52.78 (s, piperazine), 52.55 (s, C—N), 52.28 (s, C—N), 49.98 (d, J_{CF}=3.7 Hz, piperazine), 49.74 (s, C—N), 49.11 (s, C—N), 47.44-47.03 (m, C—N), 38.57 (s, CH₂-guanidine), 34.63 (s, cyclopropane CH), 28.26 (s, Boc CH₃), 28.08 (s, Boc CH₃), 25.80 (s, CH₃—NHCO), 8.17 (s, cyclopropane CH₂).

ESI+ QTOFMS calculated for C₄₉H₆₉FN₁₁O₈⁺ ([M+H]⁺) m/e 958.53; measured m/e 958.57.

Compound 18b. Following the general procedure, compound 17b (0.8011 gram, 1.10 mmol) yielded 52% (0.570 gram, 0.573 mmol);

ESI+ QTOFMS calculated for C₅₁H₇₃FN₁₁O₈ ([M+Na]⁺) m/e 994.5393; measured m/e 994.6194.

Compound 18c. Following the general procedure, compound 17c (0.6873 gram, 0.924 mmol) yielded 59% (0.5409 gram, 0.55 mmol);

ESI+ QTOFMS calculated for C₅₁H₇₃FN₁₁O₈ ([M+H]⁺) m/e 985.5549; measured m/e 985.5650.

Compound 18d. Following the general procedure, compound 17d (0.757 gram, 0.99 mmol) yielded 78% (0.776 gram, 0.77 mmol);

ESI+ QTOFMS calculated for C₅₂H₇₅FN₁₁O₈⁺ ([M+H]⁺) m/e 1000.58; measured m/e 1000.59.

General Procedure for the Synthesis of Compounds 19-22:

Compound 18 was dissolved in an aqueous solution of 6 M HCl (60 mL/gram of 18) and stirring took place at 90° C. under argon atmosphere. The reaction was monitored by TLC using an eluent of MeOH/CH₂Cl₂/MeNH₂ (33% MeNH₂ in EtOH)/H₂O 33%:33%:23%:11%, MS and ¹H NMR, which indicated complete conversion after 7 days. The crude solution was evaporated under vacuum, washed extensively with diethyl ether to remove any residual HCl and then dried under vacuum. The dry residue was then purified using Sephadex LH20 and then lyophilized to yield pure compounds 19-22 as brown solids.

Compound 19. Following the general procedure, 18a (0.524 gram, 0.55 mmol) yielded 75% (0.376 gram, 0.413 mmol).

¹H NMR (600 MHz, CD₃OD at pH 10) δ_{H} 8.62 (s, 1H, QH-2), 7.76 (bs, 1H, QH-5), 7.48-7.15 (m, 5H, QH-8 (1H), linker Ar (4H)), 3.87-3.61 (m, 3H, cyclopropane CH (1H), R₂N—CH₂-Ph (2H)), 3.55 (s, 2H, R₂N—CH₂-Ph), 3.49-3.41 (m, 1H, CH₂N), 3.24-3.17 (m, 2H, CH₂N), 3.14-3.01 (m, 2H, CH₂N), 3.01-2.83 (m, 8H, CH₂N), 2.72-2.52 (m, 12H, CH₂N), 2.48-2.29 (m, 3H, CH₂N), 1.41-1.17 (m, 2H, cyclopropane CH₂), 1.16-0.94 (m, 2H, cyclopropane CH₂);

¹³C NMR (150 MHz, D₂O acidic) δ_{C} 175.79 (s, cipro C═O), 168.81 (s, cipro C═O), 156.66 (s, guanidine C), 153.25 (d, J_{CF}=251.0 Hz, cipro), 148.23 (s, cipro C—H), 144.07 (d, J_{CF}=9.9 Hz, cipro), 138.88 (s, cipro), 132.59 (s, C—H linker Ar), 132.27 (s, C—H linker Ar), 131.48 (s, linker Ar), 130.12 (s, linker Ar), 118.76 (d, J_{CF}=7.0 Hz, cipro), 110.68 (d, J_{CF}=23.9 Hz, cipro C—H), 106.72 (s, cipro C—H), 105.63 (s, cipro), 59.73 (s, R₂N—CH₂-Ph), 56.56 (s, R₂N—CH₂-Ph), 51.76 (s, CH₂N), 51.19 (s, CH₂N), 49.64 (s, CH₂N), 49.11 (s, CH₂N), 49.02 (s, CH₂N), 46.43 (s, CH₂N), 45.99 (s, CH₂N), 44.16 (s, CH₂N), 41.90 (s, CH₂N), 41.53 (s, CH₂N), 41.08 (s, CH₂N), 36.70 (s, CH₂-guanidine), 36.27 (s, cyclopropane CH), 7.56 (s, cyclopropane CH₂).

ESI+ QTOFMS calculated for C₃₆H₅₂FN₁₀O₃⁺ ([M+H]⁺) m/e 691.42; measured m/e 691.48.

Compound 20. Following the general procedure, 18b (0.540 gram, 0.556 mmol) yielded 38% (193.66 milligrams, 0.21 mmol);

¹H NMR (600 MHz, CD₃OD at pH 1) δ_{H} 8.84 (s, 1H, QH-2), 7.94 (d, J_{CF}=12.6 Hz, 1H, QH-5), 7.67 (d, J_{CF}=6.3 Hz, 1H, QH-8), 7.62 (s, J=7.9 Hz, 2H, linker Ar), 7.52 (s, J=7.9 Hz, 2H, linker Ar), 4.45 (s, 2H, Ph-CH₂-piperazine), 4.00-3.93 (m, 2H, NH), 3.89-3.81 (m, 2H, cyclopropane CH (1H), NH (1H)), 3.80-3.65 (m, 2H, NH), 3.64-3.44 (m, 21H, cyclen (12H), CH₂-guanidine (2H), NH (7H)), 3.44-3.36 (m, 5H, cyclen-CH₂CH₂-Ph (2H), cyclen (3H)), 3.36-3.27 (m, 5H, piperazine (4H), cyclen (1H)), 3.11-3.04 (m, 2H, cyclen-CH₂CH₂-Ph), 3.03-2.88 (m, 4H, piperazine), 2.84 (t, J=6.7 Hz, 2H, CH₂CH₂-guanidine), 1.51-1.32 (m, 2H, cyclopropane CH₂), 1.32-1.10 (m, 2H cyclopropane CH₂);

¹³C NMR (150 MHz, CD₃OD at pH 1) δ_{C} 175.62 (s, cipro C═O), 168.28 (s, cipro C═O), 157.32 (s, guanidine C), 153.82 (d, J_{CF}=251.4 Hz, cipro), 148.60 (s, C—H cipro), 144.58 (d, J_{CF}=10.7 Hz, cipro), 139.56 (s, Ar), 138.85 (s, Ar), 131.92 (s, C—H linker Ar), 129.81 (s, C—H linker Ar), 127.48 (s, linker Ar), 119.46 (s, cipro), 111.29 (d, J_{CF}=20.0 Hz, C—H cipro), 106.91 (s, C—H cipro), 106.34 (s, cipro), 59.69 (Ph-CH₂-piperazine), 55.35 (bs, cyclen), 51.24 (s, cyclen), 51.03 (s, CH₂CH₂-guanidine), 50.58 (bs, cyclen), 47.80 (s, piperazine), 46.54 (bs, cyclen), 43.05 (bs, piperazine), 42.42 (bs, cyclen), 41.34 (s, cyclen-CH₂CH₂-Ph), 41.03 (s, cyclen), 37.20 (s, CH₂-guanidine), 36.83-36.18 (m, cyclopropane CH), 28.62 (s, cyclen-CH₂CH₂-Ph), 7.35 (s, cyclopropane CH₂).

ESI+ QTOFMS calculated for C₃₇H₅₄FN₁₀O₃⁺ ([M+H]⁺) m/e 705.44; measured m/e 705.44.

Compound 21. Following the general procedure, 18c (0.450 g, 0.456 mmol) yielded 48% (0.204 gram, 0.218 mmol); ¹H NMR (600 MHz, CD₃OD at pH 10) δ_{H} 8.63 (s, 1H, QH-2), 7.88 (s, 1H, QH-5), 7.42 (s, 1H, QH-8), 7.30-7.05 (m, 4H, linker Ar), 3.67-3.56 (m, 1H, cyclopropane CH), 3.43 (s, 2H, Ph-CH₂-piperazine), 3.28-3.22 (m, 4H, CH₂N), 2.98-2.92 (m, 2H, CH₂N), 2.92-2.85 (m, 4H, CH₂N), 2.85-2.80 (m, 3H, CH₂N), 2.80-2.76 (m, 1H, CH₂N), 2.73-2.57 (m, 12H, terminal linker CH₂ (2H), CH₂N (10H)), 2.57-2.51 (m, 6H, terminal linker CH₂ (2H), CH₂N (4H)), 1.85-1.71 (m, 2H, cyclen-CH₂CH₂CH₂-Ph), 1.41-1.24 (m, 2H, cyclopropane CH₂), 1.17-1.01 (m, 2H, cyclopropane CH₂);

¹³C NMR (150 MHz, D₂O acidic) δ_{C} 175.52 (s, cipro C═O), 168.93 (s, cipro C═O), 156.72 (s, guanidine C), 153.04 (d, J_{CF}=250.1 Hz, cipro), 147.95 (s, cipro C—H), 143.86 (d, J_{CF}=10.4 Hz, cipro), 142.63 (s, Ar), 138.65 (s, Ar), 131.65 (s, C—H linker Ar), 129.32 (s, C—H linker Ar), 126.46 (s, cipro), 118.76 (s, cipro), 110.56 (d, J_{CF}=24.3 Hz, C—H cipro), 106.45 (s, C—H cipro), 60.15 (Ph-CH₂-piperazine), 52.84 (s, CH₂N), 52.35 (s, CH₂N), 50.89 (s, CH₂N), 50.17 (s, CH₂N), 49.55 (s, CH₂N), 47.98 (s, CH₂N), 46.43 (s, CH₂N), 46.35 (s, CH₂N), 44.61 (s, CH₂N), 41.94, (s, CH₂N), 41.82 (s, CH₂N), 40.80 (s, CH₂N), 36.70 (s, CH₂-guanidine), 35.97 (s, cyclopropane CH), 31.43 (s, cyclen-(CH₂)₂CH₂-Ph), 23.91 (s, cyclen-CH₂CH₂CH₂-Ph), 7.46 (s, cyclopropane CH₂).

ESI+ QTOFMS calculated for C₃₈H₅₆FN₁₀O₃⁺ ([M+H]⁺) m/e 719.45; measured m/e 719.45.

Compound 22. Following the general procedure, 18d (0.392 gram, 0.39 mmol) yielded 60% (0.221 gram, 0.23 mmol).

¹H NMR (600 MHz, CD₃OD at pH 1) δ_{H} 8.92 (s, 1H, QH-2), 7.99 (d, J_{CF}=13.0 Hz, 1H, QH-5), 7.74 (bs, 1H, QH-8), 7.61 (d, J=6.6, 2H, linker Ar), 7.41 (d, J=6.6, 2H, linker Ar), 4.49 (s, 2H, Ph-CH₂-piperazine), 4.08-4.00 (m, 2H, NH), 4.00-3.91 (m, 2H, cyclopropane CH (1H), NH (1H)), 3.87-3.76 (bs, 1H, NH), 3.70-3.59 (m, 14H, cyclen (6H), NH (8H)), 3.56-3.51 (m, 5H, CH₂-guanidine (2H), cyclen (3H)), 3.51-3.44 (m, 5H, cyclen), 3.44-3.38 (m, 6H, cyclen-CH₂(CH₂)₃-Ph (2H), piperazine (4H)), 3.29-3.21 (m, 1H, cyclen), 3.12-3.04 (m, 4H, piperazine), 2.99-2.93 (m, 1H, cyclen), 2.91 (t, J=7.6, 2H, CH₂CH₂-guanidine), 2.85-2.73 (m, 2H, cyclen-(CH₂)₃CH₂-Ph), 2.04-1.90 (m, 2H, cyclen-CH₂CH₂(CH₂)₂-Ph), 1.85-1.73 (m, 2H, cyclen-(CH₂)₂CH₂CH₂-Ph), 1.57-1.44 (m, 2H, cyclopropane CH₂), 1.38-1.22 (m, 2H, cyclopropane CH₂);

¹³C NMR (150 MHz, CD₃OD at pH 1) δ_{C} 175.17 (s, cipro C═O), 168.31 (s, cipro C═O), 158.16, 157.30 (s, guanidine C×2 tautomers), 153.87 (d, J_{CF}=250.0 Hz, cipro), 148.61 (s, cipro C—H), 144.77 (d, J_{CF}=10.9 Hz, cipro), 144.09 (s, Ar), 139.67 (s, Ar), 131.54 (s, C—H linker Ar), 129.18 (s, C—H linker Ar), 126.25 (s, linker Ar), 118.96 (s, cipro), 111.20 (d, J_{CF}=23.7 Hz, C—H cipro), 106.93 (s, cipro C—H), 106.16 (s, cipro), 59.92 (Ph-CH₂-piperazine), 54.73 (s, cyclen), 51.15 (s, CH₂CH₂-guanidine), 51.04 (s, cyclen), 50.25 (s, cyclen), 48.22 (s, piperazine), 46.63 (s, cyclen), 43.16 (s, piperazine), 42.61 (s, cyclen), 42.11 (s, cyclen), 41.31 (s, cyclen-CH₂(CH₂)₃-Ph), 40.57 (s, cyclen), 37.15 (s, CH₂-guanidine), 36.67-36.40 (m, cyclopropane CH), 34.48 (s, cyclen-(CH₂)₃CH₂-Ph), 27.77 (s, cyclen-(CH₂)₂CH₂CH₂-Ph), 22.98 (s, cyclen-CH₂CH₂(CH₂)₂-Ph), 7.47 (s, cyclopropane CH₂).

ESI+ QTOFMS calculated for C₃₉H₅₈FN₁₀O₃⁺ ([M+H]⁺) m/e 733.47; measured m/e 733.52.

The conformational flexibility, steric bulk and pKa of the pendant (general acid) warhead are crucial both for (i) proper coordination of the pendant cap with the central metal and its subsequent dynamic dissociation; and (ii) optimal leaving group activation. Therefore, an addition of primary amine-containing pendant moieties was envisioned.

FIG. 24A presents the chemical structures of exemplary ligand structures which feature a primary amine-containing moiety as an exemplary “protecting” moiety that may be coordinated in a dynamic equilibrium with the metal ion, in a reversible manner, so as to protect its poisoning by cellular and other physiological components. Such a moiety is also referred to herein as a moiety that comprises a heteroatom-containing group that has a pKa of from 6 to 8 (e.g., is non-protonated or not fully protonated at physiological pH) and is capable of reversibly binding to the metal ion (e.g., in physiological environment), wherein the heteroatom-containing group is a primary amine.

An additional set of ciprofloxacin-cyclen conjugates with amine-containing pendant moieties at ortho position was prepared accordingly.

The exemplary compounds 23-26 (FIG. 24B) represent the amine pendant analogues of compounds 19-22. These compounds were generally prepared by deprotection of the amine intermediates 17a-d from FIG. 23B, as described in FIG. 24C.

Synthesis of Ciprofloxacin-Cyclen Conjugates with Amine Pendants:

Deprotection of protected amine derivatives 17a-d (using, e.g. 6 M HCl at 90° C.) provides the desired compounds with amine pendant. The final products are characterized by various 1D and 2D NMR spectra as well as by MS.

General Procedure for the Synthesis of Compounds 23-26:

Compound 17 was dissolved in an aqueous solution of 6 M HCl (60 mL/gram of 17) and stirring took place at 90° C. under argon atmosphere. The reaction was monitored by TLC using an eluent of MeOH/CH₂Cl₂/MeNH₂ (33% MeNH₂ in EtOH)/H₂O 33%:33%:23%:11%, MS and ¹H NMR, which indicated complete conversion after 7 days. The crude solution was evaporated under vacuum, washed extensively with diethyl ether to remove any residual HCl and then dried under vacuum. The dry residue was then purified using Sephadex LH₂O and then lyophilised to yield compounds 23-26.

Compound 23. Following the general procedure, 17a (254 milligrams, 0.35 mmol) yielded 78% (0.238 gram, 0.27 mmol).

¹H NMR (600 MHz, D₂O acidic) δ_{H} 8.30 (s, 1H, QH-2), 7.76-7.44 (m, 4H, linker Ar), 7.20 (bs, 1H, QH), 7.06 (bs, 1H, QH), 4.49-4.33 (m, 4H, R₂N—CH₂-Ph×2), 3.89-3.39 (m, 6H, NH (5H), cyclopropane CH (1H)), 3.36-3.15 (m, 9H, CH₂N), 3.15-3.05 (m, 6H, CH₂N), 3.04-2.98 (m, 3H, CH₂N), 2.98-2.90 (m, 4H, CH₂N), 2.86-2.70 (m, 6H, CH₂N), 1.34-1.08 (m, 2H, cyclopropane CH₂), 1.08-0.81 (m, 2H, cyclopropane CH₂);

¹³C NMR (150 MHz, D₂O acidic) δ_{C} 175.63 (s, cipro C═O), 168.66 (s, cipro C═O), 153.20 (d, J_{CF}=250.5 Hz, cipro), 148.10 (s, cipro C—H), 144.01 (d, J_{CF}=10.1 Hz, cipro), 138.77 (s, cipro), 132.67 (s, C—H linker Ar), 132.47 (s, C—H linker Ar), 130.36 (s, linker Ar), 130.09 (s, linker Ar), 118.53 (d, J_{CF}=6.3 Hz, cipro), 110.60 (d, J_{CF}=23.0 Hz, C—H cipro), 106.59 (s, C—H cipro), 105.55 (s, cipro), 59.82 (s, R₂N—CH₂-Ph), 56.52 (s, R₂N—CH₂-Ph), 51.47 (s, CH₂N), 51.23 (bs, CH₂N), 49.43 (s, CH₂N), 48.79 (s, CH₂N), 47.93 (s, CH₂N), 46.36 (bs, CH₂N), 44.48 (s, CH₂N), 41.93 (bs, CH₂N), 40.91 (s, CH₂N), 36.22 (s, cyclopropane CH), 34.15 (s, CH₂—NH₂), 7.58 (s, cyclopropane CH₂).

ESI+ QTOFMS calculated for C₃₅H₅₀FN₈O₃⁺ ([M+H]⁺) m/e 649.40; measured m/e 649.50.

Compound 26. Following the general procedure, 17d (0.852 gram, 1.125 mmol) yielded 47% (0.484 gram, 0.53 mmol).

ESI+ QTOFMS calculated for C₃₈H₅₆FN₈O₃ ([M+H]⁺) m/e 691.4381; measured m/e 691.4395.

By using similar procedures to the ones described for compounds 1-6 in Example 1, the corresponding metal complexes of 19-23 and 26 were prepared and analyzed.

General Procedure for the Synthesis and Characterization of Aqueous Cu(H1)-Complexes:

A stoichiometric amount of Cu(ClO₄)₂ was added to an aqueous solution of the hydrochloride salt of the cyclen derivative (19-22). The pH was then adjusted to 10 using 1 M NaOH_{(aq)} and then stirring took place overnight at room temperature. UV-vis spectroscopy was then used to follow the progress of the complexation until completion. The resulting solution was pH adjusted to 7.4 to give the stock for analysis and testing. The resulting Cu(II) complexes were characterized by UV-vis, EPR, MS and potentiometric titration and the data are presented in Table 1.

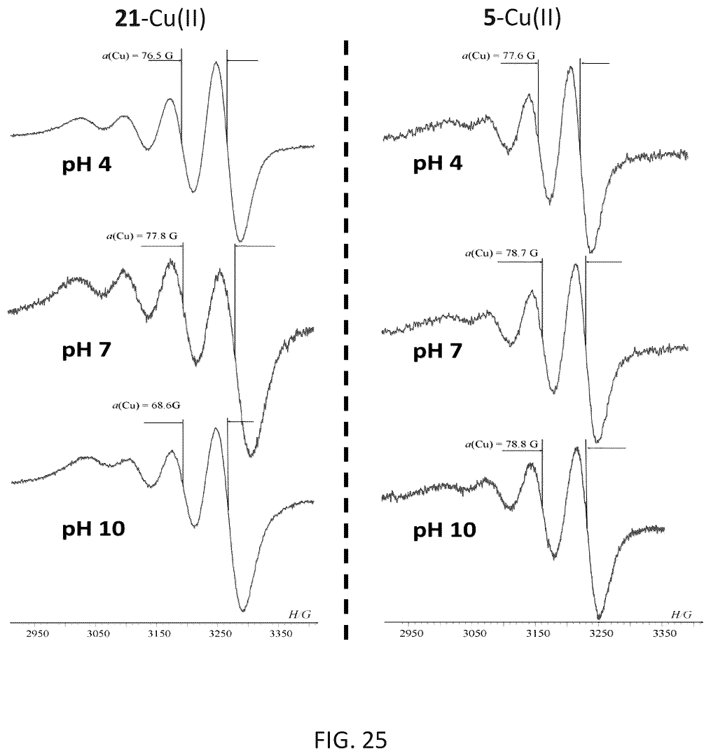

EPR of 21-Cu(II): The interaction between the unpaired d-orbital electron of 21-Cu(II) and the most abundant copper isotopes with non-zero nuclear spin (^{63,65}Cu, I=3/2), gives rise to the so called metal hyperfine interaction consisting in this particular case of four lines (2I+1), as can be seen in FIG. 25.

The hyperfine coupling constant, A, is a measure of the strength of the coupling between the electron and the nuclear spins. Only the isotropic contribution, Aᵢₛₒ, is observed in solution, which is_a measure of the s orbital character of the unpaired electron [Karunakaran, C. Spin Resonance Spectroscopy: Principles and Applications (2018)]. Hence, a smaller Aᵢₛₒ constant is indicative of diminished s-orbital character in the unpaired electron [Mabbs et al. Electron Paramagnetic Resonance of d Transition Metal Compounds. Amsterdam, Netherlands: Elsevier (1992)].

As FIG. 25 also depicts, a significantly reduced Aᵢₛₒ at pH 10 was observed (i.e. guanidine fully complexed) but not at pH 4 or 7, whilst no such effect is observed for the parent complex 5-Cu(II). These data therefore corroborate the UV-VIS and potentiometric titration data and indicate that the guanidine complexes only at high pH.

Without being bound by any particular theory, it is suggested that this phenomenon is caused by the strong π-donor character of the coordination of the guanidine to the Cu(II) center (see, for example, UV-VIS data in Table 1), since the SOMO (synonymous with the HOMO in this case) could be expected to have a diminished s orbital character when the guanidine is complexed.

General Procedure for the Synthesis and Characterization of Aqueous Zn(II)-Complexes:

A stoichiometric amount of ZnCl₂ was added to an aqueous solution of the hydrochloride salt of the cyclen derivative (19-22). The pH was then adjusted to 10 using 1M NaOH_{(aq)} and then stirring took place for 24 hours at room temperature. Mass spectrometry was used to confirm that the Zn(II) complexes had been successfully prepared. The resulting solution was pH adjusted to 7.4 to give the stock for analysis and testing. The resulting Zn(II) complexes were characterized by MS, ¹³C NMR, and potentiometric titration and the data are presented in Table 1.

NMR of 22-Zn(II) (not shown): The 22-Zn(II) complex was characterized qualitatively using ¹³C NMR at pH 8.8, which showed a strong downfield shift in the C—N region and for a part of the guanidine carbon peak (6 of about 157 ppm). These data therefore corroborate the potentiometric titration data and indicate that the guanidine coordinates to the Zn(II) via a seven-membered cycle.

General procedure for the synthesis and characterization of aqueous Co(III)-complexes of 19-22: The preparation of Co(III) complexes of 19-22 was done in a two-step process based on a previously reported procedure [Jorge et al. Chemistry, 22 (11), 3764 (2016)] with some modifications as follows.

Step 1: Synthesis of Aqua-Hydroxo Complex of 19-22

The hydrochloride salts of 19-22 were dissolved in a MeOH/H₂O mixture (1:1, 2 mL) and an equimolar amount of Na₃[Co(CO₃)₃]·3H₂O was added. The dark green solution was left to react for 16 hours at 65° C. The solution was filtered while hot under gravity to separate the liquid from a black solid. The filtrate was dried under vacuum and re-dissolved in methanol (4 mL), and the resulting solution was filtered to remove a white precipitate. The resulting filtrate was then dried under vacuum.

Step 2: Activation to the Aqua Complex of 19-22 (i.e. Guanidine Coordinated)

The compound from step 1 was re-dissolved in water and then pH adjusted to 10. An immediate bathochromic shift in the d-d transition band indicated that the guanidine had become coordinated to the Co(III) center. The resulting solution was pH adjusted to 7.4 to give the stock for analysis and testing. The complexes were further characterized by MS, ¹³C NMR and potentiometric titration and the data are presented in Table 1.

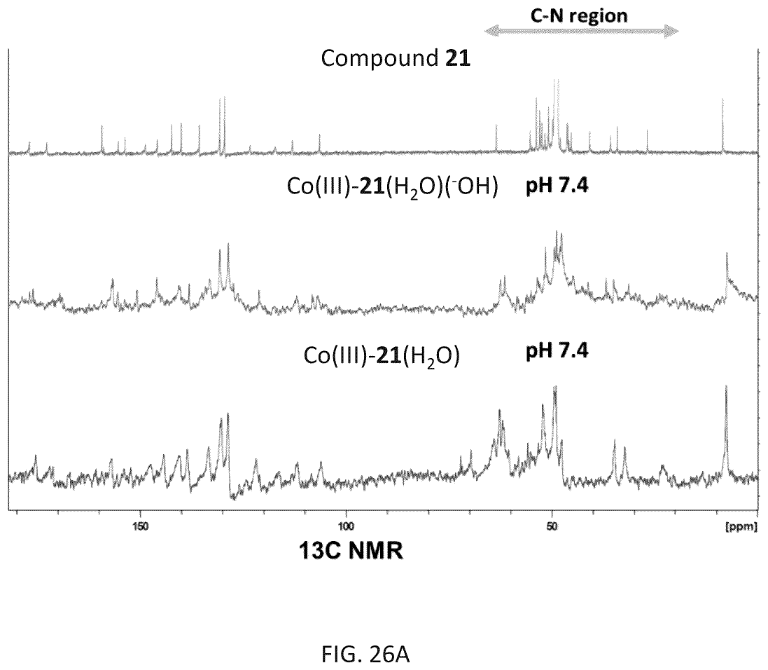

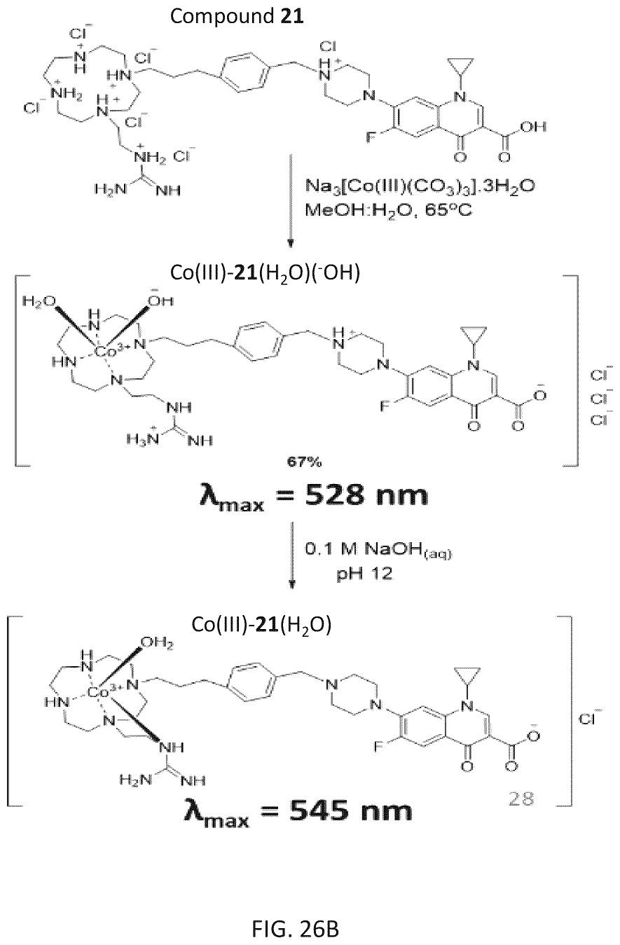

NMR of 21-Co(III) (FIG. 26A): The aqua-hydroxo complex [Co(III)-21(H₂O)(—OH)] was characterized qualitatively by ¹³C NMR, which showed a strong downfield shift in the C—N region due to Co(III) complexation as expected. Upon pH adjustment of the aqua-hydroxo complex [Co(III)-21(H₂O)(—OH)] from 7.4 to 10 and then back to 7.4, a small but significant bathochromic shift (i.e. red shift) was observed (from 528 nm to 545 nm). Furthermore, a strong downfield shift was observed for two ‘C—N region’ carbons in the ¹³C NMR.

These data suggest that pH adjustment from 7.4 to 10 “activates” the guanidine to coordinate to the Co(III) center. Furthermore, the fact that the additional downfield shift is observed in the C—N region and not for the guanidine carbon (δ=157 ppm), suggests that the Co(III) coordinates to the guanidine via a five-membered cycle (see complex Co(III)-21(H₂O) in FIG. 26B) and not via a seven-membered cycle.

General procedure for the synthesis and characterization of aqueous Co(III)-complexes of 23 and 26: The preparation of Co(III) complexes of the amine pendant 23 and 26 was done in a two-step process with a slightly different procedure.

The hydrochloride chloride of the cyclen derivative (23 or 26) was dissolved in a methanol/water mixture (1:1, 2 mL) and an equimolar amount of Na₃[Co(CO₃)₃]·3H₂O was added. The dark green solution was left to react for 16 hours at 65° C. The solution was filtered while hot under gravity to separate the liquid from a black solid. The filtrate was dried under vacuum and re-dissolved in methanol (4 mL), and the resulting solution was filtered to remove a white precipitate. The formation of the corresponding complex was monitored spectrophotometrically, as the amine pendant became directly coordinated with the Co(TII) center. A strong hypsochromic shift in the d-d transition band (i.e. blue shift) compared to that of the parent complexes was observed, which indicated that the second water ligand had been replaced by the stronger σ-donor amine as in accordance with the spectrochemical series [see Tsuchida Ryutaro. Absorption Spectra of Co-Ordination Compounds. 1., 13 (5), 388-400 (2006); Spin Resonance Spectroscopy, Principles and Applications, 169-228 (2018); and Mabbs et al. 1992 supra]. The resulting solution was pH adjusted to 7.4 to provide the complexes for analysis and testing. The resulting complexes were further characterized by MS analysis and potentiometric titration and the data are presented in Table 1.

Unlike the Co(III)-guanidinium complexes of 19-22, the “activation” of the pendant was not necessary for the Co(III) complexes of 23 and 26, presumably because the pendant amine is less basic than the guanidine pendant.

The analytical data of all the metal complexes (Cu(II), Zn(II) and Co(III)) are summarized in Table 1 below. NA=Not available (NA) data NC=no coordination.

TABLE 1

| | | | | | | MS |
|---|---|---|---|---|---|---|
| | δₘₐₓ | δₘₐₓ | δₘₐₓ | Pendant- | | M⁺ calc. m/e; |
| Compound | pH = 6 | pH = 8 | pH = 10 | Metal pKₐ | Yield | measured m/e |
| 19-Cu(II) | 633 | 642 | 669 | 8.6 | Quant. | 752.3342; 752.3346 |
| 19-Co(III) | 556 | 560 | 560 | <4 | 79% | 748.34; 748.37 |
| 19-Zn(II) | NA | NA | NA | 7.3 | Quant. | 753.3337; 753.3331 |
| 20-Cu(II) | 623 | 648 | 666 | 8.3 | Quant. | 766.3498; 766.3513 |
| 20-Co(III) | 540 | 538 | 536 | <4 | 87% | 762.35; 762.39 |
| 20-Zn(II) | NA | NA | NA | about 7 | Quant. | 767.3494; 767.3470 |
| 21-Cu(II) | 618 | 630 | 660 | 8.8 | Quant. | 780.3655; 780.3660 |
| 21-Co(III) | 542 | 547 | 548 | <4 | 62% | 776.37; 776.37 |
| 21-Zn(II) | NA | NA | NA | 7.8 | Quant. | 781.3650; 781.3657 |
| 22-Cu(II) | 617 | 628 | 661 | about 8.8 | Quant. | 794.3811; 794.3816 |
| 22-Co(III) | 533 | 535 | 537 | <4 | 89% | 790.38; 790.45 |
| 22-Zn(II) | NA | NA | NA | about 7.8 | Quant. | 795.3807; 795.3811 |
| 23-Cu(II) | 631 | 630 | 626 | NC | Quant. | 710.3124; 710.3131 |
| 23-Co(III) | 501 | 499 | 498 | <4 | 81% | 706.32; 706.32 |
| 23-Zn(II) | NA | NA | NA | NC | Quant. | 711.3119; 711.3124 |
| 26-Cu(II) | 620 | 619 | 621 | NC | Quant. | 752.3593; 752.3599 |
| 26-Co(III) | 496 | 499 | 499 | <4 | 60% | 748.36; 748.36 |
| 26-Zn(II) | NA | NA | NA | NC | Quant. | 753.3589; 753.3599 |

### Example 6

### DNA Cleavage Assays of Pendant-Bearing Complexes

To evaluate the nuclease activity of the exemplary compounds 19-23 and 26 alone and as their metal complexes with Cu(II), Co(III), and Zn(II), assays were performed as described in Example 2 hereinabove, in the absence of adjuvants. Agarose gel electrophoresis (1% agarose) with ethidium bromide staining was used to monitor the conversion of supercoiled (Form I) pHOT-1 plasmid DNA into its nicked form (form II); no linear form (form III) was observed. The copper complex of compound 2 (2-Cu(II)) was used as the positive control in these assays.

Cleavage Experiments in Metal-Free Ligands:

FIGS. 27A-B show that three of the metal-free ligands 19, 22 and 23, exhibit significant DNase activity at physiological pH (see also the comparative FIG. 27C). This suggests that the guanidine and amine pendant moieties are able to cooperate catalytically with the amines of the cyclen, and presumably to provide both efficient phosphate oxygen(s) stabilization and leaving group activation.

Cleavage Experiments in Cu(II) and Zn(II) Complexes:

19-Cu(II) and 23-Cu(II) show significant hydrolytic DNase activity (see FIGS. 27A-B) that is comparable to that of the respective complex 4-Cu(II). The new Cu(II) complexes (19-23 and 26) exhibited similar activity to that of ligands 4-6. 20-Cu(II) did not exhibit significant DNase activity, unlike its parent complex 5-Cu(II).

These data suggest that the two-carbon linked guanidine or amine pendant moieties may not be sufficient for orientating appropriately for cooperative hydrolytic catalysis in the new Cu(II) complexes. The Zn(II) complexes did not exhibit a significant DNase activity (see FIGS. 27A-B). Since the Cu(II) and Zn(II) complexes share the same geometry and also the same conformation about the cyclen ring [see: Zhang et al. Organometallics, 33, 8, 1925-1935 (2014); and Ichimaru et al. IUCrData, 6 (4), x210397 (2021)], it means that the steric interactions of the guanidine or amine pendant with DNA should be similar for both metals.

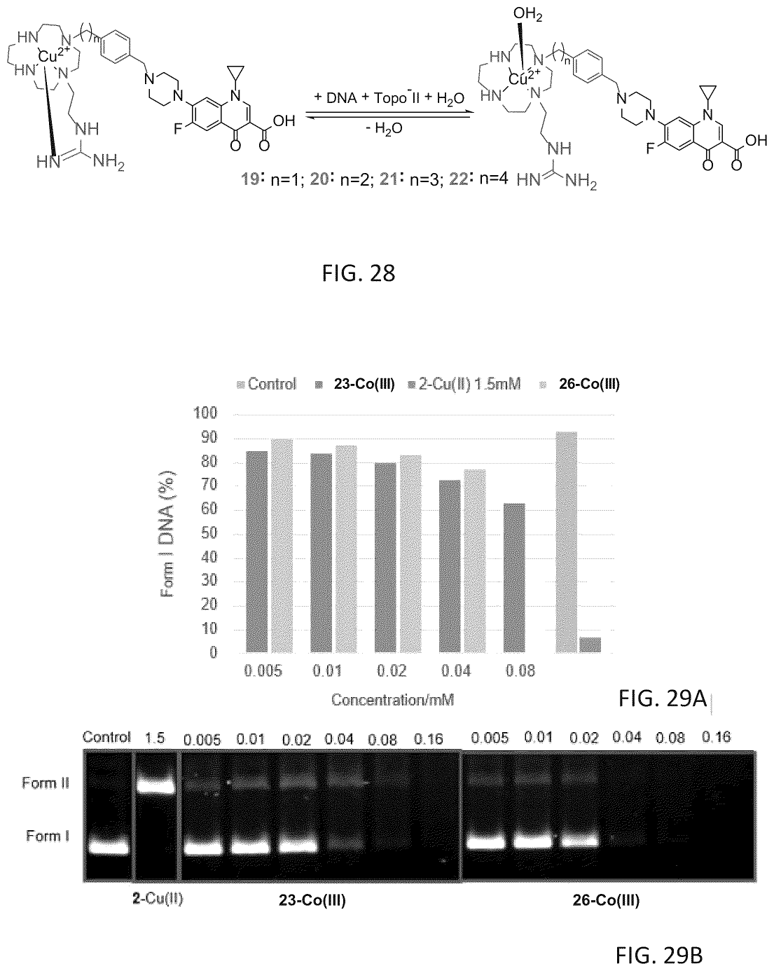

Without being bound by any particular theory, it has been assumed that at physiological pH, the pendant groups are mostly coordinated with metal ions (as shown schematically for compounds 19-22 in FIG. 28), but when the ligands approach the phosphodiester bond inside the ternary complex, the equilibrium favors the open species due to the strong electrostatic interactions between the metal/pendant and the phosphodiester of the DNA [see: Tjioe et al. 2012 supra; and Belousoff et al. Inorg. Chem., 47, 19, 8641-8651 (2008)].

The measured pKa values of the Cu(II) complexes of 19-22 (Table 1) are in the range of 8.3-8.8, suggesting that at pH 7.4 in which the DNA cleavage experiments were performed, all the complexes would be mostly in their open-conformation. The pKa values of the 19-22-Zn(II) complexes (Table 1) are lower yet close to physiological pH, suggesting that a large proportion of theses complexes would also be in the open-conformation.

These complexes are further tested in the presence of various endogenous additives, such as primary amine, glutamate and ATP.

Cleavage Experiments in Co(III) Complexes:

Although the pKₐs of the Co(III) complexes with pendant amine/guanidine of 19-23 and 26 (Table 1) are significantly below physiological pH (pKₐs<4), it was envisaged that DNA would induce the opening of the metal-pendant complex as has been previously reported for Cu(II) complexes that possess a similarly low pendant pKa [see: Tjioe et al. 2012 supra; and Belousoff et al. 2008 supra).

The fact that the DNA could not be recovered at the higher concentrations tested (see, FIGS. 27A, 29A and 29B) suggests that the pendant moiety is mostly uncoordinated to the metal in the presence of DNA. It should be noted that for 20-Co(III) (FIG. 27A), most of the DNA is recovered at all concentrations, suggesting that this complex remains in the closed state even in the presence of DNA.

23-Co(III) and 26-Co(III) complexes exhibit significant DNase activity at micromolar concentrations (see, FIGS. 27A and 27B) and in a dose-dependent fashion, suggesting that at the very least, the amine pendant moiety does not interfere with the hydrolytic activity of the Co(III)-cyclen moiety, and at best it enhances its activity. Since the Co(III) complexes have a geometry (trigonal bipyramidal) and conformation about the ring (syn-anti) [Zhang et al. 2014, supra] different from the Cu(II) and Zn(II) complexes, it is reasonable to expect that the steric interactions with DNA would be different.

The corresponding guanidine-pendant 19-Co(III) and 22-Co(III) complexes (FIG. 27A) showed less significant DNase activity. Evidently, the more bulky guanidine-containing pendant moiety sterically hinders the approach of the catalytic warhead to the DNA even for the Co(III) complex.

Overall, the Co(III) complexes bearing amine-pendant exhibited the best DNA-cleaving performances, which is indicative of their antibacterial activity.

### Example 7

### Antibacterial Activity

Compounds 1-6 and their Cu(II) and Co(III) complexes, as well as the pendant-bearing compounds 19-23 and 26 were tested for their comparative antibacterial activity by determining MIC values against two Gram-negative E. coli strains (AR477-100 and 25922) and two Gram-positive strains, Staphylococcus Epidermidis, and Bacillus Subtilis. The observed data is presented in Table 2 below and show that all the compounds exhibit significant antibacterial activity against Gram-negative and Gram-positive bacteria, with compounds 5 and 6 (with an aromatic linker) and their Co(III) complexes, 5-Co(III) and 6-Co(III), along with the pendant-bearing Compound 22 and complexes 22-Co(III), 23-Co(III) and 26-Co(III) show slightly higher activity in comparison to the rest of the compounds against all the tested bacteria.

The metal-free ligands 1-6, however, exhibit reduced antibacterial activity relative to the parent ciprofloxacin.

All the Cu(II) and Co(III) complexes of 1-6 exhibit similar antibacterial activity compared to the corresponding compounds without the metal ion, corroborating the IC₅₀ data for the Cu(II) complexes (Table 2) and the DNA cleavage assay data for Co(III) complexes (FIG. 8). However, when the MIC was determined in the presence of a high concentration of DTT (10 mM), 4-Cu(II) showed significantly improved antibacterial activity (8-fold) against E. coli R477-100 (MIC values of 3.0 and 24 μg/mL respectively with and without DTT) whilst CuCl₂ and 1-Cu(II) showed no change whatsoever under the same conditions. A similar trend (4-fold improvement) was observed for 4-Cu(II) against another E. coli strain (25922) and against another bacterial species (B. Subtilis). These data corroborate the findings of the DNA gyrase cleavage assay (see, FIGS. 12A-B) and provide an ex-vivo proof of concept. Presumably, the high concentration of DTT is able to compete effectively with endogenous Cu(II)-cyclen poisons such as ATP and glutamate, thereby facilitating the oxidative DNase activity of 4-Cu(II) even under ‘vulnerabilizing’ cellular conditions.

Compounds 19-23, 26 and their Cu(II), Zn(II) and Co(III) complexes were also tested for their comparative antibacterial activity in similar assays. The data are presented in Table 2 below and indicate all pendant-bearing derivatives of ciprofloxacin exhibit antibacterial activity against Gram-negative and Gram-positive bacteria.

[a] Escherichia coli R477-100; [b] Escherichia coli 25922; [c] Staphylococcus Epidermidis; [d] Bacillus Subtilis.

TABLE 2

| Tested | Gram-negative | | Gram-positive | | |
|---|---|---|---|---|---|
| Compound/ | MIC (μg/mL) | | MIC (μg/mL) | | IC₅₀ |
| Complex | a | b | c | d | (μM) |
| Cipro | 0.05 | 0.05 | 0.19 | 0.02 | 0.11 ± 0.01 |
| 1 | 12 | 24 | 12 | 12 | 0.13 ± 0.03 |
| 1-Cu(II) | 24 | 48 | 24 | 48 | 0.10 ± 0.01 |
| 1-Co(III) | 12 | 12 | 24 | 12 | — |
| 2 | 12 | 6 | 12 | 12 | 0.24 ± 0.03 |
| 2-Cu(II) | 12 | 6 | 12 | 12 | 0.24 ± 0.03 |
| 2-Co(III) | 12 | 6 | 24 | 12-24 | — |
| 3 | 24-48 | 12 | 6 | 6 | — |
| 3-Cu(II) | 12 | 6 | 6 | 6 | — |
| 3-Co(III) | 12 | 6 | 12-24 | 12 | — |
| 4 | 12-24 | 12-24 | 6-12 | 6-12 | 0.15 ± 0.01 |
| 4-Cu(II) | 24 | 12 | 3 | 6 | 0.14 ± 0.02 |
| 4-Co(III) | 12 | 6 | 6 | 6 | — |
| 5 | 6 | 3 | 3 | 3 | 0.15 ± 0.04 |
| 5-Cu(II) | 6 | 6 | 6 | 3 | 0.18 ± 0.03 |
| 5-Co(III) | 3 | 1.5 | 3 | 3 | — |
| 6 | 3 | 1.5 | 1.5 | 3 | 0.07 ± 0.02 |
| 6-Cu(II) | 6 | 3 | 3 | 6 | 0.09 ± 0.01 |
| 6-Co(III) | 6 | 3 | 3 | 3 | — |
| CuCl₂ | >384 | >384 | >384 | >384 | — |
| 19 | 12 | 12 | 6 | 6 | — |
| 19-Cu(II) | 12 | 12 | 12 | 6 | — |
| 19-Co(III) | 12 | 6 | 6 | 6 | — |
| 19-Zn((II) | 12 | 6 | 3 | 3 | — |
| 20 | 24 | 24 | 24 | 12 | — |
| 20-Cu(II) | 12 | 12 | 24 | 6 | — |
| 20-Co(III) | 6 | 6 | 12 | 12 | — |
| 20- Zn(II) | 12 | 6 | 12 | 6 | — |
| 21 | 12 | 12 | 6 | 6 | — |
| 21-Cu(II) | 48 | 48 | 24 | 12 | — |
| 21-Co(III) | 24 | 12 | 24 | 12 | — |
| 21-Zn(II) | 24 | 6 | 24 | 6 | — |
| 22 | 3 | 3 | 6 | 1.5 | — |
| 22-Cu(II) | 6 | 6 | 12 | 6 | — |
| 22-Co(III) | 6 | 3 | 6 | 1.5 | — |
| 22-Zn(II) | 12 | 6 | 12 | 3 | — |
| 23 | 24 | 24 | 12 | 6 | — |
| 23-Cu(II) | 24 | 24 | 12 | 12 | — |
| 23-Co(III) | 6 | 3 | 3 | 3 | — |
| 23-Zn(II) | 24 | 24 | 12 | 12 | — |
| 26 | 12 | 12 | 6 | 6 | — |
| 26-Cu(II) | 6 | 6 | 6 | 3 | — |
| 26-Co(III) | 3 | 3 | 6 | 1.5 | — |
| 26-Zn(II) | 6 | 6 | 6 | 6 | — |

Compound 22 exhibits significantly better antibacterial activity (4-8 fold) compared to all the other pendant-bearing metal-free ligands. These data correlate to the significant DNase activity exhibited by the metal-free ligands 19, 22 and 23 at physiological pH. Given the structural similarity between the ligands, it is unlikely that this difference can be attributed to pharmacokinetic differences between 22 and the other ligands. These data therefore suggest that compound 22 might be operating via a catalytic mechanism in the absence of any adjuvants from within the ternary complex, similarly to 2t-Cu(II) and 4-Cu(II) in the presence of DTT.

The MIC of 19-Cu(II) is similar to that of the metal-free ligand. Although 19-Cu(II) exhibits significant DNase activity, since the pKa of its cap (8.8) is far above physiological pH, the Cu(II) center would be largely unprotected from endogenous chelators within a cellular environment. 22-Cu(II) also shows similar MIC value as the metal-free ligand, which correlate to UV-VIS spectroscopy and potentiometric titration data which indicated that there is no coordination between the pendant amine and the Cu(II) center (see, Table 2). Other Cu(II) and all Zn(II) complexes show no significant DNase activity, therefore no improvement in MIC was expected for these complexes.

23-Co(III) and 26-Co(III) show significantly improved MICs in both Gram-negative and Gram-positive bacteria, which corroborate the former data since both complexes i) exhibit significant DNase activity (see FIG. 29B) and (ii) are capped by the pendant amine at physiological pH (pKₐ<4). More specifically, 23-Co(III) shows a 4-8 fold improvement in Gram-negative bacteria and 2-4 fold improvement in Gram-positive bacteria, whilst 26-Co(III) shows a 4-fold improvement in Gram-negative bacteria and a 2-fold improvement in Gram-positive bacteria (see Table 2). These data therefore suggest that these two complexes might be operating via a metal-derived catalytic mechanism from within the ternary complex, under physiological conditions, similarly to 2-Cu(II) and 4-Cu(II) in the presence of DTT.

20-Co(III), although exhibiting low DNase activity and maintaining coordination in the presence of DNA, showed a significantly improved MIC in both Gram-negative (4-fold) and Gram-positive (2-fold) bacteria. These data suggest that 20-Co(III) might also operate catalytically within the ternary complex. Without being bound by any particular theory, it is assumed that the unusually stretched-DNA topology within the ternary complex [Bax et al. Journal of Molecular Biology. Academic Press, 3427-3449 (2019)] might facilitate a less sterically congested meeting between the DNA and the 20-Co(III), which lowers the kinetic barrier for opening the cap (i.e. via phosphate oxygen coordination to the Co(III)).

In additional assays, the MIC of the pendant-bearing ciprofloxacin-cyclen derivatives was determined in the presence of low and high concentrations of DTT (2 and 10 mM, respectively).

The observed data are presented in Table 3 below.

[a] Escherichia coli R477-100; [b] Bacillus Subtilis.

TABLE 3

| Compound/ | | | | | | |
|---|---|---|---|---|---|---|
| Complex | Gram-negative MIC (μg/mL)^{a} | | | Gram-positive MIC (μg/mL)^{b} | | |
| DTT | 0 mM | 2 mM | 10 mM | 0 mM | 2 mM | 10 mM |
| Cipro | 0.05 | 0.05 | 0.05 | 0.02 | 0.02 | 0.02 |
| 19 | 12 | 12 | 12 | 6 | 6-12 | 6 |
| 19-Cu(II) | 12 | 6 | 3 | 6-12 | 6 | 3 |
| 19-Co(III) | 6 | 3 | 1.5 | 3 | 0.75 | 0.375 |
| 19-Zn((II) | 12 | 12 | 6 | 6 | 6-12 | 6 |
| 20 | 24 | 12 | 24 | 12 | 6-12 | 12 |
| 20-Cu(II) | 12 | 6-12 | 6 | 6 | 3 | 1.5 |
| 20-Co(III) | 12 | 6 | 3 | 6 | 3 | 1.5 |
| 20- Zn(II) | 12 | 12 | 6 | 12 | 6-12 | 12 |
| 21 | 12 | 6 | 6 | 12 | 12 | 6 |
| 21-Cu(II) | 24 | 6 | 6 | 24 | 6 | 3 |
| 21-Co(III) | 24 | 12 | 1.5 | 12 | 6 | 1.5 |
| 21-Zn(II) | 24 | 12 | 12 | 12 | 6 | 12 |
| 22 | 3 | 3 | 3 | 1.5 | 1.5 | 1.5 |
| 22-Cu(II) | 6 | 3 | 0.75 | 6 | 3 | 1.5 |
| 22-Co(III) | 6 | 3 | 1.5 | 1.5 | 0.75 | 0.375 |
| 22-Zn(II) | 6 | 3 | 3 | 3 | 1.5 | 1.5 |
| 23 | 24 | 12 | 12 | 6 | 6 | 3 |
| 23-Cu(II) | 24 | 6 | 3 | 12 | 6 | 3 |
| 23-Co(III) | 6 | 3 | 0.75 | 3 | 1.5 | 0.75 |
| 23-Zn(II) | 24 | 12 | 12 | 12 | 6 | 12 |
| 26 | 12 | 6 | 12 | 6 | 6 | 6 |
| 26-Cu(II) | 6 | 3 | 3 | 3 | 3 | 1.5 |
| 26-Co(III) | 3 | 3 | 1.5 | 1.5 | 0.75 | 0.75 |
| 26-Zn(II) | 6 | 3 | 3 | 6 | 3 | 3 |

The addition of DTT did not affect the MIC of the metal-free compounds 19-23 and 26 or their Zn(II) complexes, while both the Cu(II) and Co(III) complexes showed significant improvements in the measured MIC values. The compound 21-Co(III) showed the greatest improvement in the presence of 10 mM DDT; 16-fold improvement in Gram-negative and 8-fold improvement in Gram-positive bacteria. The complexes 22-Cu(II), 23-Cu(II) and 22-Co(III) also exhibited significant improvement in antibacterial activity, which was observed mainly against the Gram-negative bacteria.

The compound 23-Co(III) exhibited 4-8-fold better activity in Gram-negative E. coli than the parent drug without metal (compound 23, Table 2) and further 8-fold improvement in the presence of 10 mM DTT (Table 3). Against E. coli R477-100 it showed 32-fold improvement in the MIC in the presence of 10 mM DTT [MIC (23)=24 mg/mL vs. MIC 23-Co(III)=0.75 mg/mL].

Overall, all tested compounds exhibited antibacterial activity. The Co(III) complexes bearing amine-pendant corroborate the prediction from Example 6 by exhibiting the best performances with or without DTT, especially in the presence of Gram-negative pathogenic bacteria.

### Example 8

### Vulnerability of Co(III) Complex Under Physiological Conditions

In order to examine the vulnerability of a second-generation complex at physiological conditions in the presence of endogenous chelators, 23-Co(III) was further tested: (i) in TRIS buffer (instead of HEPES buffer which was the standard buffer in all the experiments); (ii) in TRIS buffer in the presence of glutamate and (iii) in TRIS buffer in the presence of ATP, with Co(III)-cyclen as control (see, FIGS. 31A and 31B). The data shows that the complex 23-Co(III) retains full DNase activity in TRIS buffer (meaning that it is devoid of the vulnerability in the presence of primary amine) and TRIS buffer in the presence of 100 mM glutamate, and partial activity in the presence of 1.8 mM ATP in Tris buffer. In contrast, Co(III)-cyclen is not inhibited by ATP but is very strongly inhibited by glutamate.

These data demonstrate that the amine cap is capable of cleaving DNA catalytically, while protecting the metal center from poisoning by endogenous chelators like primary amine, glutamate and ATP.

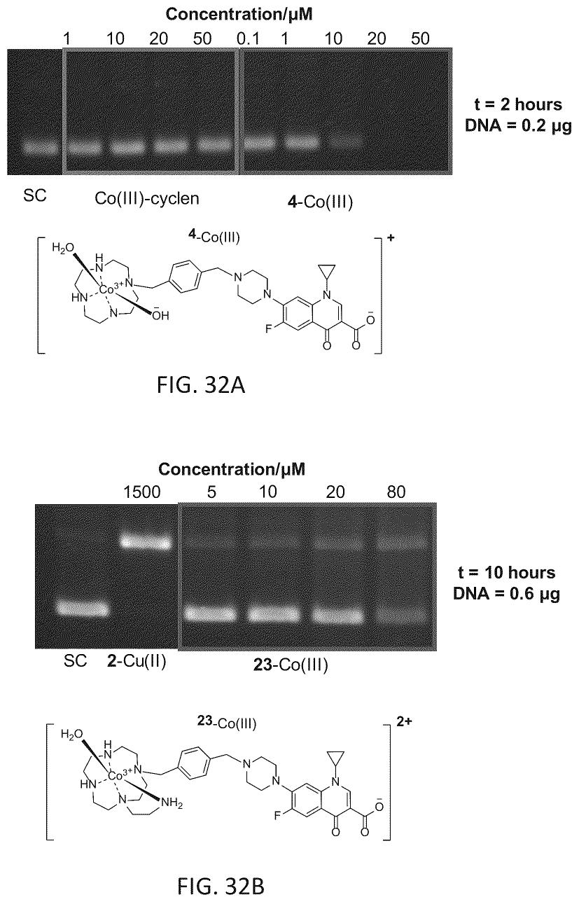

DNase activity was then examined as described hereinabove. As shown in FIG. 32A, Co(III)-cyclen is inactive at micromolar concentrations (1-50 μM), whilst 4-Co(III) binds strongly to DNA, while showing no DNase activity. In FIG. 32B, however, 23-Co(III) shows strong DNase activity at micromolar concentrations.

These data demonstrate that the dynamic amine pendant cap facilitates catalysis by inducing detachment of the metal center from DNA and by general acid-catalysis in the presence of the amine/ammonium pendant.

### Example 9

### Concluding Insights

Metal-cyclen complexes (Cu(II)-cyclen and Co(III)-cyclen) were chosen as potential catalytic warheads, covalently attached to a ciprofloxacin scaffold through different lengths of aliphatic (compounds 1-3) and aromatic (compounds 4-6) linkers.

The obtained data show that the in vitro DNase activity of the Cu(II) complexes of ligands 1-6 in the absence of type IIA topoisomerase enzymes was concentration-dependent and significantly higher than that of the Cu(II)-cyclen itself, both under hydrolytic and oxidative conditions. The observed hydrolytic activity correlated strongly with linker length, while the oxidative activity varied. The observed data also demonstrated that the Co(III) complexes of ligands 1-6 are more active than the corresponding Cu(II) complexes. However, the Co(III) complexes of 1-6 showed exceedingly enhanced binding to plasmid DNA, suggesting that these complexes would be unable to generate a catalytic turnover.

Compounds 1-6 and their Cu(II) complexes showed good antibacterial activity against both Gram-negative and Gram-positive bacteria and proved to be highly potent bacterial DNA gyrase inhibitors, exhibiting very similar inhibition potencies for both the metal-free and Cu(II) complexes of ligands 1-6 in comparison to that of the parent ciprofloxacin (Table 2). DNA gyrase cleavage assays showed that both the metal-free and Cu(II) complexes of the ligands inhibit these enzymes by stabilizing the ternary complex like the parent ciprofloxacin. These observations demonstrate that the binding mode and the subsequent antibacterial mechanism of action of the parent ciprofloxacin scaffold are retained within the new structures 1-6 and their Cu(II) complexes.

2-Cu(II) and 4-Cu(II) were shown to generate linear DNA under oxidative conditions (with DTT), in the presence of DNA gyrase without the addition of Proteinase K, and 4-Cu(II) demonstrated significantly enhanced antibacterial activity in the presence of a high concentration of DTT.

Due to the ‘vulnerability’ of the Cu(II)-cyclen system, a second generation of compounds bearing guanidine (19-22) and amine (23 and 26) pendants and their Cu(II), Zn(II) and Co(III) complexes were prepared and evaluated in vitro for their ability to cleave plasmid DNA (in the absence of the gyrase enzyme) and ex vivo for their antibacterial activity in the presence or absence of DTT.

It was found that in vitro DNase activity of metal-free ligands 19, 22 and 23 show significant DNase activity at physiological pH, while compound 22 exhibits significantly improved antibacterial activity (4-8 fold) than all the other second-generation metal-free ligands. These data suggest that the cyclen and the guanidine parts may act in synergy via general acid/general base catalysis to cleave the DNA phosphodiester bond, in a similar manner to that previously reported for the staphylococcal nuclease enzyme [Weber et al. Biochemistry, 30 (25), 6103-6114 (1991)].

Further tuning of the linker length and the position of the guanidine pendant, as shown in FIG. 18, can provide further improvement in catalytic power and the subsequent antibacterial activity of such metal-free ligands that might be very economical and could avoid any problems of vulnerability of metal-based warheads.

Among all the tested compounds (1-6, 19-23 and 26), complex 23-Co(III) exhibits best performances. It showed significant hydrolytic DNase activity by cleaving the plasmid DNA at physiological pH, suggesting that while in solution the amine pendant should protect it from its vulnerability (pKₐ<4), in the presence of DNA the pendant is mostly uncoordinated to the metal. This unprecedented DNase activity of 23-Co(III) is further supported by its significantly improved antibacterial activity; 4-8-fold better activity in Gram-negative E. coli than the metal-free parent drug (23). Furthermore, 23-Co(III) displayed additional 8-fold improvement in the presence of 10 mM DTT, with an overall 32-fold improvement in activity against E. coli R477-100.

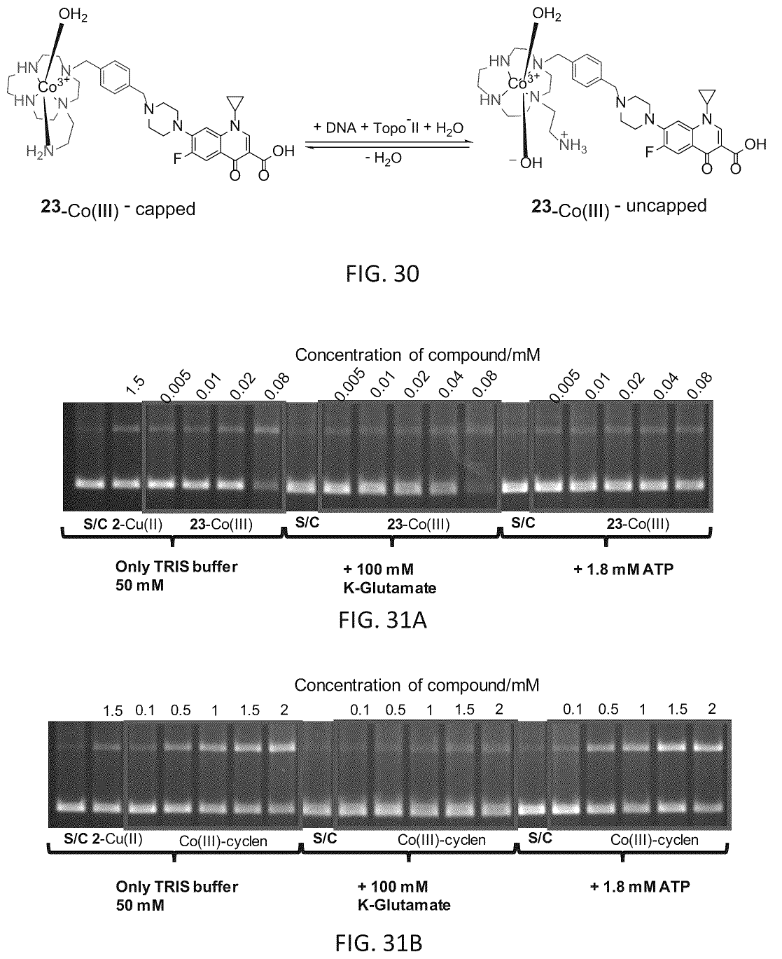

It is to be noted that the Co(III)-amine cap equilibrium present in 23-Co(III) (see, FIG. 30) also facilitates the dissociation of the Co(III) metal center from the phosphate oxygen/s post DNA cleavage. In the post-cleavage state, the pendant is in its free-amine form and therefore available for coordination at the Co(III) center. Thus, the amine cap is critical not only for protecting the vulnerability of the Co(III) metal center, but also for catalytic turnover. Indeed, the fact that the parent complex of 23-Co(III) (without an amine pendant; 4-Co(III)) showed strong DNA binding but no DNase activity suggests that the amine pendant in 23-Co(III) facilitates the catalytic activity of the complex.

Overall, these results indicate that fluoroquinolone-chelating linker conjugates and their metal complexes can be useful in treating pathogenic bacteria.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A compound represented by Formula IIa, IIb or IIc: 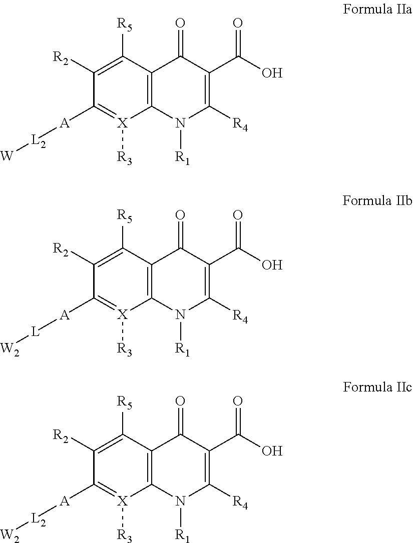
or a pharmaceutically acceptable salt thereof,
wherein:
X is C or N, wherein when X is C, the dashed line represents a bond, and when X is N, R₃ is absent;
R₁ is hydrogen, alkyl, aryl, heteroaryl or cycloalkyl, or alternatively, R₁ and R₄ or R₁ and R₃ form together a heterocyclic ring;
R₂ is hydrogen or halo;
R₃, if present, is hydrogen, alkyl, halo, alkoxy, thioalkoxy, aryloxy, thioaryloxy, aryl, heteroaryl, cyano or, alternatively, forms with R₁ a heterocyclic ring;
R₄ is hydrogen, alkyl, cycloalkyl, or halo, or, alternatively, forms with R₁ the heterocyclic ring;
R₅ is hydrogen, alkyl or cycloalkyl;
A is or comprises a heterocyclic moiety, or is or comprises a cycloalkyl substituted by an amine;
L is a linking moiety being from 6 to 10 carbon atoms in length, which can be aliphatic (non-aromatic) or aromatic;
W is a heteroalicyclic or a heteroaliphatic metal chelating moiety;
L₂ is a linking moiety being from 5 to 10 carbon atoms in length, which can be aliphatic (non-aromatic) or aromatic, and which has a moiety P that comprises a heteroatom-containing group attached thereto, wherein the heteroatom-containing group is an amine; and
W₂ is a heteroalicyclic or a heteroaliphatic metal chelating moiety which has a moiety P that comprises a heteroatom-containing group that is non-protonated or not fully protonated at physiological pH and/or is capable of reversibly binding to a metal ion when associated with the W attached thereof, wherein the heteroatom-containing group is an amine.

2. The compound of claim 1, wherein L₂ is a non-aromatic hydrocarbon chain of 5 to 10, or of 5 to 9 carbon atoms in length, wherein at least one carbon of the hydrocarbon chain is substituted by the moiety P that comprises the heteroatom-containing group.

3. The compound of claim 2, wherein the carbon atom in the hydrocarbon chain that is substituted by the moiety P is separated from the W by 0, 1 or 2 carbon atoms.

4. The compound of claim 2, wherein L₂ is 6 or 7 carbon atoms in length.

5. The compound of claim 1, wherein L₂ is —(CRaRb)-Aryl-(CRcRd)-, or —(CRaRb)-Aryl-(CRcRd)-(CReRf)—, wherein Ra-Rd, and Re and Rf, if present, are each independently hydrogen, alkyl or the moiety P that comprises the heteroatom-containing group, and wherein the Aryl is substituted by the moiety P and/or at least one of the Ra-Rd, and Re and Rf, if present, is the moiety P.

6. The compound of claim 1, wherein W₂ is a heteroalicyclic metal chelating moiety that is substituted by the moiety P that comprises the heteroatom-containing group.

7. The compound of claim 6, wherein the moiety P is attached to a heteroatom in the heteroalicyclic moiety which is ortho to the attachment point of W₂ to the L or L₂.

8. The compound of claim 1, wherein the heteroatom-containing group is a primary amine.

9. A compound represented by Formula I: 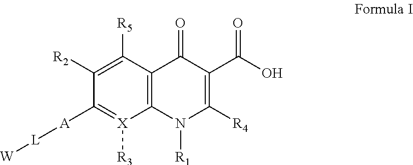
or a pharmaceutically acceptable salt thereof,
wherein:
X is C or N, wherein when X is C, the dashed line represents a bond, and when X is N, R₃ is absent;
R₁ is hydrogen, alkyl, aryl, heteroaryl or cycloalkyl, or alternatively, R₁ and R₄ or R₁ and R₃ form together a heterocyclic ring;
R₂ is hydrogen or halo;
R₃, if present, is hydrogen, alkyl, halo, alkoxy, thioalkoxy, aryloxy, thioaryloxy, aryl, heteroaryl, cyano (nitrile) or, alternatively, forms with R₁ a heterocyclic (heteroaryl or heteroalicyclic) ring;
R₄ is hydrogen, alkyl, cycloalkyl, or halo, or, alternatively, forms with R₁ the heterocyclic ring;
R₅ is hydrogen, alkyl or cycloalkyl;
A is or comprises a heterocyclic moiety, or is or comprises a cycloalkyl substituted by an amine;
L is a linking moiety (a linker) being from 6 to 10 carbon atoms in length, which can be aliphatic or aromatic; and
W is a heteroalicyclic or a heteroaliphatic metal chelating moiety.

10. The compound of claim 9, wherein L is an alkylene chain of 6 or 7 carbon atoms in length.

11. The compound of claim 10, wherein at least one carbon of the alkylene chain is substituted by a moiety P that comprises a heteroatom-containing group that is non-protonated or not fully protonated at physiological pH and/or is capable of reversibly binding to the metal ion.

12. The compound of claim 11, wherein the carbon atom in the alkylene chain that is substituted by the moiety P that comprises the heteroatom-containing moiety is separated from the W by 0, 1 or 2 carbon atoms.

13. The compound of claim 9, wherein L is —(CRaRb)-Aryl-(CRcRd)-, or —(CRaRb)-Aryl-(CRcRd)-(CReRf)—, wherein Ra-Rd, and Re and Rf, if present, are each independently hydrogen, alkyl or a moiety P that comprises a heteroatom-containing group that is non-protonated or not fully protonated at physiological pH and/or is capable of reversibly binding to the metal ion (e.g., in physiological environment).

14. The compound of claim 13, wherein the aryl is substituted by the moiety P that comprises the heteroatom-containing group and/or at least one of Rc-Rd, and Re and Rf, if present, is the moiety P that comprises the heteroatom-containing moiety.

15. The compound of claim 9, wherein the W metal chelating moiety is substituted by a moiety P that comprises a heteroatom-containing group that is non-protonated or not fully protonated at physiological pH and/or is capable of reversibly binding to the metal ion (e.g., in physiological environment).

16. The compound of claim 15, wherein the W is a heteroalicyclic moiety, and wherein the moiety P that comprises the heteroatom-containing group is attached to a heteroatom in the heteroalicyclic chelating moiety which is ortho to the attachment point to the L.

17. The compound of claim 9, comprising at least one moiety P that comprises a heteroatom-containing group that is non-protonated or not fully protonated at physiological pH and/or is capable of reversibly binding to the metal ion (e.g., in physiological environment).

18. The compound of claim 17, being represented by Formula Ia or Formula Ib: 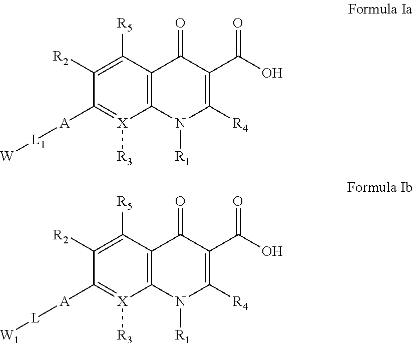
or a pharmaceutically acceptable salt thereof,
wherein:
R₁, R₂, R₃, R₄, R₅, A, L and W are as defined for Formula I;
L₁ is a non-aromatic hydrocarbon chain of from 5 to 10, or from 5 to 9, carbon atoms in length, wherein at least one carbon of the hydrocarbon chain is substituted by the moiety P that comprises the heteroatom-containing group; or
L₁ is an aromatic linker that comprises at least one aryl, wherein the aryl is substituted by the moiety P that comprises the heteroatom-containing group; and
W₁ is a heteroalicyclic metal chelating moiety that is substituted by the moiety P that comprises the heteroatom-containing group.

19. The compound of claim 18, wherein the heteroatom-containing group comprises at least one amine group which is non-protonated or not fully protonated at physiological pH.

20. The compound of claim 19, wherein the heteroatom-containing group is a guanidine group or a primary amine group.

21. The compound of claim 1, wherein W is a heteroalicyclic metal chelating moiety.

22. The compound of claim 9, wherein W is a heteroalicyclic metal chelating moiety.

23. A metal complex comprising the compound of claim 1 and a metal ion associated with the W metal chelating moiety.

24. The complex of claim 23, wherein the metal is selected from copper, cobalt, zinc, magnesium, iron, nickel and manganese.

25. A metal complex comprising the compound of claim 9 and a metal ion associated with the W metal chelating moiety, wherein the metal ion is Co(III).

26. A pharmaceutical composition comprising a compound according to claim 1, and optionally a pharmaceutically acceptable carrier.

27. A pharmaceutical composition comprising a compound according to claim 9, and optionally a pharmaceutically acceptable carrier.

28. A pharmaceutical composition comprising a metal complex according to claim 23, and optionally a pharmaceutically acceptable carrier.

29. A pharmaceutical composition comprising a metal complex according to claim 25, and optionally a pharmaceutically acceptable carrier.

30. A method of treating a medical condition associated with a pathogenic microorganism in a subject in need thereof, the method comprising administering to the subject the compound of claim 1 or a metal complex comprising a metal ion associated with the W metal chelating moiety in said compound.

31. A method of treating a medical condition associated with a pathogenic microorganism in a subject in need thereof, the method comprising administering to the subject the compound of claim 9 or a metal complex comprising a metal ion associated with the W metal chelating moiety in said compound.

32. The method of claim 31, wherein the metal is selected from Co(III) and Zn(II).
